SEQUENCE LISTING (155 sequences)

SEQ ID NO: 1 - protein, 11 aa, Artificial Sequence
  Description of Artificial Sequence Synthetic peptide
SLLMWITQVNY

SEQ ID NO: 2 - protein, 9 aa, Artificial Sequence
  Description of Artificial Sequence Synthetic peptide
EADPTGHSY

SEQ ID NO: 3 - DNA, 100 bp, Artificial Sequence
  Description of Artificial Sequence Synthetic polynucleotide
atggagtagtcagaacacactcttcagaagggactcctgatttcaaaggggggtaccgggccccccctcgagaagttcctattccgaagttcctattctc

SEQ ID NO: 4 - DNA, 100 bp, Artificial Sequence
  Description of Artificial Sequence Synthetic polynucleotide
tccgaagttcctattctctagaaagtataggaacttcctagggcgatcgcgtgcatggcactgacataggccattgttaacagggtcccagcagctggtc

SEQ ID NO: 5 - DNA, 124 bp, Artificial Sequence
  Description of Artificial Sequence Synthetic polynucleotide
gataaattattttgtcagacaacaataaaaatcaatagcacgccctaagagcggccgccaccgcggtggagctcaggtttccggtacttaacaacagagcacagatttagtggtgagggactct

SEQ ID NO: 6 - DNA, 19 bp, Artificial Sequence
  Description of Artificial Sequence Synthetic primer
ggtggagaggctattcggc

SEQ ID NO: 7 - DNA, 23 bp, Artificial Sequence
  Description of Artificial Sequence Synthetic probe
tgggcacaacagacaatcggctg

SEQ ID NO: 8 - DNA, 17 bp, Artificial Sequence
  Description of Artificial Sequence Synthetic primer
gaacacggcggcatcag

SEQ ID NO: 9 - DNA, 17 bp, Artificial Sequence
  Description of Artificial Sequence Synthetic primer
tgcggccgatcttagcc

SEQ ID NO: 10 - DNA, 21 bp, Artificial Sequence
  Description of Artificial Sequence Synthetic probe
acgagcgggttcggcccattc

SEQ ID NO: 11 - DNA, 18 bp, Artificial Sequence
  Description of Artificial Sequence Synthetic primer
ttgaccgattccttgcgg

SEQ ID NO: 12 - DNA, 20 bp, Artificial Sequence
  Description of Artificial Sequence Synthetic primer
gtcaagcactgctggcacac

SEQ ID NO: 13 - DNA, 33 bp, Artificial Sequence
  Description of Artificial Sequence Synthetic probe
aacccttgtgctattgaattgctatgctgtcag

SEQ ID NO: 14 - DNA, 20 bp, Artificial Sequence
  Description of Artificial Sequence Synthetic primer
tgttgtagaccctccgccac

SEQ ID NO: 15 - DNA, 20 bp, Artificial Sequence
  Description of Artificial Sequence Synthetic primer
ccccgtcctcctcctttttc

SEQ ID NO: 16 - DNA, 32 bp, Artificial Sequence
  Description of Artificial Sequence Synthetic probe
tcatgtccattaacccatttaccttttgccca

SEQ ID NO: 17 - DNA, 19 bp, Artificial Sequence
  Description of Artificial Sequence Synthetic primer
tgcaagtgctgccagcaag

SEQ ID NO: 18 - DNA, 27 bp, Artificial Sequence
  Description of Artificial Sequence Synthetic primer
cagtaagggaagagactacaacagcat

SEQ ID NO: 19 - DNA, 27 bp, Artificial Sequence
  Description of Artificial Sequence Synthetic probe
tgcacactgctcaccactgcaagctat

SEQ ID NO: 20 - DNA, 17 bp, Artificial Sequence
  Description of Artificial Sequence Synthetic primer
tgctggtggccccatct

SEQ ID NO: 21 - DNA, 28 bp, Artificial Sequence
  Description of Artificial Sequence Synthetic primer
gaactcagctatgatagtgtcgaatgta

SEQ ID NO: 22 - DNA, 23 bp, Artificial Sequence
  Description of Artificial Sequence Synthetic probe
cagcccagcagctgtgggttctc

SEQ ID NO: 23 - DNA, 25 bp, Artificial Sequence
  Description of Artificial Sequence Synthetic primer
gctcagggagaacacagaacttaga

SEQ ID NO: 24 - DNA, 42 bp, Artificial Sequence
  Description of Artificial Sequence Synthetic oligonucleotide
tgcgatcgctgcggccgatcttagccagacgagcgggttcgg

SEQ ID NO: 25 - DNA, 17 bp, Artificial Sequence
  Description of Artificial Sequence Synthetic primer
tgcggccgatcttagcc

SEQ ID NO: 26 - DNA, 21 bp, Artificial Sequence
  Description of Artificial Sequence Synthetic probe
acgagcgggttcggcccattc

SEQ ID NO: 27 - DNA, 18 bp, Artificial Sequence
  Description of Artificial Sequence Synthetic primer
ttgaccgattccttgcgg

SEQ ID NO: 28 - DNA, 23 bp, Artificial Sequence
  Description of Artificial Sequence Synthetic primer
cgacgtctgtcgagaagtttctg

SEQ ID NO: 29 - DNA, 24 bp, Artificial Sequence
  Description of Artificial Sequence Synthetic probe
agttcgacagcgtgtccgacctga

SEQ ID NO: 30 - DNA, 19 bp, Artificial Sequence
  Description of Artificial Sequence Synthetic primer
cacgccctcctacatcgaa

SEQ ID NO: 31 - DNA, 20 bp, Artificial Sequence
  Description of Artificial Sequence Synthetic primer
tgtcgggcgtacacaaatcg

SEQ ID NO: 32 - DNA, 22 bp, Artificial Sequence
  Description of Artificial Sequence Synthetic probe
ccgtctggaccgatggctgtgt

SEQ ID NO: 33 - DNA, 20 bp, Artificial Sequence
  Description of Artificial Sequence Synthetic primer
gggcgtcggtttccactatc

SEQ ID NO: 34 - DNA, 24 bp, Artificial Sequence
  Description of Artificial Sequence Synthetic primer
caaatggaagtagcacgtctcact

SEQ ID NO: 35 - DNA, 23 bp, Artificial Sequence
  Description of Artificial Sequence Synthetic probe
ctcgtgcagatggacagcaccgc

SEQ ID NO: 36 - DNA, 15 bp, Artificial Sequence
  Description of Artificial Sequence Synthetic primer
ccgctgccccaaagg

SEQ ID NO: 37 - DNA, 20 bp, Artificial Sequence
  Description of Artificial Sequence Synthetic primer
gtcaagcactgctggcacac

SEQ ID NO: 38 - DNA, 33 bp, Artificial Sequence; identical to SEQ ID NO: 13
  Description of Artificial Sequence Synthetic probe

SEQ ID NO: 39 - DNA, 20 bp, Artificial Sequence
  Description of Artificial Sequence Synthetic primer
tgttgtagaccctccgccac

SEQ ID NO: 40 - DNA, 21 bp, Artificial Sequence
  Description of Artificial Sequence Synthetic primer
ttgcctttctcacacctgcag

SEQ ID NO: 41 - DNA, 25 bp, Artificial Sequence
  Description of Artificial Sequence Synthetic probe
cagcccatcctgtcacttcgctgga

SEQ ID NO: 42 - DNA, 21 bp, Artificial Sequence
  Description of Artificial Sequence Synthetic primer
tggcccaacagtacagctcag

SEQ ID NO: 43 - DNA, 22 bp, Artificial Sequence
  Description of Artificial Sequence Synthetic primer
tcagtcaatcacctttcccagc

SEQ ID NO: 44 - DNA, 28 bp, Artificial Sequence
  Description of Artificial Sequence Synthetic probe
tccccaggtagcctcatgaaccaatgtt

SEQ ID NO: 45 - DNA, 23 bp, Artificial Sequence
  Description of Artificial Sequence Synthetic primer
cacattactgagtccccacaggg

SEQ ID NO: 46 - DNA, 32 bp, Artificial Sequence
  Description of Artificial Sequence Synthetic probe
accattgcagtttacccacggttaggattttt

SEQ ID NO: 47 - DNA, 23 bp, Artificial Sequence
  Description of Artificial Sequence Synthetic primer
tcttgcaatgggatcatcagatg

SEQ ID NO: 48 - DNA, 19 bp, Artificial Sequence
  Description of Artificial Sequence Synthetic primer
ggtggagaggctattcggc

SEQ ID NO: 49 - DNA, 23 bp, Artificial Sequence
  Description of Artificial Sequence Synthetic probe
tgggcacaacagacaatcggctg

SEQ ID NO: 50 - DNA, 17 bp, Artificial Sequence
  Description of Artificial Sequence Synthetic primer
gaacacggcggcatcag

SEQ ID NO: 51 - DNA, 21 bp, Artificial Sequence
  Description of Artificial Sequence Synthetic primer
gttcaggccccacagactctc

SEQ ID NO: 52 - DNA, 29 bp, Artificial Sequence
  Description of Artificial Sequence Synthetic probe
tcctctctggagcaaccatgaagttccct

SEQ ID NO: 53 - DNA, 19 bp, Artificial Sequence
  Description of Artificial Sequence Synthetic primer
cctgaagccatgagggcag

SEQ ID NO: 54 - DNA, 21 bp, Artificial Sequence
  Description of Artificial Sequence Synthetic primer
agggtaggctctcctgaatcg

SEQ ID NO: 55 - DNA, 20 bp, Artificial Sequence
  Description of Artificial Sequence Synthetic probe
acaggcgccggacctctggt

SEQ ID NO: 56 - DNA, 21 bp, Artificial Sequence
  Description of Artificial Sequence Synthetic primer
ccaaagaaactgacgcctcac

SEQ ID NO: 57 - DNA, 21 bp, Artificial Sequence
  Description of Artificial Sequence Synthetic primer
gcgccacatgaatttgaccag

SEQ ID NO: 58 - DNA, 28 bp, Artificial Sequence
  Description of Artificial Sequence Synthetic probe
tgtacccaatcttccaaagaaagagctg

SEQ ID NO: 59 - DNA, 19 bp, Artificial Sequence
  Description of Artificial Sequence Synthetic primer
ggcatcctgtcctcccttc

SEQ ID NO: 60 - DNA, 27 bp, Artificial Sequence
  Description of Artificial Sequence Synthetic primer
cagtaagggaagagactacaacagcat

SEQ ID NO: 61 - DNA, 27 bp, Artificial Sequence
  Description of Artificial Sequence Synthetic probe
tgcacactgctcaccactgcaagctat

SEQ ID NO: 62 - DNA, 17 bp, Artificial Sequence
  Description of Artificial Sequence Synthetic primer
tgctggtggccccatct

SEQ ID NO: 63 - DNA, 28 bp, Artificial Sequence
  Description of Artificial Sequence Synthetic primer
gaactcagctatgatagtgtcgaatgta

SEQ ID NO: 64 - DNA, 23 bp, Artificial Sequence
  Description of Artificial Sequence Synthetic probe
cagcccagcagctgtgggttctc

SEQ ID NO: 65 - DNA, 25 bp, Artificial Sequence
  Description of Artificial Sequence Synthetic primer
gctcagggagaacacagaacttaga

SEQ ID NO: 66 - DNA, 20 bp, Artificial Sequence
  Description of Artificial Sequence Synthetic primer
ccccgtcctcctcctttttc

SEQ ID NO: 67 - DNA, 32 bp, Artificial Sequence; identical to SEQ ID NO: 16
  Description of Artificial Sequence Synthetic probe

SEQ ID NO: 68 - DNA, 19 bp, Artificial Sequence
  Description of Artificial Sequence Synthetic primer
tgcaagtgctgccagcaag

SEQ ID NO: 69 - DNA, 156 bp, Artificial Sequence
  Description of Artificial Sequence Synthetic polynucleotide
gtcttttttgttcttcacagttgagcttcatcaaagtcacatgggttaaactctatggagtagtcagaacacactcttcagaagggactcctgatttcaaagggtaccgaagttcctattccgaagttcctattctctagaaagtataggaacttc

SEQ ID NO: 70 - DNA, 132 bp, Artificial Sequence
  Description of Artificial Sequence Synthetic polynucleotide
gaagttcctattccgaagttcctattctctagaaagtataggaacttcctagggtttcaccggtggcgcgcctaacagagaggaaagtcaaattataaagaatatgagattcagaattctgattaactgtgg

SEQ ID NO: 71 - DNA, 28 bp, Artificial Sequence
  Description of Artificial Sequence Synthetic primer
cgattatgactggttaggtagaaaggtg

SEQ ID NO: 72 - DNA, 32 bp, Artificial Sequence
  Description of Artificial Sequence Synthetic probe
gccactggtttctccaaatgttttcaatccat

SEQ ID NO: 73 - DNA, 24 bp, Artificial Sequence
  Description of Artificial Sequence Synthetic primer
gggagtacttggagatccctaagc

SEQ ID NO: 74 - DNA, 136 bp, Artificial Sequence
  Description of Artificial Sequence Synthetic polynucleotide
ttgagcttcatcaaagtcacatgggttaaactctatggagtagtcagaacacactcttcagaagggactcctgatttcaaagggtaccgaagttcctattccgaagttcctattctctagaaagtataggaacttc

SEQ ID NO: 75 - DNA, 132 bp, Artificial Sequence
  Description of Artificial Sequence Synthetic polynucleotide
gaagttcctattccgaagttcctattctctagaaagtataggaacttcctagggtttcaccggtggcgcgccaggacccaggctctgacactcaggctgccaatacaattgccatgaagacagatgttgatg

SEQ ID NO: 76 - DNA, 42 bp, Artificial Sequence; identical to SEQ ID NO: 24
  Description of Artificial Sequence Synthetic oligonucleotide

SEQ ID NO: 77 - DNA, 17 bp, Artificial Sequence
  Description of Artificial Sequence Synthetic primer
tgcggccgatcttagcc

SEQ ID NO: 78 - DNA, 21 bp, Artificial Sequence
  Description of Artificial Sequence Synthetic probe
acgagcgggttcggcccattc

SEQ ID NO: 79 - DNA, 18 bp, Artificial Sequence
  Description of Artificial Sequence Synthetic primer
ttgaccgattccttgcgg

SEQ ID NO: 80 - DNA, 23 bp, Artificial Sequence
  Description of Artificial Sequence Synthetic primer
cgacgtctgtcgagaagtttctg

SEQ ID NO: 81 - DNA, 24 bp, Artificial Sequence
  Description of Artificial Sequence Synthetic probe
agttcgacagcgtgtccgacctga

SEQ ID NO: 82 - DNA, 19 bp, Artificial Sequence
  Description of Artificial Sequence Synthetic primer
cacgccctcctacatcgaa

SEQ ID NO: 83 - DNA, 20 bp, Artificial Sequence
  Description of Artificial Sequence Synthetic primer
tgtcgggcgtacacaaatcg

SEQ ID NO: 84 - DNA, 22 bp, Artificial Sequence
  Description of Artificial Sequence Synthetic probe
ccgtctggaccgatggctgtgt

SEQ ID NO: 85 - DNA, 20 bp, Artificial Sequence
  Description of Artificial Sequence Synthetic primer
gggcgtcggtttccactatc

SEQ ID NO: 86 - DNA, 20 bp, Artificial Sequence
  Description of Artificial Sequence Synthetic primer
gtcaagcactgctggcacac

SEQ ID NO: 87 - DNA, 33 bp, Artificial Sequence; identical to SEQ ID NO: 13
  Description of Artificial Sequence Synthetic probe

SEQ ID NO: 88 - DNA, 20 bp, Artificial Sequence
  Description of Artificial Sequence Synthetic primer
tgttgtagaccctccgccac

SEQ ID NO: 89 - DNA, 21 bp, Artificial Sequence
  Description of Artificial Sequence Synthetic primer
ttgcctttctcacacctgcag

SEQ ID NO: 90 - DNA, 25 bp, Artificial Sequence
  Description of Artificial Sequence Synthetic probe
cagcccatcctgtcacttcgctgga

SEQ ID NO: 91 - DNA, 21 bp, Artificial Sequence
  Description of Artificial Sequence Synthetic primer
tggcccaacagtacagctcag

SEQ ID NO: 92 - DNA, 22 bp, Artificial Sequence
  Description of Artificial Sequence Synthetic primer
tcagtcaatcacctttcccagc

SEQ ID NO: 93 - DNA, 28 bp, Artificial Sequence
  Description of Artificial Sequence Synthetic probe
tccccaggtagcctcatgaaccaatgtt

SEQ ID NO: 94 - DNA, 23 bp, Artificial Sequence
  Description of Artificial Sequence Synthetic primer
cacattactgagtccccacaggg

SEQ ID NO: 95 - DNA, 25 bp, Artificial Sequence
  Description of Artificial Sequence Synthetic primer
cattgtcaaagaagcactggaaatg

SEQ ID NO: 96 - DNA, 32 bp, Artificial Sequence; identical to SEQ ID NO: 46
  Description of Artificial Sequence Synthetic probe

SEQ ID NO: 97 - DNA, 23 bp, Artificial Sequence
  Description of Artificial Sequence Synthetic primer
tcttgcaatgggatcatcagatg

SEQ ID NO: 98 - DNA, 19 bp, Artificial Sequence
  Description of Artificial Sequence Synthetic primer
ggtggagaggctattcggc

SEQ ID NO: 99 - DNA, 23 bp, Artificial Sequence
  Description of Artificial Sequence Synthetic probe
tgggcacaacagacaatcggctg

SEQ ID NO: 100 - DNA, 17 bp, Artificial Sequence
  Description of Artificial Sequence Synthetic primer
gaacacggcggcatcag

SEQ ID NO: 101 - DNA, 21 bp, Artificial Sequence
  Description of Artificial Sequence Synthetic primer
caggtgcaaaggtgaccacag

SEQ ID NO: 102 - DNA, 21 bp, Artificial Sequence
  Description of Artificial Sequence Synthetic probe
tgggtcctgcccatccatgca

SEQ ID NO: 103 - DNA, 20 bp, Artificial Sequence
  Description of Artificial Sequence Synthetic primer
ggcagcctgagtgtcagagc

SEQ ID NO: 104 - DNA, 21 bp, Artificial Sequence
  Description of Artificial Sequence Synthetic primer
gttcaggccccacagactctc

SEQ ID NO: 105 - DNA, 29 bp, Artificial Sequence
  Description of Artificial Sequence Synthetic probe
tcctctctggagcaaccatgaagttccct

SEQ ID NO: 106 - DNA, 19 bp, Artificial Sequence
  Description of Artificial Sequence Synthetic primer
cctgaagccatgagggcag

SEQ ID NO: 107 - DNA, 21 bp, Artificial Sequence
  Description of Artificial Sequence Synthetic primer
gcgccacatgaatttgaccag

SEQ ID NO: 108 - DNA, 28 bp, Artificial Sequence
  Description of Artificial Sequence Synthetic probe
tgtacccaatcttccaaagaaagagctg

SEQ ID NO: 109 - DNA, 19 bp, Artificial Sequence
  Description of Artificial Sequence Synthetic primer
ggcatcctgtcctcccttc

SEQ ID NO: 110 - DNA, 27 bp, Artificial Sequence
  Description of Artificial Sequence Synthetic primer
cagtaagggaagagactacaacagcat

SEQ ID NO: 111 - DNA, 27 bp, Artificial Sequence
  Description of Artificial Sequence Synthetic probe
tgcacactgctcaccactgcaagctat

SEQ ID NO: 112 - DNA, 17 bp, Artificial Sequence
  Description of Artificial Sequence Synthetic primer
tgctggtggccccatct

SEQ ID NO: 113 - DNA, 28 bp, Artificial Sequence
  Description of Artificial Sequence Synthetic primer
gaactcagctatgatagtgtcgaatgta

SEQ ID NO: 114 - DNA, 23 bp, Artificial Sequence
  Description of Artificial Sequence Synthetic probe
cagcccagcagctgtgggttctc

SEQ ID NO: 115 - DNA, 25 bp, Artificial Sequence
  Description of Artificial Sequence Synthetic primer
gctcagggagaacacagaacttaga

SEQ ID NO: 116 - DNA, 20 bp, Artificial Sequence
  Description of Artificial Sequence Synthetic primer
ccccgtcctcctcctttttc

SEQ ID NO: 117 - DNA, 32 bp, Artificial Sequence; identical to SEQ ID NO: 16
  Description of Artificial Sequence Synthetic probe

SEQ ID NO: 118 - DNA, 19 bp, Artificial Sequence
  Description of Artificial Sequence Synthetic primer
tgcaagtgctgccagcaag

SEQ ID NO: 119 - DNA, 21 bp, Artificial Sequence
  Description of Artificial Sequence Synthetic primer
tggctccaagaacagtttgcc

SEQ ID NO: 120 - DNA, 28 bp, Artificial Sequence
  Description of Artificial Sequence Synthetic probe
ccctgactttgctgctcaactcacagcc

SEQ ID NO: 121 - DNA, 21 bp, Artificial Sequence
  Description of Artificial Sequence Synthetic primer
ggtccagtggaatctgccatg

SEQ ID NO: 122 - DNA, 23 bp, Artificial Sequence
  Description of Artificial Sequence Synthetic primer
catttggctacatatcaaagccg

SEQ ID NO: 123 - DNA, 27 bp, Artificial Sequence
  Description of Artificial Sequence Synthetic probe
cctgagccagggaacagcccactgata

SEQ ID NO: 124 - DNA, 20 bp, Artificial Sequence
  Description of Artificial Sequence Synthetic primer
acatggctgaggcagacacc

SEQ ID NO: 125 - DNA, 21 bp, Artificial Sequence
  Description of Artificial Sequence Synthetic primer
tgggccgttatgctagtacca

SEQ ID NO: 126 - DNA, 25 bp, Artificial Sequence
  Description of Artificial Sequence Synthetic probe
tggctttaccccttttgaagggccc

SEQ ID NO: 127 - DNA, 21 bp, Artificial Sequence
  Description of Artificial Sequence Synthetic primer
cacagctgaagcaggatgagc

SEQ ID NO: 128 - DNA, 19 bp, Artificial Sequence
  Description of Artificial Sequence Synthetic primer
tctctgagcagccatcccc

SEQ ID NO: 129 - DNA, 27 bp, Artificial Sequence
  Description of Artificial Sequence Synthetic probe
ttctcctttggtgtagagggcaccagc

SEQ ID NO: 130 - DNA, 19 bp, Artificial Sequence
  Description of Artificial Sequence Synthetic primer
accaggcatggcagaaagg

SEQ ID NO: 131 - DNA, 156 bp, Artificial Sequence; identical to SEQ ID NO: 69
  Description of Artificial Sequence Synthetic polynucleotide

SEQ ID NO: 132 - DNA, 130 bp, Artificial Sequence
  Description of Artificial Sequence Synthetic polynucleotide
gaagttcctattccgaagttcctattctctagaaagtataggaacttcctagggtttcaccggtggcgcgcctgagtagtgctttaggtgtgtaatcaccaaagatttagtgaagtccctgtgcaaggag

SEQ ID NO: 133 - DNA, 119 bp, Artificial Sequence
  Description of Artificial Sequence Synthetic polynucleotide
ataataattaataataataaatagtaaatttctgtagaatcataatgaggtctagacccccgggctcgataactataacggtcctaaggtagcggtaccgaagttcctattccgaagtt

SEQ ID NO: 134 - DNA, 100 bp, Artificial Sequence
  Description of Artificial Sequence Synthetic polynucleotide
tctagaaagtataggaacttcctagggtttcaccggtggcgcgccgagctttctggttcagccagggacacagaaccaggaagacatcgtggcttttcta

SEQ ID NO: 135 - DNA, 106 bp, Artificial Sequence
  Description of Artificial Sequence Synthetic polynucleotide
ctttggaaaatgggactcaggttgggtgcgtctgatggagtaactgagcctctagactgagcattgcagactaatcttggatatttgtccctgagggagccggctg

SEQ ID NO: 136 - DNA, 152 bp, Artificial Sequence
  Description of Artificial Sequence Synthetic polynucleotide
aaacttcttaaaattactctattattcttccctctgattattggtctccactcgagtgccatttcattacctctttctccgcacccgacatagataaagcttggagacagctctcaacttcaccctttctgggggagcgggatgaaaaggga

SEQ ID NO: 137 - DNA, 100 bp, Artificial Sequence
  Description of Artificial Sequence Synthetic polynucleotide
gggggggtggggtggaggaggagggtacagcatctcctctccttcctctctggtaccgaagttcctattccgaagttcctattctctagaaagtatagga

SEQ ID NO: 138 - DNA, 100 bp, Artificial Sequence
  Description of Artificial Sequence Synthetic polynucleotide
tattctctagaaagtataggaacttcctagggtttcaccggtgcgatcgcatatccatgtgtgtccattctggttcagccagggacacagaaccaggaag

SEQ ID NO: 139 - DNA, 108 bp, Artificial Sequence
  Description of Artificial Sequence Synthetic polynucleotide
caggcttgcagtcctgggcagactccgtcacctctctatgcctcagccttggcgcgcctttcaaattgttgttgagttcaaagtgggcaacagaaaagggggtgtgag

SEQ ID NO: 140 - DNA, 200 bp, Artificial Sequence
  Description of Artificial Sequence Synthetic polynucleotide
aataattaataataataaatagtaaatttctgtagaatcataatgaggtctagacccccgggctcgataactataacggtcctaaggtagcgaaccggtataacttcgtataaggtatcctatacgaagttatctcgagggggggcccggtaccgattcaatgtccacacccggggctggagcgtagccatgagccacgc

SEQ ID NO: 141 - DNA, 106 bp, Artificial Sequence; identical to SEQ ID NO: 135
  Description of Artificial Sequence Synthetic polynucleotide

SEQ ID NO: 142 - DNA, 152 bp, Artificial Sequence; identical to SEQ ID NO: 136
  Description of Artificial Sequence Synthetic polynucleotide

SEQ ID NO: 143 - DNA, 23 bp, Artificial Sequence
  Description of Artificial Sequence Synthetic primer
tcaaagtcggtgaacaggcagag

SEQ ID NO: 144 - DNA, 30 bp, Artificial Sequence
  Description of Artificial Sequence Synthetic oligonucleotide
cccatgtactctgcgttgataccactgctt

SEQ ID NO: 145 - DNA, 57 bp, Artificial Sequence
  Description of Artificial Sequence Synthetic primer
gtgactggagttcagacgtgtgctcttccgatctaagcagtggtatcaacgcagagt

SEQ ID NO: 146 - DNA, 57 bp, Artificial Sequence
  Description of Artificial Sequence Synthetic primer
acactctttccctacacgacgctcttccgatctacagcaggttctgggttctggatg

SEQ ID NO: 147 - DNA, 67 bp, Artificial Sequence
  Description of Artificial Sequence Synthetic primer
aatgatacggcgaccaccgagatctacacnnnnnnacactctttccctacacgacgctcttccgatc

SEQ ID NO: 148 - DNA, 63 bp, Artificial Sequence
  Description of Artificial Sequence Synthetic primer
caagcagaagacggcatacgagatnnnnngtgactggagttcagacgtgtgctcttccgatct

SEQ ID NO: 149 - DNA, 25 bp, Artificial Sequence
  Description of Artificial Sequence Synthetic primer
cgagggtagccttttgtttgtttgc

SEQ ID NO: 150 - DNA, 30 bp, Artificial Sequence; identical to SEQ ID NO: 144
  Description of Artificial Sequence Synthetic oligonucleotide

SEQ ID NO: 151 - DNA, 58 bp, Artificial Sequence
  Description of Artificial Sequence Synthetic primer
acactctttccctacacgacgctcttccgatctgaccttgggtggagtcacatttctc

SEQ ID NO: 152 - DNA, 57 bp, Artificial Sequence; identical to SEQ ID NO: 145
  Description of Artificial Sequence Synthetic primer

SEQ ID NO: 153 - DNA, 68 bp, Artificial Sequence
  Description of Artificial Sequence Synthetic primer
aatgatacggcgaccaccgagatctacacnnnnnnacactctttccctacacgacgctcttccgatct

SEQ ID NO: 154 - DNA, 64 bp, Artificial Sequence
  Description of Artificial Sequence Synthetic primer
caagcagaagacggcatacgagatnnnnnngtgactggagttcagacgtgtgctcttccgatct

SEQ ID NO: 155 - protein, 4 aa, Artificial Sequence
  Description of Artificial Sequence Synthetic peptide
SGSG

(19) United States
(12) Patent Application Publication
(10) Pub. No.: US 2016/0081314 A1
(43) Pub. Date: Mar. 24, 2016

(54) Chimeric Antigen Receptors

(71) Applicant: Regeneron Pharmaceuticals, Inc., Tarrytown, NY (US)

(72) Inventors: Gavin Thurston, Briarcliff Manor, NY (US); Andrew J. Murphy, Croton-On-Hudson, NY (US); Lynn Macdonald, White Plains, NY (US); John Mcwhirter, Tarrytown, NY (US); Naxin Tu, Pleasantville, NY (US); Cagan Gurer, Valhalla, NY (US); Vera Voronina, Sleepy Hollow, NY (US)

(21) Appl. No.: 14/858,597

(22) Filed: Sep. 18, 2015

Related U.S. Application Data
(60) Provisional application No. 62/167,650, filed on May 28, 2015.
(60) Provisional application No. 62/094,603, filed on Dec. 19, 2014.
(60) Provisional application No. 62/076,836, filed on Nov. 7, 2014.
(60) Provisional application No. 62/052,947, filed on Sep. 19, 2014.
(60) Provisional application No. 62/052,901, filed on Sep. 19, 2014.

(51) Int. Cl. A01K 67/027; C07K 14/705

(52) U.S. Cl. CPC A01K 67/0278; C07K 14/705; A01K 2267/01; A01K 2227/105; A01K 2217/052; C07K 2319/30

(57) ABSTRACT

Provided herein are methods and compositions related to chimeric antigen receptors (CARs) having antigen binding domains derived from an immunoglobulin (Ig) and constant domains derived from a T cell receptor (TCR).

### RELATED APPLICATIONS

This application claims the benefit of priority to Provisional Application Nos. 62/167,650, filed May 28, 2015, 62/094,603, filed Dec. 19, 2014, 62/076,836, filed Nov. 7, 2014, 62/052,947, filed Sep. 19, 2014 and 62/052,901, filed Sep. 19, 2014, each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Sep. 16, 2015, is named RPB-010.01_SL.txt and is 38,779 bytes in size.

### BACKGROUND

Genetically modified rodents have proven to be a valuable source of therapeutic antibodies specific for many antigens, however some antigens have proven difficult to target. Thus, there is a great need for novel therapeutic antigen binding proteins, as well as for methods and compositions useful in the generation of such antigen binding proteins.

### SUMMARY

Provided herein are methods and compositions related to chimeric antigen receptors (CARs). For example, provided herein are CARs and CAR polypeptides, non-human animals expressing CARs and CAR polypeptides and nucleic acids encoding CARs and CAR polypeptides, as well as compositions and methods useful for the making and use of such CARs, CAR-expressing non-human animals and CAR-encoding nucleic acids.

As described herein, CARs are antigen binding proteins that have an antigen binding domain derived from an immunoglobulin (Ig) variable domain and a constant domain derived from a T cell receptor (TCR). Generally, the CARs provided herein comprise two CAR polypeptide chains, one of which includes a TCRα constant domain and one of which includes a TCRβ constant domain. Each polypeptide chain comprises an Ig variable domain, with one polypeptide chain comprising a heavy chain Ig variable domain and the other chain comprising an Ig light chain (κ or λ) variable domain. In some embodiments, the CARs provided herein have binding specificity for a peptide presented by a major histocompatibility complex (MHC) protein (e.g., a class I MHC protein or a class II MHC protein).

In certain aspects, provided herein is a genetically modified non-human animal (e.g., a rodent, such as a mouse or a rat) that expresses a CAR polypeptide. In some embodiments, the non-human animal comprises in its germline a CAR locus comprising an unrearranged variable region locus comprising unrearranged human Ig variable region gene segments (e.g., unrearranged V, D and J heavy chain gene segments, unrearranged V κ and J κ light chain gene segments or unrearranged V λ and J λ light chain gene segments) and a TCR constant region gene (e.g., a TCRα constant region gene or a TCRβ constant region gene). In some embodiments, the unrearranged Ig variable region gene segments are operably linked to the TCR constant region gene such that the genetically modified non-human animal expresses a CAR polypeptide comprising an Ig variable domain encoded by a rearranged Ig variable region gene derived from the unrearranged variable region gene segments and a TCR constant domain encoded by the TCR constant region gene. In some embodiments, the unrearranged Ig variable region gene segments are human Ig variable region gene segments. In some embodiments, the TCR constant region gene is of endogenous species origin. In some embodiments, the TCR constant domain is human or rodent (e.g., mouse or rat). In some embodiments, the TCR constant region gene is a mouse or a rat TCR constant region. In some embodiments, the unrearranged variable region locus comprises Ig variable region intergenic sequences (e.g., heavy chain intergenic sequences, κ intergenic sequences or λ intergenic sequences). In some embodiments the Ig variable region intergenic sequences are human sequences, mouse sequences or rat sequences. In some embodiments, the unrearranged variable region locus comprises TCR variable region intergenic sequences (e.g., TCRβ intergenic sequences or TCRα intergenic sequence). In some embodiments the TCR variable region intergenic sequences are human sequences, mouse sequences or rat sequences.

In some aspects, provided herein is a genetically modified non-human animal (e.g., a rodent, such as a mouse or a rat) that expresses a CAR. In some embodiments, the non-human animal comprises in its germline a first CAR locus and a second CAR locus. In some embodiments, the first CAR locus comprises an unrearranged variable region locus comprising unrearranged human Ig V_{H}, D_{H} and J_{H} gene segments and a constant region locus comprising a TCRβ constant region gene of endogenous species origin (e.g., of rat or mouse origin), wherein the human unrearranged Ig V_{H}, D_{H} and J_{H} gene segments are operably linked to the TCRβ constant region gene such that the genetically modified non-human animal expresses a first CAR polypeptide chain comprising an Ig heavy chain variable domain encoded by a rearranged heavy chain variable region gene derived from the unrearranged Ig V_{H}, D_{H} and J_{H} gene segments and a TCRβ constant domain encoded by the TCRβ constant region gene. In some embodiments, the first CAR locus comprises a rearranged variable region locus comprising a Ig heavy chain variable region gene (a universal heavy chain variable region) and a constant region locus comprising a TCRβ constant region gene of endogenous species origin (e.g., of rat or mouse origin), wherein the genetically modified non-human animal expresses a first CAR polypeptide chain comprising an Ig heavy chain variable domain encoded by the rearranged heavy chain variable region gene and a TCRβ constant domain encoded by the TCRβ constant region gene. In some embodiments, the second CAR locus comprises an unrearranged variable region locus comprising unrearranged human Ig V_{κ} and J_{κ} and a constant region locus comprising a TCRα constant region gene of endogenous species origin (e.g., of rat or mouse origin), wherein the human unrearranged Ig V_{κ} and J_{κ} gene segments are operably linked to the TCRα constant region gene such that the genetically modified non-human animal expresses a second CAR polypeptide chain comprising an Ig κ variable domain encoded by a rearranged Ig κ variable region gene derived from the unrearranged Ig V_{κ} and J_{κ} gene segments and a TCRα constant domain encoded by the TCRα constant region gene. In some embodiments, the second CAR locus comprises an unrearranged variable region locus comprising unrearranged human Ig V_{λ} and J_{λ} and a constant region locus comprising a TCRα constant region gene of endogenous species origin (e.g., of rat or mouse origin), wherein the human unrearranged Ig V_{λ} and J_{λ} gene segments are operably linked to the TCRα constant region gene such that the genetically modified non-human animal expresses a second CAR polypeptide chain comprising an Ig λ variable domain encoded by a rearranged Ig λ variable region gene derived from the unrearranged Ig V_{λ} and J_{λ} gene segments and a TCRα constant domain encoded by the TCRα constant region gene. In some embodiments, the second CAR locus comprises a rearranged variable region locus comprising a Ig light chain κ or λ variable region gene (a universal light chain variable region) and a constant region locus comprising a TCRα constant region gene of endogenous species origin (e.g., of rat or mouse origin), wherein the genetically modified non-human animal expresses a second CAR polypeptide chain comprising an Ig light chain variable domain encoded by the rearranged light chain variable region gene and a TCRα constant region encoded by the TCRα constant region gene. In some embodiments, the genetically modified non-human animal expresses a CAR comprising the first CAR polypeptide chain and the second CAR polypeptide chain. In some embodiments, one or both of the unrearranged variable region loci comprise Ig variable region intergenic sequences (e.g., heavy chain intergenic sequences, κ intergenic sequences or λ intergenic sequences). In some embodiments the Ig variable region intergenic sequences are human sequences, mouse sequences or rat sequences. In some embodiments, one or both unrearranged variable region loci comprise TCR variable region intergenic sequences (e.g., TCRβ intergenic sequences or TCRα intergenic sequence). In some embodiments the TCR variable region intergenic sequences are human sequences, mouse sequences or rat sequences.

In some embodiments, the genetically modified non-human animal described herein expresses a CAR. In some embodiments, the CAR is expressed on T cells (e.g., CD4 and/or CD8 T cells) of the non-human animal. In some embodiments, the CAR expressing T cells have undergone positive and/or negative selection in the thymus of the genetically modified non-human animal. In some embodiments, the CAR has binding specificity for a peptide/MHC complex (i.e., a peptide presented in the groove of a MHC protein). In some embodiments, the CAR has binding specificity for a peptide presented by a class I MHC protein. In some embodiments, the CAR has binding specificity for a peptide presented by a class II MHC protein.

In some embodiments of the genetically modified non-human animals described herein, the CAR locus is located at an endogenous TCR locus (e.g., an endogenous TCRα locus or an endogenous TCRβ locus). In some embodiments, the TCR constant region gene of the CAR locus is an endogenous TCR constant region gene. In some embodiments, all of or a portion of the variable region of an endogenous TCRα locus and/or TCRβ locus is replaced with all of or a portion of a variable region of an Ig locus to create the CAR locus. In some embodiments, the entire TCR variable region is replaced with an Ig variable region. In some embodiments, the TCR variable region gene segments are replaced with Ig variable region gene segments. For example, in some embodiments, the V, D and J gene segments of the endogenous TCRβ locus are replaced with Ig heavy chain V, D and J gene segments. In some embodiments, the V and J gene segments of the endogenous TCRα locus are replaced with Ig light chain (e.g., κ or λ) V and J gene segments. In some embodiments, the CAR locus is located outside of an endogenous TCR locus.

In some embodiments of the genetically modified non-human animals described herein, all of the endogenous TCR variable region gene segments in the variable region locus of the CAR locus are replaced with Ig variable region gene segments. In some embodiments substantially all TCR variable region gene segments in variable region locus of the CAR locus are replaced with Ig variable region gene segments. In some embodiments, no more than 1, 2, 3, 4, 5, 6, 7, 8, 9, or 10 TCR variable region gene segments are in the variable region locus of the CAR locus and/or are operably linked to the TCR constant region gene. In some embodiments, no functional TCR variable region gene segments are operably linked to the TCR constant region gene in the CAR locus. In some embodiments, no TCR variable region gene segments are operably linked to the TCR constant region gene in the CAR locus. In some embodiments, the CAR locus comprises at least 1, 2, 3, 4, 5, 6, 7, 8, 9, 10, 11, 12, 13, 14, 15, 16, 17, 18, 19, 20, 30, 40, 50, 60, 70 or 80 Ig variable region gene segments.

In some embodiments, the genetically modified non-human animal does not express a functional αβ TCR. In some embodiments, the genetically modified non-human animal does not express a functional TCRα chain and/or a functional TCRβ chain. In some embodiments, the endogenous TCRα variable region locus and/or TCRβ variable region locus is inactivated in the genetically modified non-human animal. For example, in some embodiments, the endogenous TCRα variable region locus and/or TCRβ variable region locus is inactivated by deletion of all of or a portion of the endogenous locus. In some embodiments, the TCRα variable region locus and/or TCRβ variable region locus is inactivated by disrupting of the operable linkage between the TCR variable region locus and the TCR constant region locus (e.g., by deleting non-coding regulatory elements, by inverting the TCR variable region locus or a portion thereof and/or by inserting nucleic acid sequence, such as nucleic acid sequence encoding an unrearranged Ig variable region or a portion thereof, between the variable region gene segments of the TCR variable region locus and the TCR constant region gene of the TCR constant region locus).

In some embodiments, the non-human animal does not comprise a TCRδ locus. TCRδ is located inside the TCRα locus, between TCRα V and TCRα J gene segments. Thus, in some embodiments, the non-human animal comprises at TCRα locus a variable region of Ig light chain comprising Ig light chain V and J gene segments operably linked to a TCRα constant region, and the TCRδ locus is either deleted or modified such that the non-human animal does not express a functional δ/γ TCR. In some embodiments, the TCRδ locus is preserved, and the non-human animal does express a functional δ/γ TCR.

In some embodiments of the genetically modified non-human animal provided herein, the unrearranged variable region of the CAR locus comprises one or more trypsinogen (TRY) genes (e.g., TRY genes and/or pseudogenes normally present in the TCRβ variable region locus). In some embodiments, the TRY genes are of endogenous species origin. In some embodiments, the TRY genes are mouse TRY genes. In some embodiments, the mouse TRY genes are selected from the group consisting of Try1, Try2, Try3, Try4, Try5, Try6, Try7, Try8, Try9, Try10, Try11, Try12, Try13, Try14, Try15, Try16, Try17, Try18, Try19 and Try20. In some embodiments, one or more TRY genes are located upstream of the V segments of the unrearranged variable region. In some embodiments, one or more TRY genes are located downstream of the V segments (e.g., downstream of the V segments and upstream of the D and/or J segments) of the unrearranged variable region. In some embodiments, Try1-7 are located upstream of the V segments of the unrearranged variable region and Try 8-20 are located downstream of the V segments (e.g., downstream of the V segments and upstream of the D and/or J segments) of the unrearranged variable region.

In some embodiments, the genetically modified non-human animal expresses one or more humanized MHC class I α chain polypeptides. In some embodiments, the humanized MHC class I α chain polypeptide is fully human. In some embodiments, the humanized MHC class I α chain polypeptide comprises a human extracellular domain (human α1, α2, and α3 domains) and a cytoplasmic domain of endogenous species origin. In some embodiments, the humanized class I α chain polypeptide is HLA-A, HLA-B, HLA-C, HLA-E, HLA-F, HLA-g, HLA-K or HLA-L. In some embodiments, the non-human animal expresses humanized HLA-A, HLA-B, HLA-C, HLA-E, HLA-F, HLA-g, HLA-K and/or HLA-L polypeptides. In some embodiments, the non-human animal comprises in its genome a humanized MHC class I α chain locus comprising a nucleic acid sequence encoding the humanized MHC class I α chain polypeptide. In some embodiments, the humanized MHC class I α chain locus is located at an endogenous MHC class I α chain locus. In some embodiments, one or more (e.g., all) of the endogenous MHC class I α chain loci of the non-human animal are replaced with humanized MHC class I α chain loci. In some embodiments, the genetically modified non-human animal does not express a MHC class I α chain polypeptide of entirely endogenous species origin.

In some embodiments, the genetically modified non-human animal expresses a humanized β-2-microglobulin polypeptide. In some embodiments, the humanized β-2-microglobulin polypeptide is fully human. In some embodiments, the non-human animal comprises in its genome a humanized β-2-microglobulin locus comprising a nucleic acid sequence encoding the humanized β-2-microglobulin polypeptide. In some embodiments, the humanized β-2-microglobulin locus is located at the endogenous β-2-microglobulin locus. In some embodiments, the endogenous β-2-microglobulin locus is replaced with the humanized β-2-microglobulin locus. In some embodiments, the genetically modified non-human animal does not express a β-2-microglobulin polypeptide of entirely endogenous species origin.

In some embodiments, the genetically modified non-human animal expresses one or more humanized MHC class II α chain polypeptides. In some embodiments, the humanized MHC class II α chain polypeptide is fully human. In some embodiments, the humanized MHC class II α chain polypeptide comprises a human extracellular domain and a cytoplasmic domain of endogenous species origin. In some embodiments, the humanized class II α chain polypeptide is HLA-DMA, HLA-DOA, HLA-DPA, HLA-DQA or HLA-DRA. In some embodiments, the non-human animal expresses humanized HLA-DMA, HLA-DOA, HLA-DPA, HLA-DQA and/or HLA-DRA polypeptides. In some embodiments, the non-human animal comprises in its genome a humanized MHC class II α chain locus comprising a nucleic acid sequence encoding the humanized MHC class II α chain polypeptide. In some embodiments, the humanized MHC class II α chain locus is located at an endogenous MHC class II α chain locus. In some embodiments, one or more (e.g., all) of the endogenous MHC class II α chain loci of the non-human animal are replaced with humanized MHC class II α chain loci. In some embodiments, the genetically modified non-human animal does not express a MHC class II α chain polypeptide of entirely endogenous species origin.

In some embodiments, the genetically modified non-human animal expresses one or more humanized MHC class II β chain polypeptides. In some embodiments, the humanized MHC class II β chain polypeptide is fully human. In some embodiments, the humanized MHC class II β chain polypeptide comprises a human extracellular domain and a cytoplasmic domain of endogenous species origin. In some embodiments, the humanized class II β chain polypeptide is HLA-DMB, HLA-DOB, HLA-DPB, HLA-DQB or HLA-DRB. In some embodiments, the non-human animal expresses humanized HLA-DMB, HLA-DOB, HLA-DPB, HLA-DQB and/or HLA-DRB polypeptides. In some embodiments, the non-human animal comprises in its genome a humanized MHC class II β chain locus comprising a nucleic acid sequence encoding the humanized MHC class II β chain polypeptide. In some embodiments, the humanized MHC class II β chain locus is located at an endogenous MHC class II β chain locus. In some embodiments, one or more (e.g., all) of the endogenous MHC class II β chain loci of the non-human animal are replaced with humanized MHC class II β chain loci. In some embodiments, the genetically modified non-human animal does not express a MHC class II β chain polypeptide of entirely endogenous species origin.

In some embodiments, the genetically modified non-human animal expresses a humanized CD8 α chain polypeptide. In some embodiments, the humanized CD8 α chain polypeptide is fully human. In some embodiments, the humanized CD8 α chain polypeptide comprises a human extracellular immunoglobulin domain and a cytoplasmic domain of endogenous species origin. In some embodiments, the non-human animal comprises in its genome a humanized CD8 α chain locus comprising a nucleic acid sequence encoding the humanized CD8 α chain polypeptide. In some embodiments, the humanized CD8 α chain locus is located at an endogenous CD8 α chain locus. In some embodiments, the endogenous CD8 α chain locus of the non-human animal is replaced with the humanized CD8 α chain locus. In some embodiments, the genetically modified non-human animal does not express a CD8 α chain polypeptide of entirely endogenous species origin.

In some embodiments, the genetically modified non-human animal expresses a humanized CD8 β chain polypeptide. In some embodiments, the humanized CD8 β chain polypeptide is fully human. In some embodiments, the humanized CD8 β chain polypeptide comprises a human extracellular immunoglobulin domain and a cytoplasmic domain of endogenous species origin. In some embodiments, the non-human animal comprises in its genome a humanized CD8 β chain locus comprising a nucleic acid sequence encoding the humanized CD8 β chain polypeptide. In some embodiments, the humanized CD8 β chain locus is located at an endogenous CD8 β chain locus. In some embodiments, the endogenous CD8 β chain locus of the non-human animal is replaced with the humanized CD8 β chain locus. In some embodiments, the genetically modified non-human animal does not express a CD8 β chain polypeptide of entirely endogenous species origin.

In some embodiments, the genetically modified non-human animal expresses a humanized CD4 polypeptide. In some embodiments, the humanized CD4 polypeptide is fully human. In some embodiments, the humanized CD4 polypeptide comprises at least one human extracellular immunoglobulin domain and a cytoplasmic domain of endogenous species origin. In some embodiments, the humanized CD4 polypeptide comprises at least a human D1 immunoglobulin domain, a human D2 immunoglobulin domain, and a human D3 immunoglobulin domain, and a cytoplasmic domain of endogenous species origin. In some embodiments, the humanized CD4 polypeptide comprises a human D1 immunoglobulin domain, a human D2 immunoglobulin domain, a human D3 immunoglobulin domain, a D4 immunoglobulin domain of endogenous species origin, and a cytoplasmic domain of endogenous species origin. In some embodiments, the non-human animal comprises in its genome a humanized CD4 locus comprising a nucleic acid sequence encoding the humanized CD4 polypeptide. In some embodiments, the humanized CD4 locus is located at an endogenous CD4 locus. In some embodiments, the endogenous CD4 locus of the non-human animal is replaced with the humanized CD4 locus. In some embodiments, the genetically modified non-human animal does not express a CD4 polypeptide of entirely endogenous species origin.

In certain aspects, provided herein is a method of making T cell expressing a CAR. In some embodiments, the CAR has antigen specificity for a peptide/MHC complex (e.g., a peptide/class I MHC complex and/or a peptide/class II MHC complex). In some embodiments, the method includes the step of exposing a genetically modified non-human animal described herein to an antigen comprising a peptide such that the peptide is presented on a MHC in the non-human animal. In some embodiments, the method includes the step of obtaining a T cell expressing a CAR specific for the peptide peptide/MHC complex from the genetically modified non-human animal. In some embodiments, provided herein is a T cell made according to and/or obtainable from a method described herein.

In certain aspects, provided herein is a method of making T cell hybridoma expressing a CAR. In some embodiments, the CAR has antigen specificity for a peptide/MHC complex (e.g., a peptide/class I MHC complex and/or a peptide/class II MHC complex). In some embodiments, the method includes the step of exposing a genetically modified non-human animal described herein to an antigen comprising a peptide such that the peptide is presented on a MHC in the non-human animal. In some embodiments, the method includes the step of obtaining a T cell expressing a CAR specific for the peptide/MHC complex from the genetically modified non-human animal. In some embodiments, the method includes the step of making a T cell hybridoma from the T cell. In some embodiments, provided herein is a T cell hybridoma made according to and/or obtainable from a method provided herein.

In certain aspects, provided herein is a method for making a nucleic acid encoding an Ig variable domain (e.g., an Ig heavy chain variable domain, an Ig κ variable domain and/or an Ig λ variable domain). In some embodiments the Ig variable domain has binding specificity for a peptide/MHC complex (e.g., a peptide/class I MHC complex and/or a peptide/class II MHC complex), either alone or when paired with another Ig variable domain. In some embodiments, the method includes the step of exposing a non-human animal described herein to an antigen comprising a peptide such that the peptide is presented on a MHC in the non-human animal. In some embodiments, the method includes the step of obtaining a T cell expressing a CAR specific for the peptide/MHC complex from the genetically modified non-human animal. In some embodiments, the method includes isolating a nucleic acid encoding an Ig variable domain of the CAR from the T cell. In some embodiments, nucleic acids encoding each of the variable domains of the CAR are isolated from the T cell. In some embodiments, provided herein is a nucleic acid encoding an Ig variable domain made according to and/or obtainable from a method provided herein.

In certain aspects, provided herein is a method for making an antibody or an antibody fragment. In some embodiments, the antibody or antibody fragment has binding specificity to a peptide/MHC complex (e.g., a peptide/class I MHC complex and/or a peptide/class II MHC complex). In some embodiments, the method includes the step of exposing a non-human animal described herein to an antigen comprising a peptide such that the peptide is presented on a MHC in the non-human animal. In some embodiments, the method includes obtaining a T cell expressing a CAR specific for the peptide/MHC complex from the genetically modified non-human animal. In some embodiments, the method includes the step of isolating nucleic acids encoding the heavy chain Ig variable domain and/or the light chain Ig variable domain of the CAR from the T cell. In some embodiments, the method includes the step of transfecting a host cell with one or more vectors encoding the heavy chain Ig variable domain and the light chain Ig variable domain such that the host cell expresses an antibody or an antibody fragment comprising the heavy chain variable domain and the light chain variable domain. In some embodiments, the method includes the step of operably linking a nucleic acid sequence encoding the heavy chain Ig variable domain with a nucleic acid sequence encoding a heavy chain Ig constant domain in a host cell such that the host cell expresses an Ig heavy chain polypeptide comprising the Ig heavy chain variable domain and the Ig heavy chain constant domain. In some embodiments, the method includes the step of operably linking a nucleic acid sequence encoding the light chain Ig variable domain with a nucleic acid sequence encoding a light chain Ig constant domain in a host cell such that the host cell expresses an Ig light chain polypeptide comprising the Ig light chain variable domain and the Ig heavy chain constant domain. In some embodiments, the method includes the step of operably linking a nucleic acid sequence encoding the heavy chain Ig variable domain with a nucleic acid sequence encoding a heavy chain Ig constant domain in a host cell and operably linking a nucleic acid sequence encoding the light chain Ig variable domain with a nucleic acid sequence encoding a light chain Ig constant domain in the host cell such that the host cell expresses an antibody having a heavy chain comprising the heavy chain Ig variable domain and the heavy chain Ig constant domain and a light chain comprising the light chain Ig variable domain and the light chain Ig constant domain. In some embodiments, the method includes the step of culturing the host cell under conditions such that the host cell expresses an antibody or antibody fragment. In some embodiments, the Ig light chain and/or heavy chain constant domain are human Ig constant domains. In some embodiments, provided herein is an antibody or antibody fragment made according to and/or obtainable from a method provided herein.

In certain embodiments, provided herein are methods of treating cancer in a subject comprising administering to the subject an antibody or antibody fragment described herein (e.g., an antibody that has binding specificity to a peptide/MHC complex and/or that has been generated according to a method described herein). In some embodiments, the methods described herein may be used to treat any cancerous or pre-cancerous tumor. Cancers that may treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments, provided herein are methods of treating a subject suffering from an infection, including a viral infection, a bacterial infection, a helminth infection, or a protozoan infection, comprising administering to the subject an antibody or antibody fragment described herein (e.g., an antibody that has binding specificity to a peptide/MHC complex and/or that has been generated according to a method described herein). For example, in some embodiments, provided herein are methods of treating viral infectious diseases, including HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. In some embodiments, the pathogen treated are parasites, such as malaria. In some embodiments, provided herein are treatments of bacterial, fungal and other pathogenic diseases, such as Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum.

In some aspects, provided herein is a method for making a cell (e.g., a human cell, such as a human T cell) expressing a CAR. In some embodiments, the antibody or antibody fragment has binding specificity to a peptide/MHC complex (e.g., a peptide/class I MHC complex and/or a peptide/class II MHC complex). In some embodiments, the method includes the step of exposing a non-human animal described herein to an antigen comprising a peptide such that the peptide is presented on a MHC in the non-human animal. In some embodiments, the method includes obtaining a T cell expressing a CAR specific for the peptide presented on the MHC from the genetically modified non-human animal. In some embodiments, the method includes the step of isolating nucleic acids encoding the heavy chain Ig variable domain and/or the light chain Ig variable domain of the CAR from the T cell. In some embodiments, the method includes the step of operably linking a nucleic acid sequence encoding the heavy chain Ig variable domain with a nucleic acid sequence encoding a TCR constant domain (e.g., a TCRβ constant domain or a TCRα constant domain) in a cell (e.g., a human cell, such as a human T cell) such that the cell expresses a CAR polypeptide comprising the Ig heavy chain variable domain and the TCR constant domain. In some embodiments, the method includes the step of operably linking a nucleic acid sequence encoding the light chain Ig variable domain with a nucleic acid sequence encoding a TCR constant domain (e.g., a TCRβ constant domain or a TCRα constant domain) in a cell (e.g., a human cell, such as a human T cell) such that the cell expresses a CAR polypeptide comprising the Ig light chain variable domain and the TCR constant domain. In some embodiments, the method includes the step of operably linking a nucleic acid sequence encoding the heavy chain Ig variable domain with a first TCR constant domain (e.g., a TCRβ constant domain or a TCRα constant domain) in a cell (e.g., a human cell, such as a human T cell) and operably linking a nucleic acid sequence encoding the light chain Ig variable domain with a nucleic acid sequence encoding a second TCR constant domain (e.g., a TCRβ constant domain if the first TCR constant domain is a TCRα constant domain or a TCRα constant domain if the first TCR constant domain is a TCRβ constant domain) in the cell such that the cell expresses an CAR having a first CAR chain polypeptide comprising the heavy chain Ig variable domain and the first TCR constant domain and a second CAR polypeptide comprising the light chain Ig variable domain and the second TCR constant domain. In some embodiments, the TCR constant domains are human TCR constant domains. In some embodiments, the cell is an ex-vivo cell (e.g., an ex vivo human cell, such as an ex vivo human T cell). In some embodiments, provided herein is a cell expressing a CAR made according to and/or obtainable from a method provided herein.

In some embodiments, any method of exposing the genetically modified non-human animal described herein to an antigen comprising a peptide such that the peptide is presented on a MHC in the non-human animal can be used. In some embodiments, the genetically modified non-human animal is exposed to the antigen by infecting the non-human animal with a virus (e.g., a retrovirus, an adenovirus or a lentivirus) comprising a nucleic acid sequence encoding the antigen. In some embodiments, the genetically modified non-human animal is exposed to the antigen by administering to the animal a nucleic acid encoding the peptide such that the peptide is expressed in the non-human animal. In some embodiments, the non-human animal is administered a nucleic acid encoding a single chain peptide/MHC complex. In some embodiments, the genetically modified non-human animal is exposed to the antigen by administering to the genetically modified non-human animal a peptide/MHC complex. In some embodiments, the non-human animal is administered a single chain peptide/MHC complex (e.g., a single chain ecto-MHC/β-2-microglobulin/peptide protein complex). In some embodiments, the peptide/MHC complex is administered as a multimer (e.g., a tetramer). In some embodiments, the peptide/MHC complex is present on the surface of a cell (e.g., an antigen presenting cell, such as a macrophage or dendritic cell). In some embodiments, a B7.1, B7.2 or ICOS-L is present on the surface of the cell. In some embodiments, the cell expresses a T cell stimulatory cytokine (e.g. IL-1, IL-2, IL-4, IL-6, IL-12, IL-13, IFN-γ, TNF-α, TGF-β, IFN-α and/or IFN-β).

In some embodiments of the methods described herein, any method can be used to isolate the nucleic acid comprising encoding the Ig variable domain. In some embodiments, the step of isolating the nucleic acid comprises making a T cell hybridoma from the T cell and isolating the nucleic acid from the T cell hybridoma. In some embodiments, the nucleic acid is isolated using a nucleic acid amplification process (e.g., PCR). In some embodiments, the nucleic acid is isolated by sequencing the rearranged Ig variable region gene in a CAR locus of the T cell or the T cell hybridoma and synthesizing a nucleic acid sequence comprising the rearranged Ig variable region gene.

In certain aspects, provided herein is a cell expressing a CAR obtained from or obtainable from a genetically modified non-human animal described herein. In some embodiments, the cell is a T cell. In some embodiments, the cell is a T cell hybridoma. In some embodiments, the CAR has binding specificity for a peptide/MHC complex.

In certain aspects, provided herein is a nucleic acid comprising a rearranged Ig variable region gene (e.g., a heavy chain Ig variable region gene or a light chain heavy chain variable region gene) obtained from or obtainable from a genetically modified non-human animal or cell described herein. In some embodiments, the nucleic acid further comprises a TCR constant region gene (e.g., a TCRα constant region gene or a TCRβ constant region gene). In some embodiments, the nucleic acid encodes a CAR polypeptide. In some embodiments, the Ig variable region gene encodes an Ig variable domain that has binding specificity for a peptide/MHC complex.

In certain aspects, provided herein is a CAR or a CAR polypeptide obtained from or obtainable from a genetically modified non-human animal or cell described herein. In some embodiments, the CAR or CAR polypeptide has binding specificity for a peptide/MHC complex.

In certain aspects, provided herein is a non-human embryonic stem (ES) cell (e.g., a rodent ES cell, such as a mouse ES cell or a rat ES cell) that comprises in its genome a CAR locus. In some embodiments, the CAR locus comprises an unrearranged variable region locus comprising unrearranged human Ig variable region gene segments (e.g., unrearranged V, D and J heavy chain gene segments, unrearranged V κ and J κ light chain gene segments or unrearranged V λ and J λ light chain gene segments) and a TCR constant region gene (e.g., a TCRα constant region gene or a TCRβ constant region gene). In some embodiments, the unrearranged Ig variable region gene segments are operably linked to the TCR constant region gene. In some embodiments, the unrearranged Ig variable region gene segments are human Ig variable region gene segments. In some embodiments, the TCR constant region gene is of endogenous species origin. In some embodiments, the unrearranged variable region locus comprises Ig variable region intergenic sequences (e.g., heavy chain intergenic sequences, κ intergenic sequences or λ intergenic sequences). In some embodiments the Ig variable region intergenic sequences are human sequences, mouse sequences or rat sequences. In some embodiments, the unrearranged variable region locus comprises TCR variable region intergenic sequences (e.g., TCRβ intergenic sequences or TCRα intergenic sequence). In some embodiments the TCR variable region intergenic sequences are human sequences, mouse sequences or rat sequences.

In some aspects, provided herein is a non-human ES cell (e.g., a rodent ES cell, such as a mouse ES cell or a rat ES cell) that comprises in its genome a first CAR locus and a second CAR locus. In some embodiments, the first CAR locus comprises an unrearranged variable region locus comprising unrearranged human Ig V_{H}, D_{H} and J_{H} gene segments and a constant region locus comprising a TCRβ constant region gene of endogenous species origin, wherein the human unrearranged Ig V_{H}, D_{H} and J_{H} gene segments are operably linked to the TCRβ constant region gene. In some embodiments, the first CAR locus comprises a rearranged variable region locus comprising a Ig heavy chain variable region gene (a universal heavy chain variable region) and a constant region locus comprising a TCRβ constant region gene of endogenous species origin. In some embodiments, the second CAR locus comprises a an unrearranged variable region locus comprising unrearranged human Ig V_{κ} and J_{κ} and a constant region locus comprising a TCRα constant region gene of endogenous species origin, wherein the human unrearranged Ig V_{κ} and J_{κ} gene segments are operably linked to the TCRα constant region gene. In some embodiments, the second CAR locus comprises an unrearranged variable region locus comprising unrearranged human Ig V_{λ} and J_{λ} and a constant region locus comprising a TCRα constant region gene of endogenous species origin, wherein the human unrearranged Ig V_{λ} and J_{λ} gene segments are operably linked to the TCRα constant region gene. In some embodiments, the second CAR locus comprises a rearranged variable region locus comprising a Ig light chain κ or λ variable region gene (a universal light chain variable region) and a constant region locus comprising a TCRα constant region gene of endogenous species origin. In some embodiments, one or both of the unrearranged variable region loci comprises Ig variable region intergenic sequences (e.g., heavy chain intergenic sequences, κ intergenic sequences or λ intergenic sequences). In some embodiments the Ig variable region intergenic sequences are human sequences, mouse sequences or rat sequences. In some embodiments, one or both of the unrearranged variable region loci comprise TCR variable region intergenic sequences (e.g., TCRβ intergenic sequences or TCRα intergenic sequence). In some embodiments the TCR variable region intergenic sequences are human sequences, mouse sequences or rat sequences.

In some embodiments of the non-human animal ES cells described herein, the CAR locus is located at an endogenous TCR locus (e.g., an endogenous TCRα locus or an endogenous TCRβ locus). In some embodiments, the TCR constant region gene of the CAR locus is an endogenous TCR constant region gene. In some embodiments, all of or a portion of the variable region of an endogenous TCRα locus and/or TCRβ locus is replaced with all of or a portion of a variable region of an Ig locus to create the CAR locus. In some embodiments, the entire TCR variable region is replaced with an Ig variable region. In some embodiments, the TCR variable region gene segments are replaced with Ig variable region gene segments. For example, in some embodiments, the V, D and J gene segments of the endogenous TCRβ locus are replaced with Ig heavy chain V, D and J gene segments. In some embodiments, the V and J gene segments of the endogenous TCRα locus are replaced with Ig light chain (e.g., κ or λ) V and J gene segments.

In some embodiments, the non-human ES cell does not comprise a functional TCR locus. In some embodiments, the non-human ES cell does not comprise a functional TCRα chain locus and/or a functional TCRβ chain locus. In some embodiments, the endogenous TCRα locus and/or TCRβ locus is inactivated in the genetically modified non-human ES cell (e.g., by deletion of all of or a portion of the endogenous locus). In some embodiments, the non-human ES cell does not comprise a functional TCRδ locus.

In some embodiments of the non-human animal ES cells described herein, the unrearranged variable region of the CAR locus comprises one or more trypsinogen (TRY) genes (e.g., TRY genes and/or pseudogenes normally present in the TCRβ variable region locus). In some embodiments, the TRY genes are of endogenous species origin. In some embodiments, the TRY genes are mouse TRY genes. In some embodiments, the mouse TRY genes are selected from the group consisting of Try1, Try2, Try3, Try4, Try5, Try6, Try7, Try8, Try9, Try10, Try11, Try12, Try13, Try14, Try15, Try16, Try17, Try18, Try19 and Try20. In some embodiments, one or more TRY genes are located upstream of the V segments of the unrearranged variable region. In some embodiments, one or more TRY genes are located downstream of the V segments (e.g., downstream of the V segments and upstream of the D and/or J segments) of the unrearranged variable region. In some embodiments, Try1-7 are located upstream of the V segments of the unrearranged variable region and Try 8-20 are located downstream of the V segments (e.g., downstream of the V segments and upstream of the D and/or J segments) of the unrearranged variable region.

In some embodiments, the non-human ES cell comprises in its genome a locus encoding a humanized MHC class I α chain polypeptides. In some embodiments, the humanized MHC class I α chain polypeptide is fully human. In some embodiments, the humanized MHC class I α chain polypeptide comprises a human extracellular domain (human α1, α2, and α3 domains) and a cytoplasmic domain of endogenous species origin. In some embodiments, the humanized class I α chain polypeptide is HLA-A, HLA-B, HLA-C, HLA-E, HLA-F, HLA-g, HLA-K or HLA-L. In some embodiments, the non-human ES cell comprises loci encoding humanized HLA-A, HLA-B, HLA-C, HLA-E, HLA-F, HLA-g, HLA-K and/or HLA-L polypeptides. In some embodiments, the humanized MHC class I α chain locus is located at an endogenous MHC class I α chain locus. In some embodiments, one or more (e.g., all) of the endogenous MHC class I α chain loci of the non-human ES cell are replaced, in whole or in part, with humanized MHC class I α chain loci. In some embodiments, the non-human ES cell does not comprise a functional endogenous MHC class I α chain locus (e.g., a locus encoding a MHC class I α chain of entirely endogenous species origin).

In some embodiments, the non-human ES cell comprises in its genome a locus encoding a humanized β-2-microglobulin polypeptide. In some embodiments, the humanized β-2-microglobulin polypeptide is fully human. In some embodiments, the humanized β-2-microglobulin locus is located at the endogenous β-2-microglobulin locus. In some embodiments, the endogenous β-2-microglobulin locus is replaced, in whole or in part, with the humanized β-2-microglobulin locus. In some embodiments, the non-human ES cell does not comprise in its genome a functional endogenous β-2-microglobulin locus (e.g., a locus encoding a β-2-microglobulin polypeptide of entirely endogenous species origin).

In some embodiments, the non-human ES cell comprises in its genome a locus encoding a humanized MHC class II α chain polypeptide. In some embodiments, the humanized MHC class II α chain polypeptide is fully human. In some embodiments, the humanized MHC class II α chain polypeptide comprises a human extracellular domain and a cytoplasmic domain of endogenous species origin. In some embodiments, the humanized class II α chain polypeptide is HLA-DMA, HLA-DOA, HLA-DPA, HLA-DQA or HLA-DRA. In some embodiments, the non-human ES cell comprises loci encoding humanized HLA-DMA, HLA-DOA, HLA-DPA, HLA-DQA and/or HLA-DRA polypeptide. In some embodiments, the humanized MHC class II α chain locus is located at an endogenous MHC class II α chain locus. In some embodiments, one or more (e.g., all) of the endogenous MHC class II α chain loci of the non-human ES cell are replaced, in whole or in part, with humanized MHC class II α chain loci. In some embodiments, the genetically modified non-human ES cell does not comprise in its genome a functional endogenous MHC class II α chain locus (e.g., a locus encoding a MHC class II α chain of entirely endogenous species origin).

In some embodiments, the non-human ES cell comprises in its genome a locus encoding a humanized MHC class II β chain polypeptide. In some embodiments, the humanized MHC class II β chain polypeptide is fully human. In some embodiments, the humanized MHC class II β chain polypeptide comprises a human extracellular domain and a cytoplasmic domain of endogenous species origin. In some embodiments, the humanized class II β chain polypeptide is HLA-DMB, HLA-DOB, HLA-DPB, HLA-DQB or HLA-DRB. In some embodiments, the non-human ES cell comprises in its genome loci encoding humanized HLA-DMB, HLA-DOB, HLA-DPB, HLA-DQB and/or HLA-DRB polypeptides. In some embodiments, the humanized MHC class II 3 chain locus is located at an endogenous MHC class II β chain locus. In some embodiments, one or more (e.g., all) of the endogenous MHC class II β chain loci of the non-human ES cell are replaced, in whole or in part, with humanized MHC class II β chain loci. In some embodiments, the non-human ES cell does not comprise in its genome a functional endogenous MHC class II β chain locus (e.g., a locus encoding a MHC class II β chain of entirely endogenous species origin).

In some embodiments, the non-human ES cell comprises in its genome a locus encoding a humanized CD8 α chain polypeptide. In some embodiments, the humanized CD8 α chain polypeptide is fully human. In some embodiments, the humanized CD8 α chain polypeptide comprises a human extracellular immunoglobulin domain and a cytoplasmic domain of endogenous species origin. In some embodiments, the humanized CD8 α chain locus is located at an endogenous CD8 α chain locus. In some embodiments, the endogenous CD8 α chain locus of the non-human ES cell is replaced, in whole or in part, with the humanized CD8 α chain locus. In some embodiments, the non-human ES cell does not comprise in its genome a functional endogenous CD8 α chain locus (e.g., a locus encoding a CD8 α chain of entirely endogenous species origin).

In some embodiments, the non-human ES cell comprises in its genome a locus encoding a humanized CD8 β chain polypeptide. In some embodiments, the humanized CD8 β chain polypeptide is fully human. In some embodiments, the humanized CD8 β chain polypeptide comprises a human extracellular immunoglobulin domain and a cytoplasmic domain of endogenous species origin. In some embodiments, the humanized CD8 β chain locus is located at an endogenous CD8 β chain locus. In some embodiments, the endogenous CD8 β chain locus of the non-human ES cell is replaced, in whole or in part, with the humanized CD8 β chain locus. In some embodiments, the non-human ES cell does not comprise in its genome a functional endogenous CD8 β chain locus (e.g., a locus encoding a CD8 β chain of entirely endogenous species origin).

In some embodiments, the non-human ES cell comprises in its genome a locus encoding a humanized CD4 polypeptide. In some embodiments, the humanized CD4 polypeptide is fully human. In some embodiments, the humanized CD4 polypeptide comprises at least a human D1 immunoglobulin domain, a human D2 immunoglobulin domain, and a human D3 immunoglobulin domain, and a cytoplasmic domain of endogenous species origin. In some embodiments, the humanized CD4 polypeptide comprises a human D1 immunoglobulin domain, a human D2 immunoglobulin domain, a human D3 immunoglobulin domain, a D4 immunoglobulin domain of endogenous species origin, and a cytoplasmic domain of endogenous species origin. In some embodiments, the humanized CD4 locus is located at an endogenous CD4 locus. In some embodiments, the endogenous CD4 locus of the non-human ES cell is replaced, in whole or in part, with the humanized CD4 locus. In some embodiments, the non-human ES cell does not comprise in its genome a functional endogenous CD4 chain locus (e.g., a locus encoding a CD4 chain of entirely endogenous species origin).

In certain aspects, provided herein is a genetically modified non-human animal generated using or obtainable from an ES cell described herein. In some embodiments, the genetically modified non-human animal is a rodent. In some embodiments, the genetically modified non-human animal is a mouse or a rat. In some embodiments, provided herein is a non-human embryo comprising a non-human ES cell described herein.

In certain aspects, provided herein is a method of making a genetically modified non-human animal that expresses a CAR and/or a CAR polypeptide. In certain embodiments, the method comprises using a non-human ES cell described herein to generate a non-human animal. In certain embodiments, the non-human ES cell is a mouse non-human ES cell. In some embodiments, the method comprises using the VELOCIMOUSE® method, as described in U.S. Pat. No. 7,294,754, which is hereby incorporated by reference. In certain embodiments, provided herein is a genetically modified non-human animal generated using or obtainable from the methods provided herein.

In certain aspects, provided herein is a CAR locus comprising an unrearranged variable region locus comprising unrearranged human Ig variable region gene segments; and a constant region locus comprising a rodent TCR constant region gene (e.g., a mouse TCR constant region gene or a rat TCR constant region gene), wherein the human unrearranged Ig variable region gene segments are operably linked to the TCR constant region gene. In some embodiments, the unrearranged Ig variable region gene segments are human Ig heavy chain (IgH) variable region gene segments. In some embodiments, the unrearranged Ig variable region gene segments are human Ig light chain (IgL) variable region gene segments (e.g., Ig κ gene segments or Ig λ gene segments). In some embodiments, the TCR constant region gene is a TCRα constant region gene. In some embodiments, the CAR locus is located at an endogenous TCRα locus. In some embodiments, the unrearranged human Ig variable region gene segments replace endogenous TCRα variable region gene segments. In some embodiments, the TCRα constant region gene is an endogenous TCRα constant region gene. In some embodiments, the TCR constant region gene is a TCRβ constant region gene. In some embodiments, the CAR locus is located at an endogenous TCRβ locus. In some embodiments, the unrearranged human Ig variable region gene segments replace endogenous TCRβ variable region gene segments. In some embodiments, the TCRβ constant region gene is an endogenous TCRβ constant region gene. In some embodiments, the unrearranged variable region locus further comprises one or more trypsinogen genes. In some embodiments, the unrearranged variable region locus comprises Ig variable region intergenic sequences (e.g., heavy chain intergenic sequences, κ intergenic sequences or λ intergenic sequences). In some embodiments the Ig variable region intergenic sequences are human sequences, mouse sequences or rat sequences. In some embodiments, the unrearranged variable region locus comprises TCR variable region intergenic sequences (e.g., TCRβ intergenic sequences or TCRα intergenic sequence). In some embodiments the TCR variable region intergenic sequences are human sequences, mouse sequences or rat sequences.

In certain aspects, provided herein is a CAR locus comprising an unrearranged variable region locus comprising unrearranged human Ig V_{H}, D_{H} and J_{H} gene segments and a constant region locus comprising a rodent TCRβ constant region gene (e.g., a rat TCRβ constant region gene or a mouse TCRβ constant region gene), wherein the human unrearranged Ig V_{H}, D_{H} and J_{H} gene segments are operably linked to the TCRβ constant region gene. In certain embodiments, the CAR locus is located at an endogenous TCRβ locus. In certain embodiments, the unrearranged human Ig V_{H}, D_{H} and J_{H} gene segments replace endogenous TCRβ variable region gene segments. In some embodiments, the TCRβ constant region gene is an endogenous TCRβ constant region gene. In some embodiments, the unrearranged variable region locus further comprises one or more trypsinogen genes. In some embodiments, the unrearranged variable region locus comprises Ig variable region intergenic sequences (e.g., heavy chain intergenic sequences, κ intergenic sequences or λ intergenic sequences). In some embodiments the Ig variable region intergenic sequences are human sequences, mouse sequences or rat sequences. In some embodiments, the unrearranged variable region locus comprises TCR variable region intergenic sequences (e.g., TCRβ intergenic sequences or TCRα intergenic sequence). In some embodiments the TCR variable region intergenic sequences are human sequences, mouse sequences or rat sequences.

In certain aspects, provided herein is a CAR locus comprising an unrearranged variable region locus comprising unrearranged human Ig V_{κ} and J_{κ} gene segments and a constant region locus comprising a rodent TCRα constant region gene, wherein the human unrearranged Ig V_{κ} and J_{κ} gene segments are operably linked to the TCRα constant region gene. In some embodiments, the CAR locus is located at an endogenous TCRα locus. In some embodiments, the unrearranged human Ig V_{κ} and J_{κ} gene segments replace endogenous TCRα variable region gene segments. In some embodiments, the TCRα constant region gene is an endogenous TCRα constant region gene. In some embodiments, the CAR locus does not comprise a functional TCRδ locus; in some embodiments, a TCRδ locus is deleted.

In certain aspects, provided herein is a rodent (e.g., a rat or a mouse) comprising in its germline a CAR locus described herein. In some aspects, provided herein is a rodent cell (e.g., a rat cell or a mouse cell) comprising in its germline a CAR locus described herein. In some embodiments, the cell is an ES cell. In some embodiments, provided herein is a nucleic acid (e.g., a vector) encoding a CAR locus described herein. In some embodiments, the unrearranged variable region locus comprises Ig variable region intergenic sequences (e.g., heavy chain intergenic sequences, κ intergenic sequences or λ intergenic sequences). In some embodiments the Ig variable region intergenic sequences are human sequences, mouse sequences or rat sequences. In some embodiments, the unrearranged variable region locus comprises TCR variable region intergenic sequences (e.g., TCRβ intergenic sequences or TCRα intergenic sequence). In some embodiments the TCR variable region intergenic sequences are human sequences, mouse sequences or rat sequences.

In certain aspects, provided herein is a method of making a non-human animal (e.g., a mouse or a rat) that expresses a CAR described herein. In certain embodiments, the method comprises genetically modifying a non-human animal such that it comprises a CAR locus described herein in its germline. In some embodiments, the method comprises genetically modifying a non-human ES cell (e.g., a mouse ES cell or a rat ES cell) such that it comprises a CAR locus described herein. In some embodiments, the method comprises introducing into a non-human ES cell a CAR locus comprising unrearranged Ig light chain gene segments (light chain V and J segments) operably linked to a TCRα constant region, and introducing into a non-human ES cell a CAR locus comprising unrearranged Ig heavy chain gene segments (heavy chain V, D, and J segments) operably linked to a TCRβ constant region. In some embodiments, the method comprises modifying a TCRα locus of a non-human animal ES cell to comprise unrearranged Ig light chain gene segments (light chain V and J segments) operably linked to a TCRα constant region, and modifying a TCRβ locus of a non-human animal ES cell to comprise unrearranged Ig heavy chain gene segments (heavy chain V, D, and J segments) operably linked to a TCRβ constant region.

In certain aspects, provided herein is a chimeric antigen receptor (CAR) comprising a first CAR polypeptide comprising an Ig heavy chain variable domain and a TCRβ constant domain and a second CAR polypeptide comprising an Ig light chain variable domain (e.g., an Ig κ variable domain or an Ig λ variable domain) and a TCRα constant domain, wherein the CAR has binding specificity for a peptide/MHC complex (see, e.g., FIG. 1). In some embodiments, the peptide/MHC complex is a peptide/class I MHC complex. In some embodiments, the peptide/MHC complex is a peptide/class II MHC complex. In some embodiments, the Ig heavy chain variable domain and/or the Ig light chain variable domain are human Ig variable domains. In some embodiments, the TCRβ constant domain and/or the TCRα constant domain are rodent constant domains (e.g., rat or mouse constant domains). In some embodiments, the TCRβ constant domain and/or the TCRα constant domain are human constant domains.

In certain aspects, provided herein is a chimeric antigen receptor (CAR) comprising a first CAR polypeptide comprising an Ig heavy chain variable domain and a TCRα constant domain and a second CAR polypeptide comprising an Ig light chain variable domain (e.g., an Ig κ variable domain or an Ig λ variable domain) and a TCRβ constant domain, wherein the CAR has binding specificity for a peptide/MHC complex (see, e.g., FIG. 2). In some embodiments, the peptide/MHC complex is a peptide/class I MHC complex. In some embodiments, the peptide/MHC complex is a peptide/class II MHC complex. In some embodiments, the Ig heavy chain variable domain and/or the Ig light chain variable domain are human Ig variable domains. In some embodiments, the TCRβ constant domain and/or the TCRα constant domain are rodent constant domains (e.g., rat or mouse constant domains). In some embodiments, the TCRβ constant domain and/or the TCRα constant domain are human constant domains.

In certain aspects, provided herein is a cell or non-human animal expressing a CAR described herein. In some embodiments, the cell is a T cell. In some embodiments, the cell or animal is a human or rodent (e.g., rat or mouse). In certain embodiments, provided herein is a non-human animal (e.g., a rodent, such as a rat or a mouse) comprising a cell described herein.

In some aspects, provided herein is a method of inducing an immune response to a peptide/MHC complex in a subject. In some embodiments, the method includes administering to the subject a cell (e.g., a human T cell, such as a CD4 T cell or a CD8 T cell) expressing a CAR comprising a first CAR polypeptide comprising a human Ig heavy chain variable domain and a human TCRβ constant domain and a second CAR polypeptide comprising a human Ig light chain variable domain (e.g., an Ig κ variable domain or an Ig λ variable domain) and a human TCRα constant domain, wherein the CAR has binding specificity for the peptide/MHC complex. In some embodiments, the peptide/MHC complex is a peptide/class I MHC complex. In some embodiments, the peptide/MHC complex is a peptide/class II MHC complex.

In some aspects, provided herein is a method of inducing an immune response to a peptide/MHC complex in a subject (e.g., a human subject). In some embodiments, the method includes administering to the subject a cell (e.g., a human T cell, such as a CD4 T cell or a CD8 T cell) expressing a CAR comprising a first CAR polypeptide comprising a human Ig heavy chain variable domain and a human TCRα constant domain and a second CAR polypeptide comprising a human Ig light chain variable domain (e.g., an Ig κ variable domain or an Ig λ variable domain) and a human TCRβ constant domain, wherein the CAR has binding specificity for the peptide/MHC complex. In some embodiments, the peptide/MHC complex is a peptide/class I MHC complex. In some embodiments, the peptide/MHC complex is a peptide/class II MHC complex.

In certain aspects, provided herein is a method of inducing an immune response to a peptide/MHC complex in a subject (e.g., a human subject). In some embodiments, the method includes isolating a T cell (e.g., a CD4 T cell or a CD8 T cell) from the subject. In some embodiments, the method includes inducing expression by the T cell of a CAR comprising a first CAR polypeptide comprising a human Ig heavy chain variable domain and a human TCRβ constant domain and a second CAR polypeptide comprising a human Ig light chain variable domain (e.g., an Ig κ variable domain or an Ig λ variable domain) and a human TCRα constant domain, wherein the CAR has binding specificity for the peptide/MHC complex. In some embodiments, the method includes administering the T cell to the subject. In some embodiments, the method comprises transfecting the T cell with a first vector comprising a nucleic acid sequence encoding the first CAR polypeptide and a second vector comprising a nucleic acid sequence encoding the second CAR polypeptide. In some embodiments, the method comprises transfecting the T cell with a vector comprising a nucleic sequence encoding the first CAR polypeptide and a nucleic acid sequence encoding the second CAR polypeptide. In some embodiments, the method comprises the step of inhibiting expression by the T cell of endogenous TCRα and/or TCRβ.

In certain aspects, provided herein is a method of inducing an immune response to a peptide/MHC complex in a subject (e.g., a human subject). In some embodiments, the method includes isolating a T cell (e.g., a CD4 T cell or a CD8 T cell) from the subject. In some embodiments, the method includes inducing expression by the T cell of a CAR comprising a first CAR polypeptide comprising a human Ig heavy chain variable domain and a human TCRα constant domain and a second CAR polypeptide comprising a human Ig light chain variable domain (e.g., an Ig κ variable domain or an Ig λ variable domain) and a human TCRβ constant domain, wherein the CAR has binding specificity for the peptide/MHC complex. In some embodiments, the method includes administering the T cell to the subject. In some embodiments, the method comprises transfecting the T cell with a first vector comprising a nucleic acid sequence encoding the first CAR polypeptide and a second vector comprising a nucleic acid sequence encoding the second CAR polypeptide. In some embodiments, the method comprises transfecting the T cell with a vector comprising a nucleic sequence encoding the first CAR polypeptide and a nucleic acid sequence encoding the second CAR polypeptide. In some embodiments, the method comprises the step of inhibiting expression by the T cell of endogenous TCRα and/or TCRβ.

In certain aspects, provided herein is a nucleic acid composition comprising a first nucleic acid sequence encoding a first CAR polypeptide comprising an Ig heavy chain variable domain and a TCRβ constant domain and a second nucleic acid sequence encoding a second CAR polypeptide comprising an Ig light chain variable domain (e.g., an Ig κ variable domain or an Ig λ variable domain) and a TCRα constant domain, wherein a CAR comprising the first CAR polypeptide and the second CAR polypeptide has binding specificity for a peptide/MHC complex. In certain embodiments, the Ig heavy chain variable domain and/or the Ig light chain variable domain are human Ig variable domains. In some embodiments, the TCRβ constant domain and/or the TCRα constant domain are rodent constant domains (e.g., rat constant domains or mouse constant domains). In some embodiments, the TCRβ constant domain and/or the TCRα constant domain are human constant domains. In some embodiments, the first nucleic acid sequence and the second nucleic acid sequence are on a single nucleic acid molecule. In some embodiments, the first nucleic acid sequence and the second nucleic acid sequence are on separate nucleic acid molecules.

In certain aspects, provided herein is a nucleic acid composition comprising a first nucleic acid sequence encoding a first CAR polypeptide comprising an Ig heavy chain variable domain and a TCRα constant domain and a second nucleic acid sequence encoding a second CAR polypeptide comprising an Ig light chain variable domain (e.g., an Ig κ variable domain or an Ig λ variable domain) and a TCRβ constant domain, wherein a CAR comprising the first CAR polypeptide and the second CAR polypeptide has binding specificity for a peptide/MHC complex. In certain embodiments, the Ig heavy chain variable domain and/or the Ig light chain variable domain are human Ig variable domains. In some embodiments, the TCRβ constant domain and/or the TCRα constant domain are rodent constant domains (e.g., rat constant domains or mouse constant domains). In some embodiments, the TCRβ constant domain and/or the TCRα constant domain are human constant domains. In some embodiments, the first nucleic acid sequence and the second nucleic acid sequence are on a single nucleic acid molecule. In some embodiments, the first nucleic acid sequence and the second nucleic acid sequence are on separate nucleic acid molecules.

In certain aspects, provided herein is a method of making a cell that expresses a CAR comprising transfecting the cell with a nucleic acid composition described herein. In some embodiments, the cell is a human cell. In some embodiments, the cell is a rodent cell (e.g., a rat cell or a mouse cell). In some embodiments, the cell is a T cell. In some embodiments, the cell is an ex vivo T cell. In some embodiments, provided herein is a cell made according to or obtainable from a method described herein.

In certain aspects, provided herein is a method of treating a disease or disorder in a subject comprising administering to the subject a T cell expressing a CAR described herein. In some embodiments, the disease or disorder is cancer and the CAR has binding specificity for a MHC presented cancer antigen. In some embodiments, the disease or disorder is an infectious disease and the CAR has binding specificity for a pathogen antigen (e.g., a viral, bacterial or parasitic antigen). In some embodiments, the disease or disorder is an autoimmune and/or inflammatory disorder and the CAR is specific for an autoimmune self-antigen and expressed by a regulatory T cell. In some embodiments, the T cell is a CD4⁺ T cell. In some embodiments, the T cell is a CD8⁺ T cell.

### BRIEF DESCRIPTION OF THE DRAWINGS

FIG. 1 depicts a schematic representation of a an exemplary CAR described herein interacting with a peptide/MHC complex on an antigen presenting cell.

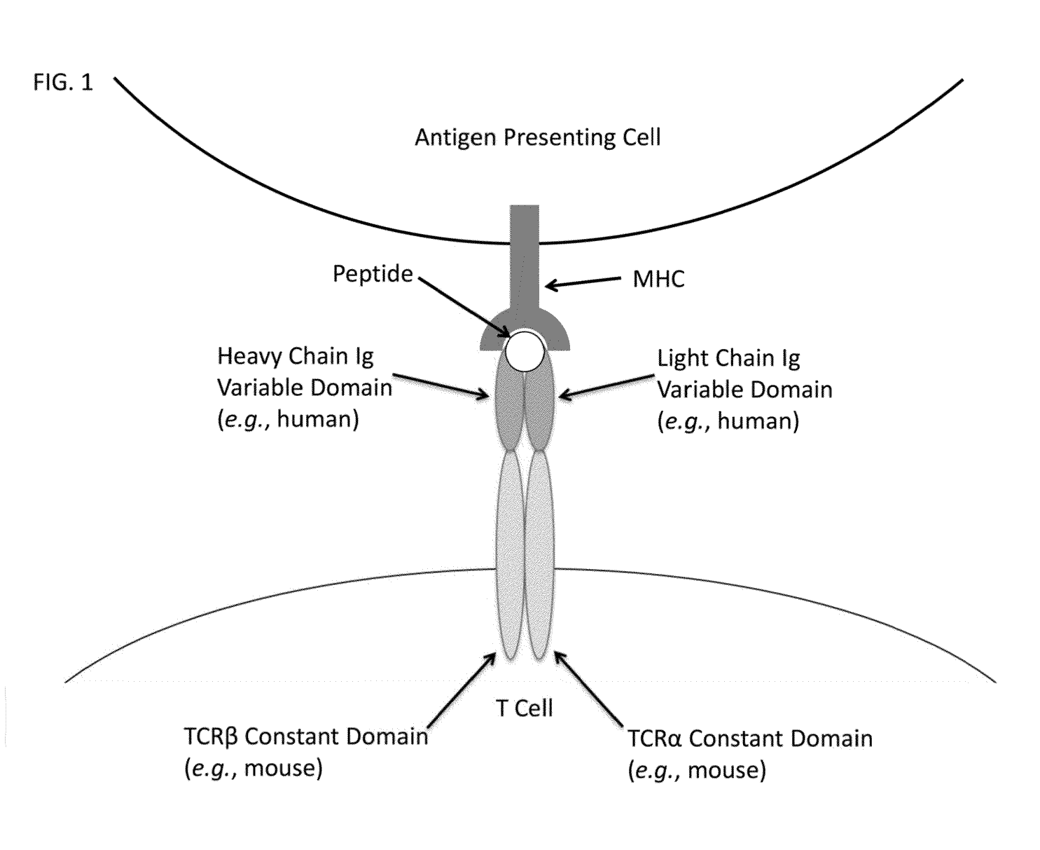

FIG. 2 depicts a schematic representation of a an exemplary CAR described herein interacting with a peptide/MHC complex on an antigen presenting cell.

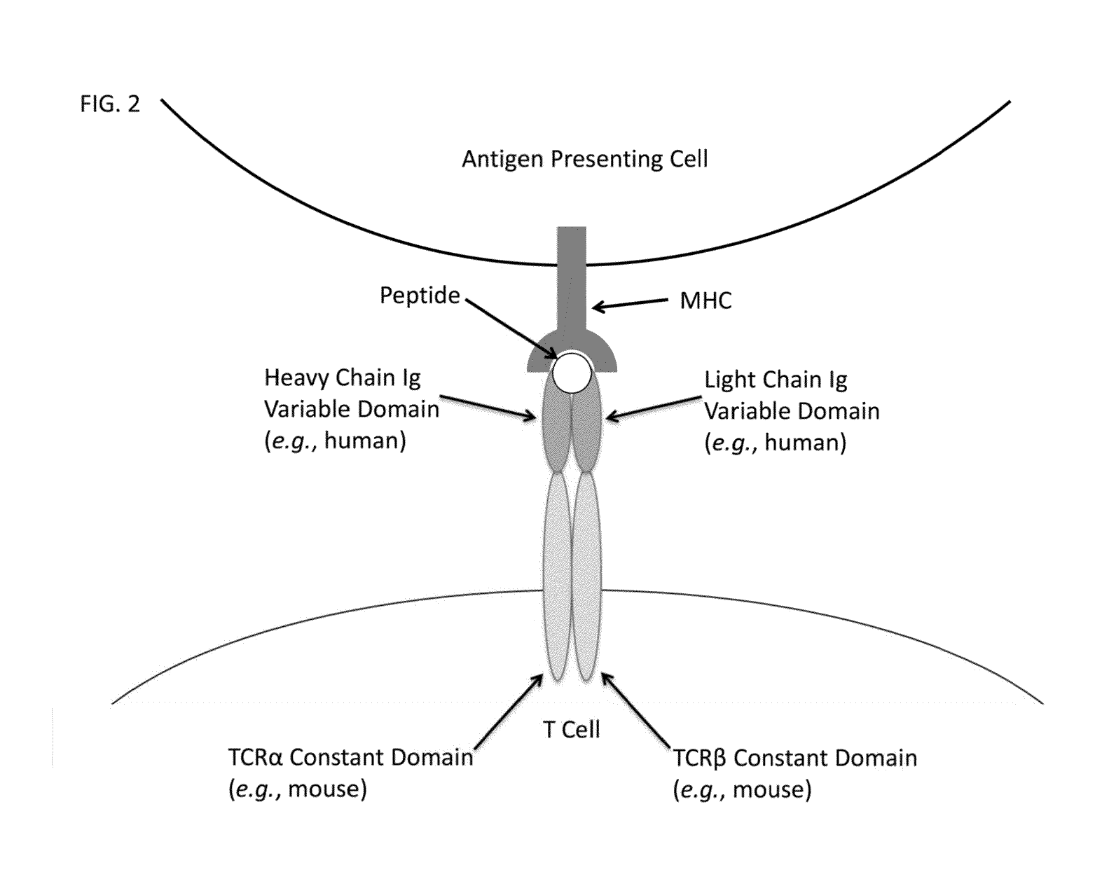

FIG. 3 depicts an exemplary scheme for linking human Ig variable regions to a mouse TCR locus using lentiviral vectors. Figure discloses “SGSG” as SEQ ID NO: 155.

FIG. 4 shows cytokine secretion by CAR expressing cells in response to antigen presentation.

FIG. 5 depicts an exemplary scheme (not to scale) for the creation of a large targeting vector (LTVEC) for inserting Ig κ variable region gene segments to the mouse endogenous TCRα locus. Unless specifically indicated otherwise (e.g., selection cassettes, etc.), mouse sequences are depicted by filled shapes and single lines, and human sequences are depicted by empty shapes and double lines. Certain restriction sites used for cloning are indicated.

FIG. 6 depicts an exemplary scheme (not to scale) for inserting Ig κ variable region gene segments to the mouse endogenous TCRα locus. Unless specifically indicated otherwise, mouse sequences are depicted by filled shapes and single lines, and human sequences are depicted by empty shapes and double lines. TAQMAN probe hybridization locations are indicated.

FIG. 7 depicts an exemplary scheme (not to scale) for inserting additional Igκ variable region gene segments to the mouse endogenous TCRα locus. Unless specifically indicated otherwise, mouse sequences are depicted by filled shapes and single lines, and human sequences are depicted by empty shapes and double lines. TAQMAN probe hybridization locations are indicated.

FIG. 8 depicts an exemplary scheme (not to scale) for inserting additional Igκ variable region gene segments to the mouse endogenous TCRα locus. Unless specifically indicated otherwise, mouse sequences are depicted by filled shapes and single lines, and human sequences are depicted by empty shapes and double lines. TAQMAN probe hybridization locations are indicated.

FIG. 9 depicts an exemplary scheme (not to scale) for inserting additional Igκ variable region gene segments to the mouse endogenous TCRα locus. Unless specifically indicated otherwise, mouse sequences are depicted by filled shapes and single lines, and human sequences are depicted by empty shapes and double lines. TAQMAN probe hybridization locations are indicated.

FIG. 10 depicts an exemplary scheme (not to scale) for the creation of a LTVEC for inserting Ig heavy chain variable region gene segments to the mouse endogenous TCRβ locus. Unless specifically indicated otherwise, mouse sequences are depicted by filled shapes and single lines, and human sequences are depicted by empty shapes and double lines.

FIG. 11 depicts an exemplary scheme (not to scale) for inserting Ig heavy chain variable region gene segments to the mouse endogenous TCRβ locus. Unless specifically indicated otherwise, mouse sequences are depicted by filled shapes and single lines, and human sequences are depicted by empty shapes and double lines.

FIG. 12 depicts exemplary LTVECs (not to scale) useful for inserting Ig heavy chain variable region gene segments to the mouse endogenous TCRβ locus. Unless specifically indicated otherwise, mouse sequences are depicted by filled shapes and single lines, and human sequences are depicted by empty shapes and double lines.

FIG. 13 depicts an exemplary scheme (not to scale) for scheme for inserting Ig heavy chain variable region gene segments to the mouse endogenous TCRβ locus. Step 4 depicts an optional step of deleting TCR Vβ31 gene segment. Unless specifically indicated otherwise, mouse sequences are depicted by filled shapes and single lines, and human sequences are depicted by empty shapes and double lines.

FIG. 14 depicts the V_{κ} and J_{κ} usage during rearrangement of an Ig κ/TCRα CAR locus in thymocytes and splenic T cells of a transgenic mouse in which an endogenous TCRα variable region locus has been replaced with an unrearranged Ig κ variable region gene segments (4 functional V_{κ} and 5 functional J_{κ}). IGKV7-3 is a pseudogene.

FIG. 15 depicts the productive versus non-productive rearrangements of an Ig κ/TCRα CAR locus in splenic T cells of three transgenic mice in which an endogenous TCRα variable region locus has been replaced with an unrearranged Ig κ variable region gene segments (4 functional V_{κ} and 5 functional J_{κ}). Productive rearrangements (“prod”) here include rearrangements where the rearranged nucleic acid sequence can be translated into a protein having, in operable linkage, the sequence of V_{κ}, followed by the sequence of J_{κ}, followed by the sequence of TCRα constant domain. Nonproductive rearrangements (“nonprod”) include those where the rearranged VκJκ exons are out of frame with a nucleic acid sequence encoding TCRα constant domain, or are in frame with a sequence encoding a TCRα domain but containing a stop codon so that they cannot be translated into protein.

FIG. 16 depicts the V_{κ} and J_{κ} usage during rearrangement of an Ig κ/TCRα CAR locus in thymocytes and splenic T cells of a transgenic mouse in which an endogenous TCRα variable region locus has been replaced with an unrearranged Ig κ variable region gene segments (16 functional V_{κ} and 5 functional J_{κ}).

FIG. 17 depicts the productive versus non-productive rearrangements of an Ig κ/TCRα CAR locus in thymocytes and splenic T cells of transgenic mice in which an endogenous TCRα variable region locus has been replaced with an unrearranged Ig κ variable region gene segments (16 functional V_{κ} and 5 functional J_{κ}). Productive rearrangements (“prod”) here include rearrangements where the rearranged nucleic acid sequence can be translated into a protein having, in operable linkage, the sequence of V_{κ}, followed by the sequence of J_{κ}, followed by the sequence of TCRα constant domain. Nonproductive rearrangements (“nonprod”) include those where the rearranged VκJκ exons are out of frame with a sequence encoding a TCRα constant domain, or are in frame with TCRα but containing a stop codon so that they cannot be translated into protein.

FIG. 18 depicts the V_{H} and J_{H} usage during rearrangement of an IgH/TCRβ CAR locus in thymocytes and splenic T cells of a transgenic mouse in which an endogenous TCRβ variable region locus has been replaced with an unrearranged IgH variable region gene segments (3 functional V_{H} and all functional D and J_{H}).

FIG. 19 depicts the productive versus non-productive rearrangements of an IgH/TCRβ CAR locus in thymocytes and splenic T cells of transgenic mice in which an endogenous TCRβ variable region locus has been replaced with an unrearranged IgH variable region gene segments (3 functional V_{H} and all functional D and J_{H}). Productive rearrangements (“prod”) here include rearrangements where the rearranged nucleic acid sequence can be translated into a protein having, in operable linkage, the sequence of V_{H}, followed by the sequence of D, followed by the sequence of J_{H}, followed by the sequence of TCRβ constant domain. Nonproductive rearrangements (“nonprod”) include those where the rearranged VDJ exons are out of frame with a sequence encoding a TCRβ constant domain, or are in frame with TCRβ but containing a stop codon so that they cannot be translated into protein.

### DETAILED DESCRIPTION

### General

Provided herein are methods and compositions related to chimeric antigen receptors (CARs) having antigen binding domains derived from an immunoglobulin (Ig) and constant domains derived from a T cell receptor (TCR). In some embodiments, the CAR has binding specificity for a peptide presented by a major histocompatibility complex (MHC) protein.

Antibodies have proven to be valuable therapeutic agents due to their ability to bind to target antigens with high affinity and specificity. One of the weaknesses of existing antibody therapeutic technologies is the difficulty of targeting certain antigens, such as intracellular antigens, due to the challenges associated with delivering antibodies across a cellular membrane. Thus, current antibody therapeutics are generally directed to extracellular antigens, such as cell surface proteins and soluble factors, such as cytokines. On the other hand, intracellular targets, including many tumor antigens and viral antigens, remain difficult to target.

The challenge of delivering antibodies across a cellular membrane could be avoided through the use of antibodies that were able to recognize peptide antigens presented on major histocompatibility complex (MHC) proteins. All nucleated mammalian cells process endogenous cellular proteins into peptides that are loaded onto class I MHC proteins and presented on the surface of the cell. Similarly, professional antigen presenting cells (APCs), such as dendritic cells or macrophages, process exogenous antigens into peptides that are loaded onto class II MHC proteins and presented on the APC cell surface. During T cell development in the thymus, T cells undergo positive and negative selection, which ensures that only the small minority of T cells expressing TCR with very weak peptide-independent affinity to MHC emerge from the thymus (positive selection) while T cells expressing TCR with moderate to high affinity to self-peptide/MHC are driven to apoptosis (negative selection). Antibodies, unlike TCR, do not normally undergo MHC-based positive and negative selection, and it has proven difficult to generate antibodies specific for peptide/MHC complexes using conventional antibody generation techniques.

As described herein, in some embodiments soluble antigen binding molecules, such as antibodies, specific for peptide/MHC complexes can be generated using genetically modified non-human animals (e.g., mice) that are engineered to have T cells that express CARs having Ig variable domains and TCR constant domains. Such non-human animals have Ig variable domains derived from unrearranged Ig light and heavy chain variable (V(D)J) gene segments operably linked to TCRα and TCRβ constant regions and, upon encountering an antigen (e.g., peptide/MHC) undergo V(D)J rearrangement at the CAR loci to generate rearranged CAR molecules that result in CAR expression on T cells. Because such T cells undergo positive and negative selection, the CARs expressed have antigen specificity for peptide/MHC. Such mice can therefore be used to generate antigen binding proteins able to target peptide/MHC complexes. For example, the mice can be immunized with a peptide/MHC antigen such that antigen specific T cells are generated. The nucleic acid encoding the Ig variable domains of the CARs expressed on the antigen specific T cells can be operably linked to nucleic acid encoding Ig constant domains in a host cell such that the host cell expresses a peptide/MHC specific antibody.

### DEFINITIONS

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable domain and a heavy chain constant domain. Each light chain includes a light chain variable domain and a light chain constant domain. The heavy chain variable domains and light chain variable domains can be further subdivided into domains of hypervariability, termed complementarity determining regions (CDR), interspersed with regions that are more conserved, termed framework regions (FR). Each heavy chain variable domain and light chain variable domain is composed of three CDRs and four FRs, arranged from amino-terminus to carboxy-terminus in the following order: FR1, CDR1, FR2, CDR2, FR3, CDR3, FR4. The variable domains of the heavy and light chains contain a binding domain that interacts with an antigen.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, single-chain antibodies, isolated CDRH3, and other antibody fragments that retain at least a portion of the variable domain of an intact antibody. These antibody fragments can be obtained using conventional recombinant and/or enzymatic techniques and can be screened for antigen binding in the same manner as intact antibodies.

As used herein, a “chimeric antigen receptor” or “CAR” refers to an antigen binding protein in that includes an immunoglobulin antigen binding domain (e.g., an immunoglobulin variable domain) and a T cell receptor (TCR) constant domain. As used herein, a “constant domain” of a TCR polypeptide includes a membrane-proximal TCR constant domain, and may also include a TCR transmembrane domain and/or a TCR cytoplasmic tail. For example, in some embodiments, the CAR is a dimer that includes a first polypeptide comprising a immunoglobulin heavy chain variable domain linked to a TCRβ constant domain and a second polypeptide comprising an immunoglobulin light chain variable domain (e.g., a κ or λ variable domain) linked to a TCRα constant domain. In some embodiments, the CAR is a dimer that includes a first polypeptide comprising a immunoglobulin heavy chain variable domain linked to a TCRα constant domain and a second polypeptide comprising an immunoglobulin light chain variable domain (e.g., a κ or λ variable domain) linked to a TCRβ constant domain.

The phrase “derived from” when used concerning a rearranged variable region gene “derived from” an unrearranged variable region and/or unrearranged variable region gene segments refers to the ability to trace the sequence of the rearranged variable region gene back to a set of unrearranged variable region gene segments that were rearranged to form a gene that expresses the variable domain (accounting for, where applicable, splice differences and somatic mutations). For example, a rearranged variable region gene that has undergone somatic mutation is still derived from the unrearranged variable region gene segments. In some embodiments, where the endogenous locus is replaced with a universal light chain or heavy chain locus, the term “derived from” indicates the ability to trace origin of the sequence to said rearranged locus even though the sequence may have undergone somatic mutations.

As used herein, the term “locus” refers to a location on a chromosome that contains a set of related genetic elements (e.g., genes, gene segments, regulatory elements). For example, an unrearranged immunoglobulin locus may include immunoglobulin variable region gene segments, one or more immunoglobulin constant region genes and associated regulatory elements (e.g., promoters, enhancers, switch elements, etc.) that direct V(D)J recombination and immunoglobulin expression. Similarly, an unrearranged CAR locus may include immunoglobulin variable region gene segments, a TCR constant region gene and associated regulatory elements (e.g., promoters, enhancers, etc.) that direct V(D)J recombination and CAR expression. A locus can be endogenous or non-endogenous. The term “endogenous locus” refers to a location on a chromosome at which a particular genetic element is naturally found. For example, an endogenous mouse TCRα locus refers to the location on mouse chromosome 14 that includes TCRα variable region gene segments and constant region genes in a wild-type mouse, while an endogenous mouse TCRβ locus refers to the location on mouse chromosome 6 that includes TCRβ variable region gene segments and constant region genes in a wild-type mouse.

Unrearranged variable region gene segments are “operably linked” to a contiguous constant region gene if the unrearranged variable region gene segments are capable of rearranging to form a rearranged variable region gene that is expressed in conjunction with the constant region gene as a polypeptide chain of an antigen binding protein. For example, unrearranged immunoglobulin variable region gene segments are operably linked to a TCR constant region gene in a CAR locus.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, “specific binding” and “antigen specificity” refers to the ability of an antigen binding molecule (e.g., an antibody or CAR) to bind to a predetermined target, such as a predetermined peptide/MHC complex. Typically, an antigen binding molecule specifically binds to its predetermined target with an affinity corresponding to a K_{D} of about 10⁻⁷ M or less, and binds to the predetermined target with an affinity (as expressed by K_{D}) that is at least 10 fold less, at least 100 fold less or at least 1000 fold less than its affinity for binding to a non-specific and unrelated target (e.g., BSA, casein).

The term “unrearranged” includes the state of an immunoglobulin, TCR or CAR variable region locus or variable region gene segments wherein V gene segments and J gene segments (for heavy or TCRβ variable regions, D gene segments as well) are maintained separately but are capable of being joined to form a rearranged V(D)J gene (a “variable region gene”) that comprises a single V, (D), J of the V(D)J repertoire.

### Chimeric Antigen Receptor Loci

In certain aspects, provided herein are chimeric antigen receptor (CAR) loci. Such CAR loci generally comprise a variable region locus and a constant region locus. The variable region locus includes unrearranged Ig variable region gene segments, while the constant region locus includes a TCR constant region gene, wherein the Ig variable region gene segments are operably linked to the constant region gene. In some embodiments the variable region will be an unrearranged variable region and will therefore contain unrearranged Ig variable region gene segments. In some embodiments, the variable region will be a rearranged variable region and will therefore contain a rearranged variable region gene. In certain embodiments, the Ig variable region gene segments are human variable region gene segments and the TCR constant region gene is a non-human constant region gene. For example, in some embodiments, the TCR constant region gene is a rodent constant region gene, such as a rat constant region gene or a mouse constant region gene. In certain embodiments, the Ig variable region gene segments are human variable region gene segments and the TCR constant region gene is a human constant region gene.

In some embodiments, the CAR loci described herein are located at an endogenous TCR loci. For example, in some embodiments, a CAR locus comprising a TCRα constant region gene is located at an endogenous TCRα constant region locus. In some embodiments, such a locus is created by replacing some or all of the TCRα unrearranged variable region with an unrearranged Ig variable region. In some embodiments, a CAR locus comprising a TCRβ constant region gene is located at an endogenous TCRβ constant region locus. In some embodiments, such a locus is created by replacing some or all of the TCRβ unrearranged variable region with an unrearranged Ig variable region. Methods for the construction of exemplary CAR loci are provided herein in Example 2.

In certain embodiments, the CAR variable region locus will contain unrearranged human Ig variable region gene segments. Exemplary variable region loci comprising human variable region gene segments have been described in the art. For example, such loci are described in U.S. Pat. Nos. 5,770,429, 5,814,318, 6,114,598, 6,998,514, 8,232,449, 8,502,018 and 8,697,940, each of which is hereby incorporated by reference, and in U.S. Pat. Pub. Nos. 2008/0098490, 2012/0167237, 2013/0145484, 2013/0326647, 2014/013275 and 2014/093908, each of which is hereby incorporated by reference.

In certain embodiments, the CAR variable region locus contains unrearranged human Ig heavy chain variable region gene segments. In some embodiments, the unrearranged human Ig variable region gene segments comprise a plurality of human V_{H} segments, one or more human D_{H} segments and one or more human J_{H} segments. In some embodiments, the unrearranged human Ig variable region gene segments comprise at least 3 V_{H} gene segments, at least 18 V_{H} gene segments, at least 20 V_{H} gene segments, at least 30 V_{H} gene segments, at least 40 V_{H} gene segments, at least 50 V_{H} gene segments, at least 60 V_{H} gene segments, at least 70 V_{H} gene segments, or at least 80 V_{H} gene segments. In some embodiments, the unrearranged human Ig gene segments include all of the human D_{H} gene segments. In some embodiments, the CAR variable region further comprises TCRβ variable region gene segments (e.g., V, D and/or J gene segments). In one embodiment, the CAR variable region further comprises distal TCR Vβ gene segments, e.g., TCR Vβ31 gene segment. In another embodiment, the distal TCR Vβ gene segments, e.g., TCR Vβ31 gene segment, has been functionally inactivated or deleted. In some embodiments, the unrearranged human Ig gene segments include all of the human J_{H} gene segments. Exemplary variable regions comprising Ig heavy chain gene segments are provided, for example, in Macdonald et al., Proc. Nati. Acad. Sci. USA 111:5147-52 and supplemental information, which is hereby incorporated by reference.

In some embodiments, the CAR variable gene locus comprising unrearranged human Ig heavy chain variable region gene segments also includes human Ig heavy chain variable region intergenic sequences. In some embodiments, the CAR variable gene locus includes non-human (e.g., rodent, rat, mouse) Ig heavy chain variable region intergenic sequences. In some embodiments, the CAR variable gene locus includes human or non-human (e.g., rodent, rat, mouse) TCRβ variable region intergenic sequences. For example, in some embodiments the unrearranged variable region of the CAR locus comprises one or more (e.g., 1, 2, 3, 4, 5, 6, 7, 8, 9, 10, 11, 12, 13, 14, 15, 16, 17, 18, 19 or 20) trypsinogen (TRY) genes (e.g., TRY genes and/or pseudogenes normally present in the TCRβ variable region locus). In some embodiments, the TRY genes are mouse TRY genes. In some embodiments, the mouse TRY genes are selected from the group consisting of Try1, Try2, Try3, Try4, Try5, Try6, Try7, Try8, Try9, Try10, Try11, Try12, Try13, Try14, Try15, Try16, Try17, Try18, Try19 and Try20. In some embodiments, one or more TRY genes are located upstream of the V_{H} segments of the unrearranged variable region. In some embodiments, one or more TRY genes are located downstream of the V_{H} segments and upstream of the D_{H} segments of the unrearranged variable region. In some embodiments, Try1-7 are located upstream of the V_{H} segments of the unrearranged variable region and Try 8-20 are located downstream of the V_{H} segments and upstream of the D_{H} segments of the unrearranged variable region. Additional information regarding the TRY genes located in the human and/or mouse TCRβ locus is provided in Glusman et al., Immunity 15:337-349 (2001) and Skok et al., Nature Immunology 8:378-387 (2007), each of which is incorporated by reference. In some embodiments, the CAR gene locus comprises non-human regulatory elements (e.g., non-human promoters and/or enhancers. In some embodiments, the non-human regulatory elements are rodent regulatory elements (e.g., rat or mouse promoters or enhancers). In some embodiments, the CAR locus comprises an IgM enhancer (Eμ). In some embodiments, the IgM enhancer is a non-human Eμ (e.g., a rodent Eμ, such as a mouse or rat Eμ).

In certain embodiments, the CAR variable region locus contains unrearranged human Ig κ variable region gene segments. In some embodiments, the unrearranged human immunoglobulin variable region gene segments comprise a plurality of human V_{κ} segments and one or more human J_{κ} segments. In some embodiments, the immunoglobulin variable region gene segments comprise four functional V_{κ} segments and all human J_{κ} segments. In some embodiments, the immunoglobulin variable region gene segments comprise 16 functional V_{κ} segments and all human J_{κ} segments. In some embodiments, the unrearranged human immunoglobulin variable region gene segments comprise all of the human Vκ segments and all human J_{κ} segments. Exemplary variable regions comprising Ig κ gene segments are provided, for example, in Macdonald et al., Proc. Natl. Acad. Sci. USA 111:5147-52 and supplemental information, which is hereby incorporated by reference. In some embodiments, the unrearranged human immunoglobulin variable region gene segments comprise all of the human Jκ segments. In some embodiments, the CAR variable region further comprises TCRα variable region gene segments (e.g., V, and/or J gene segments).

In certain embodiments, the CAR variable region locus contains unrearranged human Ig λ variable region gene segments. In some embodiments, the unrearranged human immunoglobulin variable region gene segments comprise a plurality of human V_{λ} segments and one or more human J_{λ} segments. In some embodiments, the unrearranged human immunoglobulin variable region gene segments comprise all of the human V_{λ} segments. In some embodiments, the unrearranged human immunoglobulin variable region gene segments comprise all of the human J_{λ} segments. In some embodiments, the CAR variable region further comprises TCRα variable region gene segments (e.g., V, and/or J gene segments). Exemplary variable regions comprising Ig λ gene segments are provided, for example, U.S. Pat. Pub. Nos. 2012/0073004 and 2002/0088016, each of which is hereby incorporated by reference.

In some embodiments, the CAR variable gene locus containing unrearranged human Ig light chain variable region gene segments also includes human Ig light chain variable region intergenic sequences (e.g., κ variable region intergenic sequences and/or λ variable region intergenic sequences). In some embodiments, the CAR variable gene locus includes non-human (e.g., rodent, rat, mouse) Ig light chain variable region intergenic sequences (e.g., κ variable region intergenic sequences and/or λ variable region intergenic sequences). In some embodiments, the CAR variable gene locus includes human or non-human (e.g., rodent, rat, mouse) TCRα variable region intergenic sequences. In some embodiments, the CAR gene locus comprises non-human regulatory elements (e.g., non-human promoters and/or enhancers. In some embodiments, the non-human regulatory elements are rodent regulatory elements (e.g., rat or mouse promoters or enhancers).

In some embodiments, the CAR variable region locus is a rearranged variable region locus comprising a Ig heavy chain variable region gene (a universal heavy chain variable region). In some embodiments, the rearranged Ig heavy chain variable region gene is a human rearranged Ig heavy chain variable region gene. Use of universal heavy chain variable regions facilitate the generation of bispecific antibodies in which at least one antigen-binding domain has specificity for a peptide/MHC complex. Exemplary rearranged Ig heavy chain variable regions are provided in U.S. Patent Pub. No. 2014/0245468, which is hereby incorporated by reference.

In some embodiments, the CAR variable region locus is a rearranged variable region locus comprising a Ig light chain variable region gene (a universal light chain variable region). In some embodiments, the rearranged Ig light chain variable region gene is a human rearranged Ig light chain variable region gene. Use of universal light chain variable regions facilitate the generation of bispecific antibodies in which at least one antigen-binding domain has binding specificity for a peptide/MHC complex. Exemplary rearranged Ig heavy chain variable regions are provided in U.S. Patent Pub. No. 2013/0185821, which is hereby incorporated by reference.

In certain embodiments, the CAR constant region locus comprises a TCRα or a TCRβ constant region gene. In some embodiments, the CAR constant region locus further comprises immunoglobulin regulatory sequences (e.g., regulatory sequences of human or endogenous species origin). In some embodiments, the CAR constant region locus comprises a mouse or rat IgM enhancer (Eμ) upstream of the TCRβ C2. In some embodiments, the TCR constant region gene also includes Ig constant region sequence. For example, in some embodiments, the CAR constant region locus includes a TCRβ constant region gene that includes nucleic acid sequence encoding for a Ig heavy chain CH1 domain. In some embodiments, the CAR constant region locus includes a TCRα constant region gene that includes nucleic acid sequence encoding for an Ig λ or Ig κ constant region or a portion thereof.

### Humanized MHC

In some embodiments, the genetically modified non-human animals and ES cells described herein express and/or comprise in their genome loci encoding humanized MHC class I α chain polypeptides (e.g., humanized HLA-A, HLA-B, HLA-C, HLA-E, HLA-F, HLA-g, HLA-K and/or HLA-L). In some embodiments, the humanized MHC class I α chain polypeptide is fully human. In some embodiments, the humanized MHC class I α chain polypeptide comprises a human extracellular domain (e.g., a human α1, α2, and α3 domains) and a cytoplasmic domain of endogenous species origin. Humanized MHC class I α chain polypeptides, loci encoding humanized MHC class I α chain polypeptides and non-human animals expressing humanized MHC class I α chain polypeptides are described in U.S. Pat. Pub. Nos. 2013/0111617, 2013/0185819 and 2014/0245467, each of which is incorporated by reference herein.

In some embodiments, the genetically modified non-human animals and ES cells described herein express and/or comprise in their genome a locus encoding humanized β-2-microglobulin polypeptide. Humanized β-2-microglobulin polypeptides, loci encoding humanized β-2-microglobulin polypeptides and non-human animals expressing humanized β-2-microglobulin polypeptides are described in U.S. Pat. Pub. Nos. 2013/0111617 and 2013/0185819, each of which is incorporated by reference herein.

In some embodiments, the genetically modified non-human animals and ES cells described herein express and/or comprise in their genome a loci encoding humanized MHC class II α chain polypeptides (e.g., humanized HLA-DMA, HLA-DOA, HLA-DPA, HLA-DQA and/or HLA-DRA). In some embodiments, the humanized MHC class II α chain polypeptide is fully human. In some embodiments, the humanized MHC class II α chain polypeptide comprises a human extracellular domain and a cytoplasmic domain of endogenous species origin. Humanized MHC class II α chain polypeptides, loci encoding humanized MHC class II α chain polypeptides and non-human animals expressing humanized MHC class II α chain polypeptides are described in U.S. Pat. Nos. 8,847,005 and 9,043,996 and U.S. Pat. Pub. No. 2014/0245467, each of which is incorporated by reference herein.

In some embodiments, the genetically modified non-human animals and ES cells described herein express and/or comprise in their genome a loci encoding humanized MHC class II β chain polypeptides (e.g., humanized HLA-DMB, HLA-DOB, HLA-DPB, HLA-DQB and/or HLA-DRB). In some embodiments, the humanized MHC class II β chain polypeptide is fully human. In some embodiments, the humanized MHC class II β chain polypeptide comprises a human extracellular domain and a cytoplasmic domain of endogenous species origin. Humanized MHC class II β chain polypeptides, loci encoding humanized MHC class II β chain polypeptides and non-human animals expressing humanized MHC class II β chain polypeptides are described in U.S. Pat. Nos. 8,847,005 and 9,043,996 and U.S. Pat. Pub. No. 2014/0245467, each of which is incorporated by reference herein.

Genetically modified non-human animals comprising CAR loci and humanized MHC I and/or MHC II (MHC IIα/IIβ) loci can be generated by breeding using conventional methods; alternatively, they can be generated by homologous recombination in ES cells already comprising one or more genetically engineered loci (e.g., CAR loci), and generating a non-human animal from said ES cells.

### Humanized CD4 and CD8 Receptors

In some embodiments, the genetically modified non-human animals and ES cells described herein express and/or comprise in their genome a locus encoding a humanized CD8 α chain polypeptide. In some embodiments, the humanized CD8 α chain polypeptide is fully human. In some embodiments, the humanized CD8 α chain polypeptide comprises a human extracellular immunoglobulin domain and a cytoplasmic domain of endogenous species origin. Humanized CD8 α chain polypeptides, loci encoding humanized CD8 α chain polypeptides and non-human animals expressing humanized CD8 α chain polypeptides are described in U.S. Pat. Pub. Nos. 2014/0245466 which is incorporated by reference herein.

In some embodiments, the genetically modified non-human animals and ES cells described herein express and/or comprise in their genome a locus encoding a humanized CD8 β chain polypeptide. In some embodiments, the humanized CD8 β chain polypeptide is fully human. In some embodiments, the humanized CD8 β chain polypeptide comprises a human extracellular immunoglobulin domain and a cytoplasmic domain of endogenous species origin. Humanized CD8 β chain polypeptides, loci encoding humanized CD8 β chain polypeptides and non-human animals expressing humanized CD8 β chain polypeptides are described in U.S. Pat. Pub. Nos. 2014/0245466 which is incorporated by reference herein.

In some embodiments, the genetically modified non-human animals and ES cells described herein express and/or comprise in their genome a locus encoding a humanized CD4 polypeptide. In some embodiments, the humanized CD4 polypeptide is fully human. In some embodiments, the humanized CD4 polypeptide comprises at least one human extracellular immunoglobulin domain and a cytoplasmic domain of endogenous species origin. In some embodiments, the humanized CD4 polypeptide comprises at least a human D1 immunoglobulin domain, a human D2 immunoglobulin domain, and a human D3 immunoglobulin domain, and a cytoplasmic domain of endogenous species origin. In some embodiments, the humanized CD4 polypeptide comprises a human D1 immunoglobulin domain, a human D2 immunoglobulin domain, a human D3 immunoglobulin domain, a D4 immunoglobulin domain of endogenous species origin and a cytoplasmic domain of endogenous species origin. Humanized CD4 polypeptides, loci encoding humanized CD4 polypeptides and non-human animals expressing humanized CD4 polypeptides are described in U.S. Pat. Pub. Nos. 2014/0245466 which is incorporated by reference herein.

Genetically modified non-human animals comprising CAR loci and humanized CD4 and/or CD8 (CD8α/CD8β) loci can be generated by breeding using conventional methods; alternatively, they can be generated by homologous recombination in ES cells already comprising one or more genetically engineered loci (e.g., CAR loci), and generating a non-human animal from said ES cells.

### Genetically Modified Non-Human Animals and ES Cells

In certain aspects, provided herein are genetically modified non-human animals that express a CAR and/or a CAR peptide as well as genetically modified non-human animal ES cells useful in the making of such non-human animals.

In certain aspects, provided herein are genetically modified non-human animals and non-human animal ES cells comprising in their germline and/or genome a CAR locus described herein. In some embodiments, the non-human animals or ES cells comprise two CAR loci in their germline and/or genome. In some embodiments, one locus comprises a TCRα constant region gene and one locus comprises a TCRβ constant region gene. In some embodiments, the CAR locus is located at an endogenous TCR locus.

In some embodiments, the non-human animal can be any non-human animal. In some embodiments, the non-human animal is a vertebrate. In some embodiments, the non-human animal is a mammal. In some embodiments, the genetically modified non-human animal described herein may be selected from a group consisting of a mouse, rat, rabbit, pig, bovine (e.g., cow, bull, buffalo), deer, sheep, goat, llama, chicken, cat, dog, ferret, primate (e.g., marmoset, rhesus monkey). For non-human animals where suitable genetically modifiable ES cells are not readily available, other methods can be employed to make a non-human animal comprising the genetic modifications described herein. Such methods include, for example, modifying a non-ES cell genome (e.g., a fibroblast or an induced pluripotent cell) and employing nuclear transfer to transfer the modified genome to a suitable cell, such as an oocyte, and gestating the modified cell (e.g., the modified oocyte) in a non-human animal under suitable conditions to form an embryo.

In some embodiments, the non-human animal is a mammal. In some embodiments, the non-human animal is a small mammal, e.g., of the superfamily Dipodoidea or Muroidea. In some embodiments, the non-human animal is a rodent. In certain embodiments, the rodent is a mouse, a rat or a hamster. In some embodiments, the rodent is selected from the superfamily Muroidea. In some embodiments, the non-human animal is from a family selected from Calomyscidae (e.g., mouse-like hamsters), Cricetidae (e.g., hamster, New World rats and mice, voles), Muridae (e.g., true mice and rats, gerbils, spiny mice, crested rats), Nesomyidae (e.g., climbing mice, rock mice, white-tailed rats, Malagasy rats and mice), Platacanthomyidae (e.g., spiny dormice), and Spalacidae (e.g., mole rates, bamboo rats, and zokors). In some embodiments, the rodent is selected from a true mouse or rat (family Muridae), a gerbil, a spiny mouse, and a crested rat. In some embodiments, the mouse is from a member of the family Muridae. In some embodiments, the non-human animal is a rodent. In some embodiments, the rodent is selected from a mouse and a rat. In some embodiments, the non-human animal is a mouse.

In some embodiments, the non-human animal is a mouse of a C57BL strain. In some embodiments, the C57BL strain is selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the non-human animal is a mouse of a 129 strain. In some embodiments, the 129 strain is selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. In some embodiments, the genetically modified mouse is a mix of a 129 strain and a C57BL strain. In some embodiments, the mouse is a mix of 129 strains and/or a mix of C57BL/6 strains. In some embodiments, the 129 strain of the mix is a 129S6 (129/SvEvTac) strain. In some embodiments, the mouse is a BALB strain (e.g., BALB/c). In some embodiments, the mouse is a mix of a BALB strain and another strain (e.g., a C57BL strain and/or a 129 strain). In some embodiments, the non-human animals provided herein can be a mouse derived from any combination of the aforementioned strains.

In some embodiments, the non-human animal provided herein is a rat. In some embodiments, the rat is selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

In certain embodiments, the genetically modified non-human animals or ES cells comprise in their genome and/or germline CAR loci, a humanized MHC class I α chain locus, a humanized β-2-microglobulin locus, a humanized MHC class II α chain locus, a humanized MHC class II β chain locus, a humanized CD8 α chain locus, a humanized CD8 β chain locus and/or a humanized CD4 locus. In some embodiments, the humanized MHC class I α chain locus is located at an endogenous MHC class I α chain locus. In some embodiment, the humanized β-2-microglobulin locus is located at an endogenous β-2-microglobulin locus. In some embodiments, the humanized MHC class II α chain locus is located at an endogenous MHC class II α chain locus. In some embodiments, the humanized MHC class II β chain locus is located at an endogenous MHC class II β chain locus. In some embodiments the humanized CD8 α chain locus is located at an endogenous CD8 α chain locus. In some embodiments, the humanized CD8 β chain locus is located at an endogenous CD8 β chain locus. In some embodiments the humanized CD4 locus is located at an endogenous CD4 locus. In some embodiments, the genetically modified non-human animal does not express endogenous MHC class I α chain polypeptides, endogenous β-2-microglobulin polypeptides, endogenous MHC class II α chain polypeptides, endogenous MHC class II β chain polypeptides, endogenous CD8 α chain polypeptides, endogenous CD8 β chain polypeptides and/or endogenous CD4 polypeptides. Such animals are described in, for example, U.S. Pat. Pub. Nos. 2013/0111617, 2013/0185819, 2014/0245466 and 2014/0245467, and U.S. Pat. Nos. 8,847,005 and 9,043,996, each of which is incorporated by reference herein.

In certain aspects, the genetically modified non-human animal expresses a CAR polypeptide described herein. In some embodiments, the genetically modified non-human animal expresses a CAR comprising two CAR polypeptides. In certain embodiments, the CAR has binding specificity for a peptide/MHC complex. In some embodiments, the CAR is expressed on T cells (e.g., CD4 T cells or CD8 T cells) in the non-human animal. In some embodiments, the non-human animal does not express an αβ TCR. In some embodiments, the CAR expressing T cells undergo positive selection during T cell development. In some embodiments, the CAR expressing T cells undergo negative selection during T cell development.

The genetically modified non-human animals and ES cells can be generated using any appropriate method known in the art. For example, such genetically modified non-human animal ES cells can be generated using VELOCIGENE® technology, which is described in U.S. Pat. Nos. 6,586,251, 6,596,541, 7,105,348, and Valenzuela et al. (2003) “High-throughput engineering of the mouse genome coupled with high-resolution expression analysis” Nat. Biotech. 21(6): 652-659, each of which is hereby incorporated by reference. Modifications can also be made using a genome targeted nuclease system, such as a CRISPR/Cas system, a transcription activator-like effector nuclease (TALEN) system or a zinc finger nuclease (ZFN) system. In some embodiments, modifications are made using a CRISPR/Cas system, as described, for example, in U.S. patent application Ser. Nos. 14/314,866, 14/515,503, 14/747,461 and 14/731,914, each of which is incorporated by reference. In some embodiments, variable region gene segments are serially added to a CAR locus through a series of targeting events in which large targeting vectors are sequentially added to an expanding CAR locus one after another. In some embodiments, multiple large targeting vectors (e.g., two or more) are simultaneously incorporated into the CAR locus in a single targeting event (e.g., a double-targeting event). Exemplary methods of making such genetically modified non-human animals and ES cells are provided herein in Example 2.

ES cells described herein can then be used to generate a non-human animal using methods known in the art. For example, the mouse non-human animal ES cells described herein can be used to generate genetically modified mice using the VELOCIMOUSE® method, as described in U.S. Pat. No. 7,294,754 and Poueymirou et al., Nature Biotech 25:91-99 (2007), each of which is hereby incorporated by reference. Resulting mice can be bread to homozygosity.

### Methods of Using the Genetically Modified Non-Human Animals

The genetically modified non-human animals described herein can be used in any process for which an animal expressing a CAR might be useful. For example such non-human animals can be used to make CARs, to make a T cells expressing CARs, to make T cell hybridomas expressing CARs, to make nucleic acids encoding rearranged Ig variable regions, and to make antibodies or antibody fragments.

In certain embodiments of the methods described herein include the immunization of a transgenic non-human animal in order to induce a T cell immune response against a peptide/MHC complex. In some embodiments a genetically modified non-human animal described herein is exposed to an antigen comprising a peptide such that the peptide is presented on a MHC in the non-human animal.

In some embodiments, any method of exposing the genetically modified non-human animal described herein to an antigen comprising a peptide such that the peptide is presented on a MHC in the non-human animal such that a T cell response to the peptide is induced in the animal can be used.

In some embodiments, the MHC on which the peptide is presented is a class I MHC. In some embodiments, the class I MHC is HLA-A, HLA-B, HLA-C, HLA-E, HLA-F or HLA-G. In some embodiments, the peptide is 8-10 amino acids in length.

In some embodiments, the MHC on which the peptide is presented is a class II MHC. In some embodiments, the class II MHC is HLA-DM, HLA-DO, HLA-DP, HLA-DQ or HLA-DR. In some embodiments, the peptide is 10-25 amino acids in length. In some embodiments, the peptide is 13-25 amino acids in length. In some embodiments, the peptide is 15-18 amino acids in length.

In some embodiments, the peptide comprises an epitope of a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pme117, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a. In some embodiments, the antigen is a neo-antigen.

In some embodiment, the peptide comprises an epitope of an antigen expressed by an infectious pathogen. In some embodiments, the pathogen is a virus, a bacteria, a fungus, a helminth, or a protozoa For example, in some embodiments, the virus is HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. In some embodiments, the parasite is malaria. In some embodiments, pathogen is Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum.

In some embodiments, the peptide comprises an epitope of an protein that is the target of an autoreactive T cell in an inflammatory disease, skin or organ transplantation rejection, graft-versus-host disease (GVHD), or autoimmune diseases. Examples of autoimmune diseases include, for example, glomerular nephritis, arthritis, dilated cardiomyopathy-like disease, ulceous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis. Exemplary proteins include targeted by autoreactive T cells include, for example, p205, insulin, thyroid-stimulating hormone, tyrosinase, TRP1, and myelin.

In some embodiments, the genetically modified non-human animal is exposed to the peptide by administering to the non-human animal with a virus (e.g., a retrovirus, an adenovirus, a vaccinia virus or a lentivirus) comprising a nucleic acid sequence encoding the peptide. Methods for viral vaccination are provided, for example, in U.S. Pat. Nos. 6,001,349, 8,663,622, 8,691,502, 8,377,688, as well as Precopio et al., JEM 204:1405-1416 (2007), each of which is hereby incorporated by reference in its entirety. In some embodiments, the non-human animal is administered the virus directly, such that the non-human animal processes the antigen and presents it on its MHC. In some embodiments, a cell (e.g., an antigen presenting cell, such as a dendritic cell) is infected with the virus in vitro or ex vivo which is then administered to the non-human animal. In some embodiments, the virus encodes a peptide/MHC complex (e.g., a single-chain peptide/MHC complex). Examples of single-chain peptide/MHC based vaccines are provided in Truscott et al., J. Immunol. 178:6280-6289 (2007), EP1773383, Kim et al., Vaccine 30:2178-2186 (2012), Kim et al., J. Immunol. 184:4423-4430 (2010), each of which are hereby incorporated by reference.

In some embodiments, the genetically modified non-human animal is exposed to the peptide by administering to the animal a nucleic acid encoding the peptide such that the peptide is expressed in the non-human animal. In some embodiments, the non-human animal is administered a nucleic acid encoding a single chain peptide/MHC complex. Examples of single-chain peptide/MHC based vaccines are provided in Truscott et al., J. Immunol. 178:6280-6289 (2007), EP1773383, Kim et al., Vaccine 30:2178-2186 (2012), Kim et al., J. Immunol. 184:4423-4430 (2010), each of which are hereby incorporated by reference. In certain embodiments, the nucleic acid is a DNA vector. The delivery of nucleic acids can be by any technique known in the art including viral mediated gene transfer and liposome mediated gene transfer. A polynucleotide of interest is associated with a liposome to form a gene delivery vehicle as described in, for example, U.S. Pat. Nos. 6,770,291, 7,001,614, 6,749,863, 5,512,295 and 7,112,338, each of which is hereby incorporated by reference. In some embodiments, the nucleic acid is an mRNA vector. Exemplary methods for generating and administering mRNA vectors are described in, for example, U.S. Pat. No. 8,278,036 and U.S. Pat. Pub. Nos. 2013/151736 and 2012/135805, each of which is hereby incorporated by reference.

In some embodiments, the genetically modified non-human animal is exposed to the peptide by administering to the genetically modified non-human animal a peptide/MHC complex. In some embodiments, the non-human animal is administered a single chain peptide/MHC complex (e.g., a single chain ecto-MHC/β-2-microglobulin/peptide protein complex). In some embodiments, the peptide/MHC complex is administered as a multimer (e.g., dimer, a trimer, a tetramer). In some embodiments, the peptide/MHC complex is present on the surface of a cell. Exemplary methods for generating and administering peptide/MHC complexes are provided in U.S. Pat. Nos. 6,045,796, 5,869,270 and 7,141,656, as well as Truscott et al., J. Immunol. 178:6280-6289 (2007), EP1773383, Kim et al., Vaccine 30:2178-2186 (2012), Kim et al., J. Immunol. 184:4423-4430 (2010) and Livingstone Methods: A Companion to Methods in Enzymology 9:422-429 (1996), each of which is hereby incorporated by reference.

In some embodiments of the methods described herein, the method includes the step of obtaining a T cell expressing a CAR specific for the peptide/MHC complex from the genetically modified non-human animal. In certain embodiments, any method known in the art can be used to obtain such T cells. For example, such T cells can be obtained from the spleen, lymph nodes and/or peripheral blood of the animal. Such T cells can be screened for binding specificity using methods available in the art. For example, cells expressing a CAR specific for a specific peptide/MHC complex can be purified using peptide MHC complex loaded onto a solid support, such as a column or beads, such as magnetic beads, or labeled peptide/MHC can be used to stain such T cells, which then can be purified using fluorescence-activated cell sorting (FACS) and/or magnetic-activated cell sorting (MACS).

In some embodiments, the methods described herein include the step of making a T cell hybridoma from a T cell. Methods useful for making a T cell hybridoma are known in the art and described, for example, in Hedrick et al., Cell 30:141-152 (1982) and Kruisbeek Curr. Protoc. Immunol. Chapter 3 (2001) and White et al., Methods in Molecular Biology 134:185-193 (2000), each of which is hereby incorporated by reference.

In some embodiments, the methods provided herein include the step of isolating a nucleic acid encoding an Ig variable domain of a CAR from a T cell. In some embodiments of the methods described herein, any method can be used to isolate the nucleic acid comprising encoding the Ig variable domain.

In some embodiments, the step of isolating the nucleic acid comprises making a T cell hybridoma from the T cell and isolating the nucleic acid from the T cell hybridoma. In some embodiments, the nucleic acid is isolated using a nucleic acid amplification process. For example, in some embodiments the nucleic acid amplification process is polymerase chain reaction (PCR), ligase chain reaction (LCR), strand displacement amplification (SDA), transcription mediated amplification (TMA), self-sustained sequence replication (3SR), Qβ replicase based amplification, nucleic acid sequence-based amplification (NASBA), repair chain reaction (RCR), boomerang DNA amplification (BDA) or rolling circle amplification (RCA).

In some embodiments, the nucleic acid is isolated by sequencing the rearranged Ig variable region gene in a CAR locus of the T cell or the T cell hybridoma and synthesizing a nucleic acid sequence comprising the rearranged Ig variable region gene. Exemplary nucleic acid sequencing processes include, but are not limited to chain termination sequencing, sequencing by ligation, sequencing by synthesis, pyrosequencing, ion semiconductor sequencing, single-molecule real-time sequencing, 454 sequencing, and/or Dilute-‘N’-Go sequencing.

Once DNA fragments encoding a heavy and light chain Ig variable region segments are obtained, these DNA fragments can be further manipulated by standard recombinant DNA techniques, for example to convert the variable region genes to full-length antibody chain genes, to Fab fragment genes or to a scFv gene. In these manipulations, a variable domain-encoding DNA fragment is operatively linked to another DNA fragment encoding another protein, such as an antibody constant domain or a flexible linker. The term “operatively linked”, as used in this context, is intended to mean that the two DNA fragments are joined such that the amino acid sequences encoded by the two DNA fragments remain in-frame.

The isolated DNA encoding the heavy chain variable domain can be converted to a full-length heavy chain gene by operatively linking the variable domain-encoding DNA to another DNA molecule encoding heavy chain constant domains (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant domain can be an IgG1, IgG2, IgG3, IgG4, IgA, IgE, IgM or IgD constant domain, but most preferably is an IgG1 or IgG4 constant domain. For a Fab fragment heavy chain gene, the V_{H}-encoding DNA can be operatively linked to another DNA molecule encoding only the heavy chain CH1 constant domain.

The isolated DNA encoding the light chain Ig variable domain can be converted to a full-length light chain gene (as well as a Fab light chain gene) by operatively linking the variable domain-encoding DNA to another DNA molecule encoding the light chain constant domain, such as a κ or λ constant domain. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

Thus, in some embodiments, the methods described herein include the step of operably linking a nucleic acid sequence encoding a heavy chain Ig variable domain with a nucleic acid sequence encoding a heavy chain Ig constant domain in a host cell such that the host cell expresses an Ig heavy chain polypeptide comprising the Ig heavy chain variable domain and the Ig heavy chain constant domain. In some embodiments, the method includes the step of operably linking a nucleic acid sequence encoding a light chain Ig variable domain with a nucleic acid sequence encoding a light chain Ig constant domain in a host cell such that the host cell expresses an Ig light chain polypeptide comprising the Ig light chain variable domain and the Ig heavy chain constant domain. In some embodiments, the method includes the step of operably linking a nucleic acid sequence encoding a heavy chain Ig variable domain with a nucleic acid sequence encoding a heavy chain Ig constant domain in a host cell and operably linking a nucleic acid sequence encoding a light chain Ig variable domain with a nucleic acid sequence encoding a light chain Ig constant domain in the host cell such that the host cell expresses an antibody having a heavy chain comprising the heavy chain Ig variable domain and the heavy chain Ig constant domain and a light chain comprising the light chain Ig variable domain and the light chain Ig constant domain. Ig variable regions can be linked with Ig constant regions using standard molecular biology techniques well known in the art. In some embodiments, any host cell capable of expressing an immunoglobulin polypeptide can be used. In some embodiments the cell is a CHO cell, a HEK-293 cell, a BHK cell, a NS0 cell, a SP2/0 cell or a Vero cell.

In some embodiments, the methods provided herein include the step of operably linking a nucleic acid sequence encoding a heavy chain Ig variable domain with a nucleic acid sequence encoding a TCR constant domain (e.g., a TCRβ constant domain or a TCRα constant domain) in a cell (e.g., a human cell, such as a human T cell) such that the cell expresses a CAR polypeptide comprising the Ig heavy chain variable domain and the TCR constant domain. In some embodiments, the methods include operably linking a nucleic acid sequence encoding a light chain Ig variable domain with a nucleic acid sequence encoding a TCR constant domain (e.g., a TCRβ constant domain or a TCRα constant domain) in a cell (e.g., a human cell, such as a human T cell) such that the cell expresses a CAR polypeptide comprising the Ig light chain variable domain and the TCR constant domain. In some embodiments, the methods include operably linking a nucleic acid sequence encoding a heavy chain Ig variable domain and a first TCR constant domain (e.g., a TCRβ constant domain or a TCRα constant domain) in a cell (e.g., a human cell, such as a human T cell) and operably linking a nucleic acid sequence encoding a light chain Ig variable domain and a nucleic acid sequence encoding a second TCR constant domain (e.g., a TCRβ constant domain if the first TCR constant domain is a TCRα constant domain or a TCRα constant domain if the first TCR constant domain is a TCRβ constant domain) in the cell such that the cell expresses an CAR having a first CAR chain polypeptide comprising the heavy chain Ig variable domain and the first TCR constant domain and a second CAR polypeptide comprising the light chain Ig variable domain and the second TCR constant domain. In some embodiments, the TCR constant domains are human TCR constant domains. Ig variable regions can be linked with TCR constant regions using standard molecular biology techniques well known in the art. In some embodiments, the cell is an ex-vivo cell (e.g., an ex vivo human cell, such as an ex vivo human T cell) isolated from a subject.

### Antibodies

In certain aspects, provided herein are antibodies that have binding specificity for a peptide/MHC complex (e.g., a peptide/class I MHC complex or a peptide/class II MHC complex). In some embodiments, the antibodies are fully human. In some embodiments, the CARs are obtainable and/or obtained according to a method described herein (e.g., using a non-human animal comprising CAR loci as described herein).

In certain embodiments, the antibodies and antibody fragments provided herein are able to specifically bind a peptide/MHC complex with a dissociation constant of no greater than 10⁻⁶, 10⁻⁷, 10⁻⁸ or 10⁻⁹ M. In some embodiments, the binding affinity of the antibody to a peptide/MHC complex (as expressed by K_{D}) is at least 10 fold less, at least 100 fold less or at least 1000 fold less than the affinity of the antibody for the peptide for the same MHC protein presenting an unrelated peptide. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis.

In some embodiments, the antibody is specific for a peptide/MHC class I complex. In some embodiments, the MHC class I is HLA-A, HLA-B, HLA-C, HLA-E, HLA-F or HLA-G. In some embodiments, the peptide is 8-10 amino acids in length.

In some embodiments, the antibody is specific for a peptide/MHC class I complex. In some embodiments, the MHC class II is HLA-DM, HLA-DO, HLA-DP, HLA-DQ or HLA-DR. In some embodiments, the peptide is 10-25 amino acids in length. In some embodiments, the peptide is 13-25 amino acids in length. In some embodiments, the peptide is 15-18 amino acids in length.

In some embodiments, the peptide comprises an epitope of a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pme117, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, MEL Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a. In some embodiments, the antigen is a neo-antigen.

In some embodiment, the peptide comprises an epitope of an antigen expressed by an infectious pathogen. In some embodiments, the pathogen is a virus, a bacteria, a fungus, a helminth, or a protozoa For example, in some embodiments, the virus is HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. In some embodiments, the parasite is malaria. In some embodiments, pathogen is Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum.

In some embodiments, the peptide comprises an epitope of an protein that is the target of an autoreactive T cell in an inflammatory disease, skin or organ transplantation rejection, graft-versus-host disease (GVHD), or autoimmune diseases. Examples of autoimmune diseases include, for example, glomerular nephritis, arthritis, dilated cardiomyopathy-like disease, ulceous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis. Exemplary proteins include targeted by autoreactive T cells include, for example, p205, insulin, thyroid-stimulating hormone, tyrosinase, TRP1, and myelin.

In some embodiments, the antibodies provided herein comprise human heavy chain variable domains. In some embodiments, the antibodies comprise human heavy chain constant domains. In some embodiments, the antibodies provided herein comprise a IgG1, IgG2, IgG3, IgG4, IgA, IgE, IgM or IgD constant domain. The sequences of human heavy chain constant domains are known in the art (see e.g., Kabat, E. A., et al. (1991) Sequences of Proteins of Immunological Interest, Fifth Edition, U.S. Department of Health and Human Services, NIH Publication No. 91-3242). In some embodiments, the antibodies provided herein lack a heavy chain constant domain or a portion thereof.

In some embodiments, the antibodies provided herein comprises a modified Fc domain (e.g., a mutation that alters the interaction between the Fc and a Fc receptor). For example, in some embodiments, the antibodies provided herein comprise modification to their Fc domain at position 235, 236, 237, 239, 265, 267, 268, 269, 270, 298, 326, 327, 330, 332, 350, 351, 366, 392, 394, 405 and/or 407 (using the EU numbering system). In some embodiments, the modification is selected from the group consisting of L235A, G236E, G237F, S239E, S239D, D265E, D265S, S267E, S267D, S267G, H268E, H268D, E269L, D270N, D270E, S298A, K326A, K326D, A327H, A327V, A327L, A330I, A330S, 1332E, T350V, L351Y, T366L, K392M, K392L, T394W, F405A and/or Y407V (using the EU numbering system). In some embodiments, the antibodies comprise multiple modifications to their Fc domain. In some embodiments, the multiple modifications are selected from the group consisting of D270N/K326D, S239E/S298A/K326A/A327H, L235A/S239E/D265E/A327H, G236E/G237F/S239E, G237F/S239E/D265E, G327F/S239E/H268D, G236E/D270N/A327V/1332E, G237F/S239E/A327H, G237F/A327L/A330I, S239D/D265S/S298A/I332E, S239E/D265S/H268D/I332E, S239E/D265S/I332E, S239E/S267E/H268D, S239E/A327L/A330I, D265E/S267D/A330S, S267G/H268E/D270E, H268D/E269L/S298A/K326A/A327H, H268D//K326A/A327H. Additional Fc modifications and combinations of Fc modifications are provided in U.S. Pat. Nos. 5,624,821, 5,648,260, 6,528,624, 6,737,056, 7,122,637, 7,183,387, 7,297,775, 7,317,091, 7,332,581, 7,632,497, 7,662,925, 7,695,936, 8,093,359, 8,216,805, 8,218,805, 8,388,955 and 8,937,158, and U.S. Patent Publication Nos. 2005/0054832, 2006/0222653, 2006/0275282, 2006/0275283, 2007/0190063, 2008/0154025, 2009/0042291 2013/0108623 and 2013/0089541, each of which is hereby incorporated by reference.

In some embodiments, the antibody is a bi-specific antibody. In some embodiments, In some embodiments, the two antigen binding domains of the bi-specific antibody have distinct heavy chain variable domains but have identical light chain variable domains. In some embodiments, the Fc domains of the heavy chains comprise modifications to facilitate heavy chain heterodimer formation and/or to inhibit heavy chain homodimer formation. Such modifications are provided, for example, in U.S. Pat. Nos. 5,731,168, 5,807,706, 5,821,333, 7,642,228 and 8,679,785 and in U.S. Pat. Pub. No. 2013/0195849, each of which is hereby incorporated by reference.

In some embodiments, the antibodies provided herein have human light chain variable domains. In some embodiments, the light chain variable domains are λ light chain variable domains. In some embodiments, the light chain variable domains are κ light chain variable domains. In some embodiments, the antibodies have human light chain constant domains. In some embodiments, the light chain constant domains are λ light chain constant domains. In some embodiments, the light chain constant domains are κ light chain constant domains. The sequences of human light chain constant domains are known in the art (see e.g., Kabat, E. A., et al. (1991) Sequences of Proteins of Immunological Interest, Fifth Edition, U.S. Department of Health and Human Services, NIH Publication No. 91-3242)

In some embodiments, the antibodies described herein are intact antibodies. In some embodiments, the antibodies described herein are antibody fragment that retain antigen binding. In some embodiments, the antibody fragment is a Fab, Fab′, F(ab′)₂, Fv, scFv, disulfide linked Fv, Fd, single-chain antibodies, isolated CDRH3 or another antibody fragment that retain at least a portion of the variable domain of an intact antibody.

### Chimeric Antigen Receptors

In certain aspects, provided herein are chimeric antigen receptors (CARs) having binding specificity for a peptide/MHC complex (e.g., a peptide/class I MHC complex or a peptide/class II MHC complex). In some embodiments, the CARs are fully human. In some embodiments, the CARs are obtainable and/or obtained according to a method described herein (e.g., using a non-human animal comprising CAR loci as described herein).

In some embodiments, the CAR binds to the peptide/MHC complex with an affinity corresponding to a K_{D} of less than 10⁻⁷ M, 10⁻⁸ M or 10⁻⁹ M. In some embodiments, the binding affinity of the CAR to a peptide/MHC complex (as expressed by K_{D}) is at least 10 fold less, at least 100 fold less or at least 1000 fold less than the affinity of the CAR for the peptide when not presented by the MHC. In some embodiments, the binding affinity of the CAR to a peptide/MHC complex (as expressed by K_{D}) is at least 10 fold less, at least 100 fold less or at least 1000 fold less than the affinity of the CAR for the peptide for the same MHC protein presenting an unrelated peptide. Standard assays to evaluate the binding ability of CARs are known in the art, including for example, ELISAs, Western blots and RIAs. The binding kinetics (e.g., binding affinity) of the CARs also can be assessed by standard assays known in the art, such as by Biacore analysis.

In some embodiments, the CAR is specific for a peptide/MHC class I complex. In some embodiments, the MHC class I is HLA-A, HLA-B, HLA-C, HLA-E, HLA-F or HLA-G. In some embodiments, the peptide is 8-10 amino acids in length.

In some embodiments, the CAR is specific for a peptide/MHC class I complex. In some embodiments, the MHC class II is HLA-DM, HLA-DO, HLA-DP, HLA-DQ or HLA-DR. In some embodiments, the peptide is 10-25 amino acids in length. In some embodiments, the peptide is 13-25 amino acids in length. In some embodiments, the peptide is 15-18 amino acids in length.

In some embodiments, the peptide comprises an epitope of a cancer-associated antigen. Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pme117, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, MEL Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a. In some embodiments, the antigen is a neo-antigen.

In some embodiment, the peptide comprises an epitope of an antigen expressed by an infectious pathogen. In some embodiments, the pathogen is a virus, a bacteria, a fungus, a helminth, or a protozoa For example, in some embodiments, the virus is HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. In some embodiments, the parasite is malaria. In some embodiments, pathogen is Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum.

In some embodiments, the peptide comprises an epitope of an protein that is the target of an autoreactive T cell in an inflammatory disease, skin or organ transplantation rejection, graft-versus-host disease (GVHD), or autoimmune diseases. Examples of autoimmune diseases include, for example, glomerular nephritis, arthritis, dilated cardiomyopathy-like disease, ulceous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis. Exemplary proteins include targeted by autoreactive T cells include, for example, p205, insulin, thyroid-stimulating hormone, tyrosinase, TRP1, and myelin.

In some embodiments, such CARs comprise a first CAR polypeptide comprising an Ig heavy chain variable domain and a TCRβ constant domain and a second CAR polypeptide comprising an Ig light chain variable domain (e.g., an Ig κ variable domain or an Ig λ variable domain) and a TCRα constant domain. In some embodiments, the Ig heavy chain variable domain and/or the Ig light chain variable domain are human Ig variable domains. In some embodiments, the TCRβ constant domain and/or the TCRα constant domain are non-human constant domains (e.g., rat or mouse constant domains). In some embodiments, the TCRβ constant domain and/or the TCRα constant domain are human constant domains. The Ig variable domains of the CAR can be generated using the methods described herein or using any other method known in the art. For example, an anti-peptide/MHC antibody can be generated using a method known in the art (e.g., using phage display or yeast display) and then a nucleic acid sequence encoding the variable domains of the antibody can be linked to TCR constant domain genes. Examples of antibodies having binding specificity for peptide/MHC complexes and methods for producing such antibodies are provided, for example, in U.S. Pat. Nos. 6,992,176, 7,718,777 and 8,815,528, as well as in Stewart-Jones et al., Proc. Nat'l. Acad. Sci. USA 106:5784-88 (2009) and Hulsmeyer et al., J. Biol. Chem. 280:2972-80 (2005), each of which is hereby incorporated by reference.

### Pharmaceutical Compositions

In certain embodiments, provided herein is a composition, e.g., a pharmaceutical composition, containing at least one agent described herein (e.g., an antibody described herein and/or a CAR described herein) formulated together with a pharmaceutically acceptable carrier.

The pharmaceutical compositions provided herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Pharmaceutical compositions provided herein suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In certain embodiments, the compositions comprise a an antibody and/or a CAR described herein in a concentration resulting in a w/v appropriate for a desired dose. The antibody and/or CAR may be present in the composition at a concentration of at least 1 mg/mL, at least 5 mg/mL, at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 75 mg/mL, at least 80 mg/mL, at least 85 mg/mL, at least 90 mg/mL, at least 95 mg/mL, at least 100 mg/mL, at least 105 mg/mL, at least 110 mg/mL, at least 115 mg/mL, at least 120 mg/mL, at least 125 mg/mL, at least 130 mg/mL, at least 135 mg/mL, at least 140 mg/mL, at least 150 mg/mL, at least 200 mg/mL, at least 250 mg/mL, or at least 300 mg/mL.

In some embodiments, the composition comprises one or more active compounds as necessary for the particular indication being treated, typically those with complementary activities that do not adversely affect each other. Such additional active compounds are suitably present in combination in amounts that are effective for the purpose intended.

In some embodiments, compositions are prepared by mixing an antibody and/or CAR described herein with optional physiologically acceptable carriers, excipients or stabilizers, including, but not limited to buffering agents, saccharides, salts, surfactants, solubilizers, polyols, diluents, binders, stabilizers, salts, lipophilic solvents, amino acids, chelators, preservatives, or the like (Goodman and Gilman's The Pharmacological Basis of Therapeutics, 12th edition, L. Brunton, et al. and Remington's Pharmaceutical Sciences, 16th edition, Osol, A. Ed. (1999)), in the form of lyophilized compositions or aqueous solutions at a desired final concentration. Acceptable carriers, excipients, or stabilizers are nontoxic to recipients at the dosages and concentrations employed, and include buffers such as histidine, phosphate, citrate, glycine, acetate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrolidone; amino acids such as glycine, glutamine, asparagine, histidine, arginine, or lysine; monosaccharides, disaccharides, and other carbohydrates including trehalose, glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, polysorbate 80, PLURONICS® or polyethylene glycol (PEG).

In some embodiments, the buffering agent is histidine, citrate, phosphate, glycine, or acetate. The saccharide excipient may be trehalose, sucrose, mannitol, maltose or raffinose. The surfactant may be polysorbate 20, polysorbate 40, polysorbate 80, or Pluronic F68. The salt may be NaCl, KCl, MgCl2, or CaCl2

In some embodiments, the composition comprises a buffering or pH adjusting agent to provide improved pH control. Such a composition may have a pH of between about 3.0 and about 9.0, between about 4.0 and about 8.0, between about 5.0 and about 8.0, between about 5.0 and about 7.0, between about 5.0 and about 6.5, between about 5.5 and about 8.0, between about 5.5 and about 7.0, or between about 5.5 and about 6.5. In a further embodiment, such a composition has a pH of about 3.0, about 3.5, about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.5, about 8.0, about 8.5, or about 9.0. In a specific embodiment, a composition has a pH of about 6.0. One of skill in the art understands that the pH of a composition generally should not be equal to the isoelectric point of the particular antibody or CAR to be used in the composition. Typically, the buffering agent is a salt prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In addition, amino acid components can also function in a buffering capacity. Representative amino acid components which may be utilized in the composition as buffering agents include, but are not limited to, glycine and histidine. In certain embodiments, the buffering agent is chosen from histidine, citrate, phosphate, glycine, and acetate. In a specific embodiment, the buffering agent is histidine. In another specific embodiment, the buffering agent is citrate. In yet another specific embodiment, the buffering agent is glycine. The purity of the buffering agent should be at least 98%, or at least 99%, or at least 99.5%. As used herein, the term “purity” in the context of histidine and glycine refers to chemical purity of histidine or glycine as understood in the art, e.g., as described in The Merck Index, 13th ed., O'Neil et al. ed. (Merck & Co., 2001).

In certain embodiments, the composition comprises histidine as a buffering agent. In certain embodiments the histidine is present in the composition at a concentration of at least about 1 mM, at least about 5 mM, at least about 10 mM, at least about 20 mM, at least about 30 mM, at least about 40 mM, at least about 50 mM, at least about 75 mM, at least about 100 mM, at least about 150 mM, or at least about 200 mM histidine. In another embodiment, a composition comprises between about 1 mM and about 200 mM, between about 1 mM and about 150 mM, between about 1 mM and about 100 mM, between about 1 mM and about 75 mM, between about 10 mM and about 200 mM, between about 10 mM and about 150 mM, between about 10 mM and about 100 mM, between about 10 mM and about 75 mM, between about 10 mM and about 50 mM, between about 10 mM and about 40 mM, between about 10 mM and about 30 mM, between about 20 mM and about 75 mM, between about 20 mM and about 50 mM, between about 20 mM and about 40 mM, or between about 20 mM and about 30 mM histidine. In a further embodiment, the composition comprises about 1 mM, about 5 mM, about 10 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 150 mM, or about 200 mM histidine. In a specific embodiment, a composition may comprise about 10 mM, about 25 mM, or no histidine.

In some embodiments, the composition comprises a carbohydrate excipient. Carbohydrate excipients can act, e.g., as viscosity enhancing agents, stabilizers, bulking agents, solubilizing agents, and/or the like. Carbohydrate excipients are generally present at between about 1% to about 99% by weight or volume, e.g., between about 0.1% to about 20%, between about 0.1% to about 15%, between about 0.1% to about 5%, between about 1% to about 20%, between about 5% to about 15%, between about 8% to about 10%, between about 10% and about 15%, between about 15% and about 20%, between 0.1% to 20%, between 5% to 15%, between 8% to 10%, between 10% and 15%, between 15% and 20%, between about 0.1% to about 5%, between about 5% to about 10%, or between about 15% to about 20%. In still other specific embodiments, the carbohydrate excipient is present at 1%, or at 1.5%, or at 2%, or at 2.5%, or at 3%, or at 4%, or at 5%, or at 10%, or at 15%, or at 20%.

In some embodiments, the composition comprises a carbohydrate excipient. Carbohydrate excipients suitable for use in the compositions include, but are not limited to, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In certain embodiments, the carbohydrate excipients for use in the compositions provided herein are chosen from sucrose, trehalose, lactose, mannitol, and raffinose. In a specific embodiment, the carbohydrate excipient is trehalose. In another specific embodiment, the carbohydrate excipient is mannitol. In yet another specific embodiment, the carbohydrate excipient is sucrose. In still another specific embodiment, the carbohydrate excipient is raffinose. The purity of the carbohydrate excipient should be at least 98%, or at least 99%, or at least 99.5%.

In some embodiments, the composition comprises trehalose. In certain embodiments, a composition comprises at least about 1%, at least about 2%, at least about 4%, at least about 8%, at least about 20%, at least about 30%, or at least about 40% trehalose. In another embodiment, a composition comprises between about 1% and about 40%, between about 1% and about 30%, between about 1% and about 20%, between about 2% and about 40%, between about 2% and about 30%, between about 2% and about 20%, between about 4% and about 40%, between about 4% and about 30%, or between about 4% and about 20% trehalose. In a further embodiment, a composition comprises about 1%, about 2%, about 4%, about 6%, about 8%, about 15%, about 20%, about 30%, or about 40% trehalose. In a specific embodiment, a composition comprises about 4%, about 6% or about 15% trehalose.

In certain embodiments, the composition comprises an excipient. In a specific embodiment, a composition comprises at least one excipient chosen from: sugar, salt, surfactant, amino acid, polyol, chelating agent, emulsifier and preservative. In certain embodiments, a composition comprises a salt, e.g., a salt selected from: NaCl, KCl, CaCl2, and MgCl2. In a specific embodiment, the composition comprises NaCl.

In some embodiments, the composition comprises an amino acid, e.g., lysine, arginine, glycine, histidine or an amino acid salt. The composition may comprise at least about 1 mM, at least about 10 mM, at least about 25 mM, at least about 50 mM, at least about 100 mM, at least about 150 mM, at least about 200 mM, at least about 250 mM, at least about 300 mM, at least about 350 mM, or at least about 400 mM of an amino acid. In another embodiment, the composition may comprise between about 1 mM and about 100 mM, between about 10 mM and about 150 mM, between about 25 mM and about 250 mM, between about 25 mM and about 300 mM, between about 25 mM and about 350 mM, between about 25 mM and about 400 mM, between about 50 mM and about 250 mM, between about 50 mM and about 300 mM, between about 50 mM and about 350 mM, between about 50 mM and about 400 mM, between about 100 mM and about 250 mM, between about 100 mM and about 300 mM, between about 100 mM and about 400 mM, between about 150 mM and about 250 mM, between about 150 mM and about 300 mM, or between about 150 mM and about 400 mM of an amino acid. In a further embodiment, a composition comprises about 1 mM, 1.6 mM, 25 mM, about 50 mM, about 100 mM, about 150 mM, about 200 mM, about 250 mM, about 300 mM, about 350 mM, or about 400 mM of an amino acid.

In some embodiments, the composition comprises a surfactant. The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. Pharmaceutically acceptable surfactants like polysorbates (e.g., polysorbates 20 or 80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA® series (Mona Industries, Inc., Paterson, N.J.), polyethyl glycol, polypropyl glycol, and copolymers of ethylene and propylene glycol (e.g., PLURONICS® PF68, etc.), can optionally be added to the compositions to reduce aggregation. In certain embodiments, a composition comprises Polysorbate 20, Polysorbate 40, Polysorbate 60, or Polysorbate 80. Surfactants are particularly useful if a pump or plastic container is used to administer the composition. The presence of a pharmaceutically acceptable surfactant mitigates the propensity for the protein to aggregate. The compositions may comprise a polysorbate which is at a concentration ranging from between about 0.001% to about 1%, or about 0.001% to about 0.1%, or about 0.01% to about 0.1%. In other specific embodiments, the compositions comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%.

In some embodiments, the composition comprises other excipients and/or additives including, but not limited to, diluents, binders, stabilizers, lipophilic solvents, preservatives, adjuvants, or the like. Pharmaceutically acceptable excipients and/or additives may be used in the compositions provided herein. Commonly used excipients/additives, such as pharmaceutically acceptable chelators (for example, but not limited to, EDTA, DTPA or EGTA) can optionally be added to the compositions to reduce aggregation. These additives are particularly useful if a pump or plastic container is used to administer the composition.

In some embodiments, the composition comprises a preservative. Preservatives, such as phenol, m-cresol, p-cresol, o-cresol, chlorocresol, benzyl alcohol, phenylmercuric nitrite, phenoxyethanol, formaldehyde, chlorobutanol, magnesium chloride (for example, but not limited to, hexahydrate), alkylparaben (methyl, ethyl, propyl, butyl and the like), benzalkonium chloride, benzethonium chloride, sodium dehydroacetate and thimerosal, or mixtures thereof can optionally be added to the compositions at any suitable concentration such as between about 0.001% to about 5%, or any range or value therein. The concentration of preservative used in the compositions is a concentration sufficient to yield a microbial effect. Such concentrations are dependent on the preservative selected and are readily determined by the skilled artisan.

In some embodiments, the composition is isotonic with human blood, wherein the compositions have essentially the same osmotic pressure as human blood. Such isotonic compositions will generally have an osmotic pressure from about 250 mOSm to about 350 mOSm. Isotonicity can be measured by, for example, using a vapor pressure or ice-freezing type osmometer. Tonicity of a composition is adjusted by the use of tonicity modifiers. “Tonicity modifiers” are those pharmaceutically acceptable inert substances that can be added to the composition to provide an isotonity of the composition. Tonicity modifiers suitable for the compositions provided herein include, but are not limited to, saccharides, salts and amino acids.

In certain embodiments, the composition is a pyrogen-free composition which is substantially free of endotoxins and/or related pyrogenic substances. Endotoxins include toxins that are confined inside a microorganism and are released only when the microorganisms are broken down or die. Pyrogenic substances also include fever-inducing, thermostable substances from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with proteins of interest (e.g., antibodies), even trace amounts of harmful and dangerous endotoxin must be removed. In some embodiments, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

When used for in vivo administration, the composition described herein should be sterile. The composition may be sterilized by various sterilization methods, including sterile filtration, radiation, etc. In certain embodiments, composition is filter-sterilized with a presterilized 0.22-micron filter. Sterile compositions for injection can be formulated according to conventional pharmaceutical practice as described in “Remington: The Science & Practice of Pharmacy”, 21st ed., Lippincott Williams & Wilkins, (2005). Compositions comprising proteins of interest (e.g., antibodies or CARs) such as those disclosed herein, ordinarily will be stored in lyophilized form or in solution. It is contemplated that sterile compositions comprising proteins of interest (e.g., antibody or CAR) are placed into a container having a sterile access port, for example, an intravenous solution bag or vial having an adapter that allows retrieval of the composition, such as a stopper pierceable by a hypodermic injection needle. In certain embodiments, a composition is provided as a pre-filled syringe.

In certain embodiments, the composition is a lyophilized formulation. The term “lyophilized” or “freeze-dried” includes a state of a substance that has been subjected to a drying procedure such as lyophilization, where at least 50% of moisture has been removed.

Regardless of the route of administration selected, agents provided herein, which may be used in a suitable hydrated form, and/or the pharmaceutical compositions of the provided herein, are formulated into pharmaceutically-acceptable dosage forms by conventional methods known to those of skill in the art.

### Therapeutic Methods

In certain aspects, provided herein are methods of treating a disease or disorder comprising administering to a subject an antibody or CAR described herein (e.g., a fully human antibody that has binding specificity to a peptide/MHC complex or a fully human CAR that has binding specificity to a peptide/MHC). In some embodiments, the antibody and/or CAR is an antibody and/or CAR obtained from or obtainable using the methods described herein (e.g., using a non-human animal comprising CAR loci as described herein).

In certain embodiments, provided herein are methods of treating cancer in a subject comprising administering to the subject a pharmaceutical composition described herein (e.g., a pharmaceutic composition comprising an antibody described herein, such as a fully human antibody that has binding specificity to a peptide/MHC). In some embodiments, the methods described herein can be used to treat any cancerous or pre-cancerous tumor. Cancers that may treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments, the antibody and/or CAR in the pharmaceutical composition administered to the subject has binding specificity for a peptide/MHC complex, wherein the peptide comprises an epitope of a cancer-associated antigen (e.g., an epitope expressed by the cancer being treated). Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pme117, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, MEL Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a. In some embodiments, the antigen is a neo-antigen.

In certain embodiments, provided herein are methods of treating a subject suffering from an infection, including a viral infection, a fungal infection, a bacterial infection, a helminth infection, or a protozoan infection, comprising administering to the subject a pharmaceutical composition described herein (e.g., a pharmaceutic composition comprising an antibody described herein, such as a fully human antibody that has binding specificity to a peptide/MHC). In some embodiments, the method comprises the treatment of viral infectious diseases, including HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. In some embodiments, the method comprises the treatment of parasites, such as malaria. In some embodiments, the method comprises the treatment of bacterial, fungal and other pathogenic diseases, such as Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum.

In certain embodiments, the antibody and/or CAR in the pharmaceutical composition administered to the subject has binding specificity for a peptide/MHC complex, wherein the peptide comprises an epitope of an antigen expressed by an infectious pathogen (e.g., an epitope expressed by the infectious pathogen being treated).

In some embodiments, provided herein is a method of treating an inflammatory disease, skin or organ transplantation rejection, graft-versus-host disease (GVHD), or autoimmune diseases, comprising administering to a subject a pharmaceutical composition described herein (e.g., a pharmaceutic composition comprising an antibody described herein, such as a fully human antibody that has binding specificity to a peptide/MHC). Examples of autoimmune diseases include, for example, glomerular nephritis, arthritis, dilated cardiomyopathy-like disease, ulceous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis.

In certain embodiments, the antibody and/or CAR in the pharmaceutical composition administered to the subject has binding specificity for a peptide/MHC complex, wherein the peptide comprises an epitope of a protein that is the target of an autoreactive T cell in the disease being treated (e.g., an epitope targeted by autoreactive T cells in an autoimmune disease). Exemplary proteins include targeted by autoreactive T cells include, for example, p205, insulin, thyroid-stimulating hormone, tyrosinase, TRP1, and myelin.

The pharmaceutical compositions described herein may be delivered by any suitable route of administration, including orally, nasally, as by, for example, a spray, rectally, intravaginally, parenterally, intracisternally and topically, as by powders, ointments or drops, including buccally and sublingually. In certain embodiments the pharmaceutical compositions are delivered generally (e.g., via oral or parenteral administration).

Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the antibodies and/or CARs employed in the pharmaceutical composition at levels lower than that required in order to achieve the desired therapeutic effect and gradually increase the dosage until the desired effect is achieved.

In some embodiments, the CAR receptors described here are used for T cell based therapy. For example, in certain embodiments, T cells expressing a CAR described herein are administered to a subject to induce a T cell based immune response in the subject. Methods useful in T cell based therapy is described in, for example, in Schumacher Nat. Rev. Immunol. 2:512-519 (2002) and Bitton et al., Frontiers in Bioscience 4:d386-393 (1999), each of which is incorporated by reference herein.

In some aspects, provided herein is a method of inducing an immune response (e.g., a T cell based immune response) in a subject. In some embodiments, the method includes administering to the subject a cell (e.g., a human T cell, such as a CD4 T cell or a CD8 T cell) expressing a CAR comprising a first CAR polypeptide comprising a human Ig heavy chain variable domain and a human TCRβ constant domain and a second CAR polypeptide comprising a human Ig light chain variable domain (e.g., an Ig κ variable domain or an Ig λ variable domain) and a human TCRα constant domain, wherein the CAR has binding specificity for the peptide/MHC complex. In some embodiments, the peptide/MHC complex is a peptide/class I MHC complex. In some embodiments, the peptide/MHC complex is a peptide/class II MHC complex.

In some embodiments, the subject is a subject in need thereof. In some embodiments, the subject is a subject with cancer. In such embodiments, the peptide in the peptide/MHC complex recognized by the CAR is a peptide of a cancer antigen.

In certain embodiments, provided herein are methods of treating cancer comprising administering to the subject a T cell expressing a CAR described herein. In some embodiments, the methods described herein may be used to treat any cancerous or pre-cancerous tumor. Cancers that may treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments, CAR expressed by the T cell administered to the subject has binding specificity for a peptide/MHC complex, wherein the peptide comprises an epitope of a cancer-associated antigen (e.g., an epitope expressed by the cancer being treated). Examples of cancer-associated antigens include, but are not limited to, adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pme117, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, MEL Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-lb/GAGED2a. In some embodiments, the antigen is a neo-antigen.

In some embodiments, the subject is a subject who has been infected with a pathogen. In such embodiments, the peptide in the peptide/MHC complex recognized by the CAR is a peptide of a pathogenic antigen.

Thus, in certain embodiments, provided herein are methods of treating a subject suffering from an infection, including a viral infection, a bacterial infection, a helminth infection, or a protozoan infection, comprising administering to the subject a T cell expressing a CAR described herein. For example, in some embodiments, provided herein are methods of treating viral infectious diseases, including HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. In some embodiments, the pathogen treated are parasites, such as malaria. In some embodiments, provided herein are treatments of bacterial, fungal and other pathogenic diseases, such as Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum.

In certain embodiments, the CAR expressed by the T cell administered to the subject has binding specificity for a peptide/MHC complex, wherein the peptide comprises an epitope of an antigen expressed by an infectious pathogen (e.g., an epitope expressed by the infectious pathogen being treated).

In certain aspects, provided herein is a method of inducing an immune response to a peptide/MHC complex in a subject (e.g., a human subject). In some embodiments, the method includes isolating a T cell (e.g., a CD4 T cell or a CD8 T cell) from the subject. In some embodiments, the method includes inducing expression by the T cell of a CAR that has binding specificity for the peptide/MHC complex. In some embodiments, the method includes administering the T cell to the subject. In some embodiments, the method comprises transfecting the T cell with a first vector comprising a nucleic acid sequence encoding the first CAR polypeptide and a second vector comprising a nucleic acid sequence encoding the second CAR polypeptide. In some embodiments, the method comprises transfecting the T cell with a vector comprising a nucleic sequence encoding the first CAR polypeptide and a nucleic acid sequence encoding the second CAR polypeptide. In some embodiments, the method comprises the step of inhibiting expression by the T cell of endogenous TCRα and/or TCRβ.

In some embodiments, the subject is a subject with an autoimmune disease. In such embodiments, the T cell is a regulatory T cell (i.e., a suppressor T cell) and the peptide in the peptide/MHC complex recognized by the CAR is a self-antigen to which the subject is undergoing an autoimmune response.

In some aspects, provided herein is a method of inhibiting an immune response in a subject. In some embodiments, the method includes administering to the subject a regulatory T cell (e.g., a CD4⁺, CD-25⁺ and Foxp3⁺ regulatory T cell or a Treg17 T cell) expressing a CAR comprising a first CAR polypeptide comprising a human Ig heavy chain variable domain and a human TCRβ constant domain and a second CAR polypeptide comprising a human Ig light chain variable domain (e.g., an Ig κ variable domain or an Ig λ variable domain) and a human TCRα constant domain, wherein the CAR has binding specificity for the peptide/MHC complex. In some embodiments, the peptide/MHC complex is a peptide/class I MHC complex. In some embodiments, the peptide/MHC complex is a peptide/class II MHC complex.

In certain aspects, provided herein is a method of inhibiting an immune response to a peptide/MHC complex in a subject (e.g., a human subject). In some embodiments, the method includes isolating a regulatory T cell (e.g., a CD4⁺, CD-25⁺ and Foxp3⁺ regulatory T cell or a Treg17 T cell) from the subject. In some embodiments, the method includes inducing expression by the T cell of a CAR that has binding specificity for the peptide/MHC complex. In some embodiments, the method includes administering the T cell to the subject. In some embodiments, the method comprises transfecting the T cell with a first vector comprising a nucleic acid sequence encoding the first CAR polypeptide and a second vector comprising a nucleic acid sequence encoding the second CAR polypeptide. In some embodiments, the method comprises transfecting the T cell with a vector comprising a nucleic sequence encoding the first CAR polypeptide and a nucleic acid sequence encoding the second CAR polypeptide. In some embodiments, the method comprises the step of inhibiting expression by the T cell of endogenous TCRα and/or TCRβ.

In some embodiments, the subject is a subject with an autoimmune disease. In such embodiments, the T cell is a regulatory T cell (i.e., a suppressor T cell) and the peptide in the peptide/MHC complex recognized by the CAR is a self-antigen to which the subject is undergoing an autoimmune response.

Thus, in some embodiments, the methods described herein may be used to treat diseases or disorders related to a deleterious immune response, such as asthma, inflammatory disease, skin or organ transplantation, graft-versus-host disease (GVHD), or autoimmune diseases. Examples of autoimmune diseases include, for example, glomerular nephritis, arthritis, dilated cardiomyopathy-like disease, ulceous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis.

In certain embodiments, CAR expressed by the T cell administered to the subject has binding specificity for a peptide/MHC complex, wherein the peptide comprises an epitope of a protein that is the target of an autoreactive T cell in the disease being treated (e.g., an epitope targeted by autoreactive T cells in an autoimmune disease). Exemplary proteins include targeted by autoreactive T cells include, for example, p205, insulin, thyroid-stimulating hormone, tyrosinase, TRP1, and myelin.

### Nucleic Acid Molecules

Provided herein are nucleic acid molecules that encode the antibodies, CARs and portions of antibodies and CARs described herein. In some embodiments, the nucleic acid encodes a variable domain (e.g., a heavy and/or light chain variable domain) of an antibody or CAR described herein. The nucleic acids may be present, for example, in whole cells, in a cell lysate, or in a partially purified or substantially pure form.

In certain aspects, provided herein are nucleic acids encoding an antibody and/or CAR polypeptide described herein or a portion thereof. The nucleic acids may be present, for example, in whole cells, in a cell lysate, or in a partially purified or substantially pure form. Nucleic acids described herein can be obtained using standard molecular biology techniques. For example, nucleic acid molecules described herein can be cloned using standard PCR techniques or chemically synthesized. For nucleic acids encoding CARs or antibodies expressed by hybridomas, cDNAs encoding each chain of the antibody or CAR made by the hybridoma can be obtained by standard PCR amplification or cDNA cloning techniques.

In certain aspects, provided herein is a nucleic acid composition comprising a first nucleic acid sequence encoding a first CAR polypeptide comprising an Ig heavy chain variable domain and a TCRβ constant domain and a second nucleic acid sequence encoding a second CAR polypeptide comprising an Ig light chain variable domain (e.g., an Ig κ variable domain or an Ig λ variable domain) and a TCRα constant domain, wherein a CAR comprising the first CAR polypeptide and the second CAR polypeptide has binding specificity for a peptide/MHC complex. In certain embodiments, the Ig heavy chain variable domain and/or the Ig light chain variable domain are human Ig variable domains. In some embodiments, the TCRβ constant domain and/or the TCRα constant domain are rodent constant domains (e.g., rat constant domains or mouse constant domains). In some embodiments, the TCRβ constant domain and/or the TCRα constant domain are human constant domains. In some embodiments, the first nucleic acid sequence and the second nucleic acid sequence are on a single nucleic acid molecule. In some embodiments, the first nucleic acid sequence and the second nucleic acid sequence are on separate nucleic acid molecules.

In certain aspects, provided herein is a nucleic acid composition comprising a first nucleic acid sequence encoding the heavy chain of an antibody described herein and a second nucleic acid sequence encoding a light chain of an antibody described herein (e.g., an Ig κ light chain or an Ig λ light chain), wherein a an antibody comprising the heavy chain and the light chain has binding specificity for a peptide/MHC complex. In certain embodiments, the Ig heavy chain variable domain and/or the Ig light chain variable domain are human Ig variable domains. In some embodiments, the Ig heavy chain constant domain and/or the Ig light chain constant domain are rodent constant domains (e.g., rat constant domains or mouse constant domains). In some embodiments, the Ig heavy chain constant domain and/or the Ig light chain constant domain are human constant domains. In some embodiments, the first nucleic acid sequence and the second nucleic acid sequence are on a single nucleic acid molecule. In some embodiments, the first nucleic acid sequence and the second nucleic acid sequence are on separate nucleic acid molecules.

In certain embodiments, provided herein are vectors that contain the nucleic acid molecules described herein. As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”).

In certain embodiments, provided herein are cells that contain a nucleic acid described herein (e.g., a nucleic acid encoding an antibody or CAR described herein or a portion thereof). The cell can be, for example, prokaryotic, eukaryotic, mammalian, avian, murine and/or human. In certain embodiments the nucleic acid described herein is operably linked to a transcription control element such as a promoter. In some embodiments the cell transcribes the nucleic acid described herein and thereby expresses an antibody, antigen binding fragment thereof or polypeptide described herein. The nucleic acid molecule can be integrated into the genome of the cell or it can be extrachromasomal.

Nucleic acid molecules provided herein can be obtained using standard molecular biology techniques. For example, nucleic acid molecules described herein can be cloned using standard PCR techniques or chemically synthesized.

Once DNA fragments encoding a V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the heavy chain variable region can be converted to a full-length heavy chain gene by operatively linking the heavy chain variable region DNA to another DNA molecule encoding heavy chain constant regions (e.g., CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain CH1 constant region.

The isolated DNA encoding the light chain variable region can be converted to a full-length light chain gene (as well as a Fab light chain gene) by operatively linking the light chain variable region encoding DNA to another DNA molecule encoding a light chain constant region. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

### EXAMPLES

### Example 1

### Activation of T Cells Bearing Chimeric Antigen Receptors

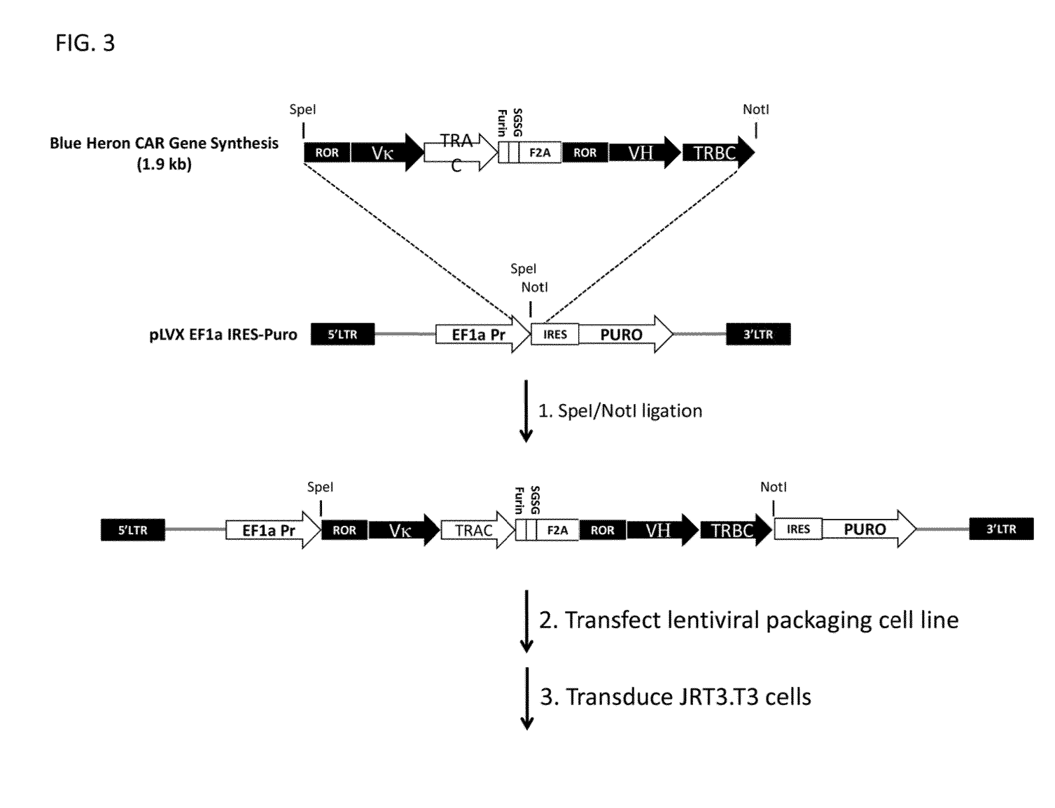

Sequences encoding VH and VL domains of antibodies recognizing peptide-MHC complexes (NY-ESO-1 and MAGE-1 peptides, see Stewart-Jones et al. (2009) Rational development of high-affinity T-cell receptor-like antibodies, Proc. Nat'l. Acad. Sci. USA 106:5784-88 and Hülsmeyer et al. (2005) A Major Histocompatibility Complex Peptide-restricted Antibody and T Cell Receptor Molecules Recognize Their Target by Distinct Binding Modes, J. Biol. Chem. 280:2972-80, respectively, incorporated herein by reference) were incorporated into synthetically produced 1.9 kb nucleotide sequences, where the immunoglobulin V_{κ} and V_{H} domain sequences were placed upstream of TCRA C and TCRB C sequences, respectively (FIG. 3). Stewart-Jones et al. describe an antibody recognizing NY-ESO-1 peptide (SLLMWITQVNY, SEQ ID NO: 1) complexed with HLA-A2; Hülsmeyer et al. describe an antibody recognizing MAGE-1 peptide (EADPTGHSY, SEQ ID NO: 2) complexed with HLA-A1.

Synthesized sequence comprising anti-NY-ESO-1/A2 and anti-MAGE-1/A1 VHs and VLs also comprised ROR leader sites (U.S. Pat. No. 7,534,604, incorporated herein by reference) upstream of both V_{κ} and V_{H}, with a furin cleavage site and self-cleaving F2A peptide for bicistronic expression (Yang et al. (2008) Development of optimal bicistronic lentiviral vectors facilitates high-level TCR gene expression and tumor cell recognition, Gene Ther. 15:1411-1423, incorporated by reference). Synthesized DNA was obtained from Blue Heron. As a negative control, germline Vκ1-39Jκ5 [ULC1-39] or Vκ3-20Jκ1 [ULC3-20] and V_{H}3-23J_{H}4 [UHC] were incorporated into lentiviral vectors upstream of TCRA C and TCRA B, respectively (see US Patent Application Publication Nos. US 2011/0195454 for ULC1-39 and ULC3-20 and US 2014/0245468 for UHC, both incorporated herein by reference).

Each synthesized 1.9 kb DNA sequence (i.e., sequence encoding MAGE-1 CAR, NY-ESO-1 CAR, ULC1-39UHC and ULC3-20UHC CAR) was ligated into a multiple cloning site of pLVX EF1a IRES-PURO lentiviral vector (Clontech). Constructs were packaged with delta 8.9 and PMDG via transient transfection of 293T cells, and viral supernatant was produced and subsequently used to transduce J.RT3-T3.5 (T3) cells, which are derived from Jurkat T cells but lack the ability to produce TCRαβ heterodimers or express CD3 on their plasma membrane. Jurkat (CD4+CD8− T) cells were used as a control. After transduction, T3 cells were screened via FACS sorting for their ability to express cell-surface CD3, and all CAR transduced T3 cells exhibited expression of CD3, indicating that CAR molecules were expressed on cell surface.

T cell activation of T3 cells transduced with either CARs comprising VL and VH of NY-ESO-1 or VL and VH of MAGE-1 was determined by ELISPOT assay (Czerkinsky et al. (1983) A solid-phase enzyme-linked immunospot (ELISPOT) assay for enumeration of specific antibody-secreting cells, J. Immunol. Methods 65:109-121, and Miyahira et al. (1995) Quantification of antigen specific CD8+ T cells using an ELISPOT assay. J. Immunol. Methods, 181: 45-54, incorporated by reference) that detected secretion of human IL-2. The human IL-2 ELISPOT kit was obtained from BD Biosciences (Catalog #551282)

Specifically, T3 cells, either alone or expressing MAGE-1 CAR (A1MAGECAR), NY-ESO-1 CAR (A1NYESOCAR) or ULC1-39UHC CAR, were added to wells of an ELISPOT plate at a concentration of 5×10⁵ cells/well. Target K562 cells (a MHC free cell line), either alone or expressing human HLA-A1 (K562_A1) or human HLA-A2 (K562_A2), were added at concentration of 1×10⁵ cells/well (5:1 ratio of effectors to target cells). Appropriate peptides (MAGE-1, HLA-A1 restricted or NY-ESO-1, HLA-A2 restricted) were added to the wells at a concentration of 10 μg/mL, the plate was incubated at 37° C. for 16-20 hours, and developed according to manufacturer's instructions.

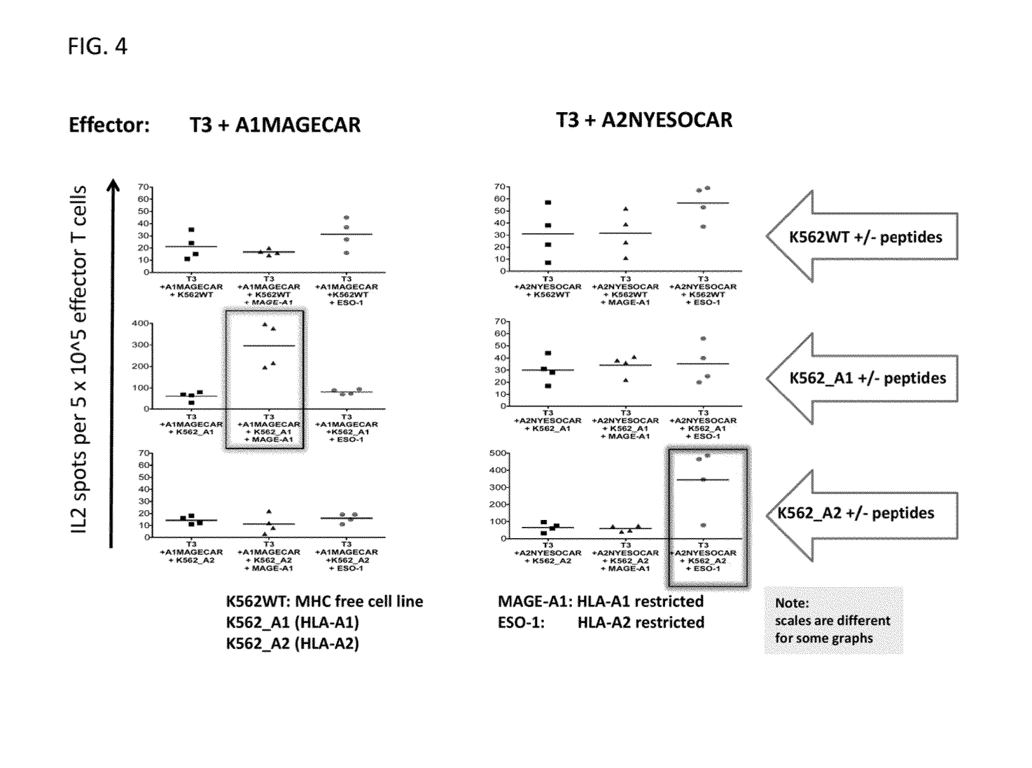

Activation of T cells was assessed based on the number of cytokine-secreting cells detected by the black-blue colored precipitate that forms at the site of cytokine localization. As shown in FIG. 4, activation only occurred in cells expressing A1MAGECAR that were treated with MAGE-1 HLA-A1 restricted peptide (MAGE-A1) in the presence of effector cells expressing HLA-A1 (K562_A1), and did not occur in effector cells expressing HLA-A2 (K562_A2) or wild type K562 cells (K562WT). Moreover, T3 cells expressing A1MAGECAR were not activated by K562 cells expressing HLA-A1 in the presence of an unrelated peptide. Similarly, only T3 cells expressing A2NYESOCAR that were treated with NY-ESO-1 HLA-A2 restricted peptide (ESO-1) in the presence of effector cells expressing HLA-A2 (K562_A2) were activated, as evident by IL-2 secretion. T3 cells expressing ULC1-39UHC CAR (negative control) were not activated by of any peptide-MHC complex.

### Example 2

### Generation of a Genetically Modified Mouse Expressing a Chimeric Antigen Receptor

### Example 2.1

### Construction of Chimeric Human Igκ Variable-Mouse TCRA Constant Locus

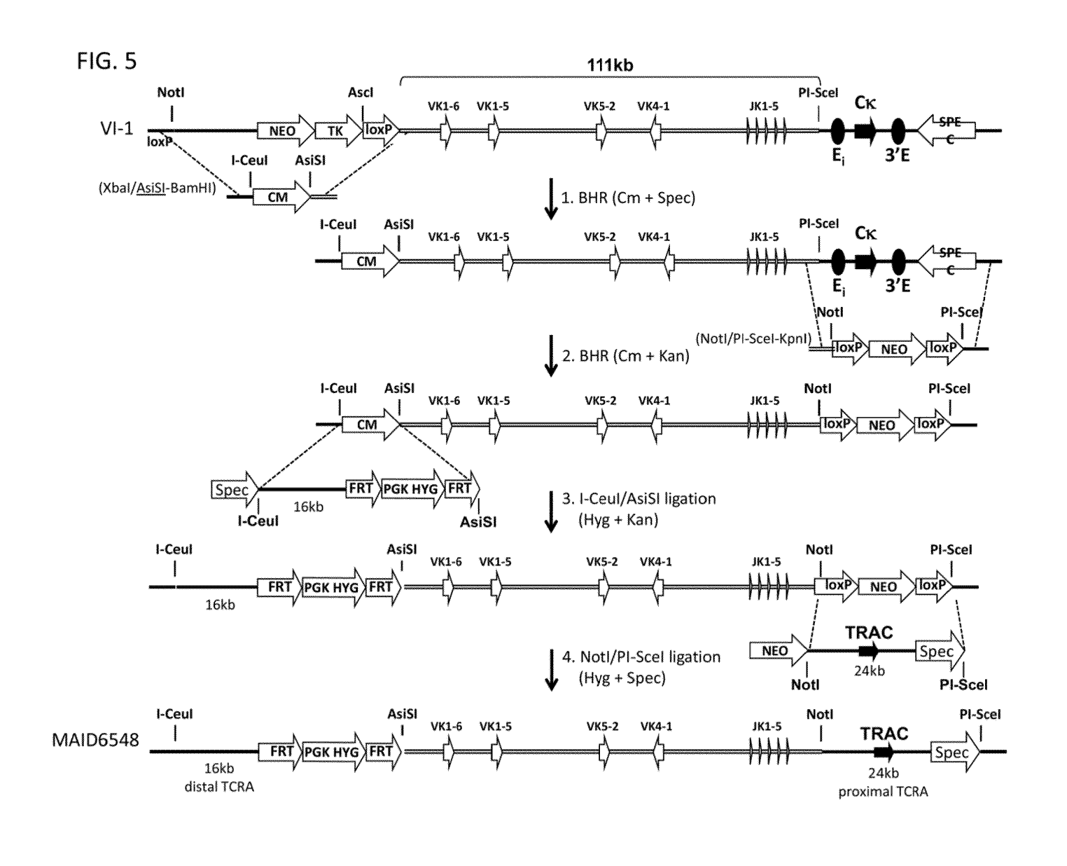

A VELOCIMMUNE® Igκ large targeting vector (LTVEC) containing all human J_{κ} segments and 4 functional human V_{κ} segments (“VI-1”, see Macdonald et al. (2014) Precise and in situ genetic humanization of 6 mB of mouse immunoglobulin genes, Proc. Natl Acad. Sci USA 111:5147-52 and Supplemental Information) was modified by Bacterial Homologous Recombination (BHR) to replace the 5′ mouse κ arm and neo-tk-loxp cassette with a chloramphenicol (CM)-resistance cassette flanked by unique I-CeuI and AsiSI sites (FIG. 5, step 1.). In step 2, the construct generated in step 1 was further modified by BHR to replace the 3′ mouse κ arm and Spec cassette with a loxp-neo-loxp cassette flanked by unique NotI and PI-SceI sites. Subsequently (step 3), an I-CeuI-AsiSI nucleic acid fragment containing a 16 kb distal mouse Tcra arm and an Frt-Hyg-Frt cassette was ligated into the construct of step 2 to replace the CM cassette. Finally (step 4), a NotI-PI-SceI nucleic acid fragment containing a 24 kb proximal mouse Tcra arm and a Spec cassette, was ligated into the NotI and PI-SceI sites of the construct of step 3 replacing the loxp-neo-loxp cassette, creating the final LTVEC, designated MAID 6548.

The final LTVEC contained from 5′ to 3′: (1) a 16 kb 5′ mouse Tcra arm for homologous recombination in ES cells (genome location 14:52411629-52427793, all coordinates based on mouse assembly GRCm38), (2) a Frt-Hyg-Frt cassette for selection in E. coli or ES cells, (3) 111 kb of human κ locus DNA containing the 4 most proximal V_{κ} segments and all 5 J_{κ} segments (J1-J5), (4) a 24 kb 3′ mouse TCRA arm for homologous recombination in ES cells, which includes the TCRA constant gene (genome location 14:54218920-54243117), and (5) a Spec cassette for selection in E. coli.

The LTVEC (MAID6548) has the following junction sequences, where mouse sequences are in parentheses, human sequences are in normal font, multiple cloning sites are bolded, and Frt sequences are italicized (Table 1).

TABLE 1
Junction Sequences of Igκ V-Tcra C Large Targeting
Vector (5′ to 3′)

| | Seq ID | |
|---|---|---|
| Junction | No | Sequence |
| mouse Tcra/ | 3 | (ATGGAGTAGTCAGAACACACTCTTCA |
| Frt | | GAAGGGACTCCTGATTTCAAAGGG)GG |
| | | GTACCGGGCCCCCCCTCGAGAAGTTCC |
| | | TATTCCGAAGTTCCTATTCTC |
| Frt/human | 4 | TCCGAAGTTCCTATTCTCTAGAAAGTATA |
| Igκ | | GGAACTTCCTAGGGCGATCGCGTGCATG |
| | | GCACTGACATAGGCCATTGTTAACAGGG |
| | | TCCCAGCAGCTGGTC |
| Human IgK/ | 5 | GATAAATTATTTTGTCAGACAACAATAA |
| mouse Tcra | | AAATCAATAGCACGCCCTAAGAGCGGCC |
| | | GCCACCGCGGTGGAGCTC(AGGTTTCCG |
| | | GTACTTAACAACAGAGCACAGATTTAGT |
| | | GGTGAGGGACTCT) |

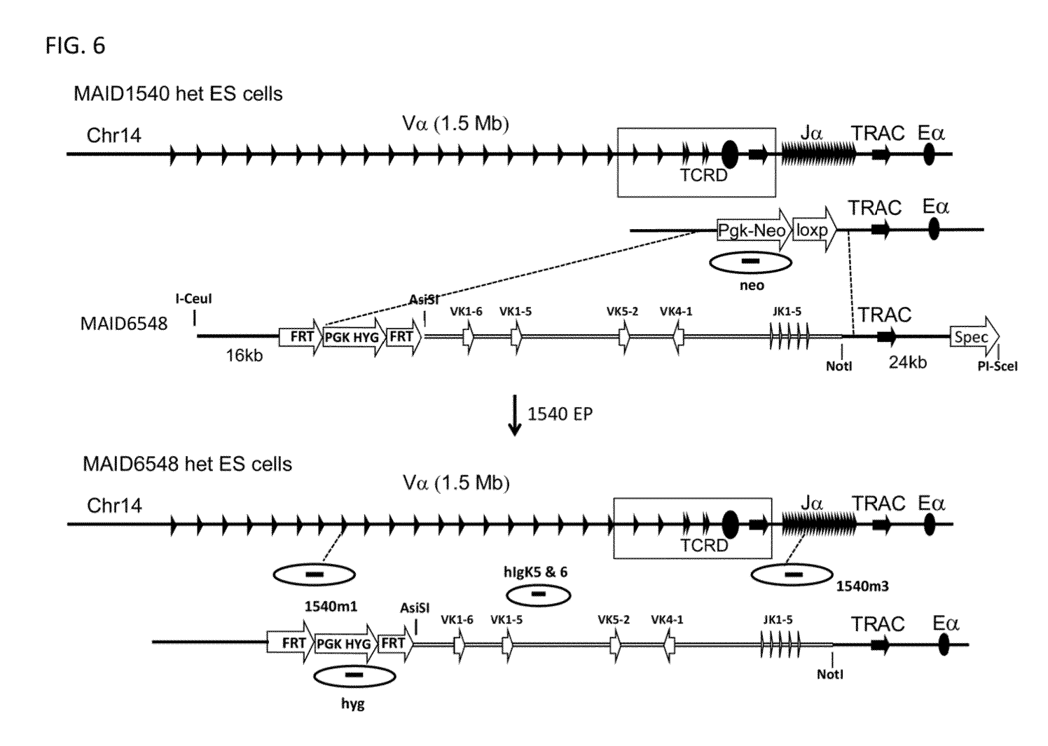

MAID6548 was used to electroporate into MAID1540 het ES cells (see FIG. 4A of U.S. Pat. No. 9,113,616, incorporated herein by reference), in which all of the mouse TCRA V and J segments have been deleted and replaced with a Neo cassette (FIG. 6). The junction sequence of the resulting locus are the same as described in Table 1 above. Hyg-resistant ES cells were screened using TAQMAN® assays to identify correctly targeted clones (see, e.g., Lie and Petropoulos, 1998. Curr. Opin. Biotechnology 9:43-48, incorporated herein by reference) (Table 2; LOA=loss of allele; GOA=gain of allele, regions recognized are indicated in FIG. 6).

TABLE 2
TAQMAN Primers and Probes

| Gene | Assay | Fwd Primer | Probe | Rev Primer |
|---|---|---|---|---|
| Neo | LOA | GGTGGAGAGGCT | TGGGCACAACAG | GAACACGGC |
| | | ATTCGGC (SEQ | ACAATCGGCTG | GGCATCAG |
| | | ID NO: 6) | (SEQ ID NO: 7) | (SEQ ID NO: 8) |
| Hyg | GOA | TGCGGCCGATCT | ACGAGCGGGTTC | TTGACCGAT |
| | | TAGCC (SEQ ID | GGCCCATTC | TCCTTGCGG |
| | | NO: 9) | (SEQ ID NO: 10) | (SEQ ID NO: 11) |
| hIgK6 | GOA | GTCAAGCACTGCT | AACCCTTGTGCT | TGTTGTAGA |
| | | GGCACAC (SEQ ID | ATTGAATTGCTATGCTGTCAG | CCCTCCGCCAC |
| | | NO: 12) | (SEQ ID NO: 13) | (SEQ ID NO: 14) |
| hIgK5 | GOA | CCCCGTCCTCCTC | TCATGTCCATTA | TGCAAGTGCT |
| | | CTTTTTC (SEQ ID | ACCCATTTACCTTTTGCCCA | GCCAGCAAG |
| | | NO: 15) | (SEQ ID NO: 16) | (SEQ ID NO: 17) |
| Parental | | CAGTAAGGGAAG | TGCACACTGCTC | TGCTGGTGGC |
| 1540 m1 | | AGACTACAACAGC | ACCACTGCAAGCTAT | CCCATCT |
| | | AT (SEQ ID NO: 18) | (SEQ ID NO: 19) | (SEQ ID NO: 20) |
| Parental | | GAACTCAGCTAT | CAGCCCAGCAG | GCTCAGGGAG |
| 1540 m3 | | GATAGTGTCGAATG | CTGTGGGTTCTC | AACACAGAACTTAG |
| | | TA (SEQ ID NO: 21) | (SEQ ID NO: 22) | A (SEQ ID NO: 23) |

If desired, additional human V_{κ} segments can be added to the TCR variable region locus using LTVECs having the same 16 kb 5′ mouse Tcra homology arm described above linked to human Igκ sequences that overlap with the initial insertion.

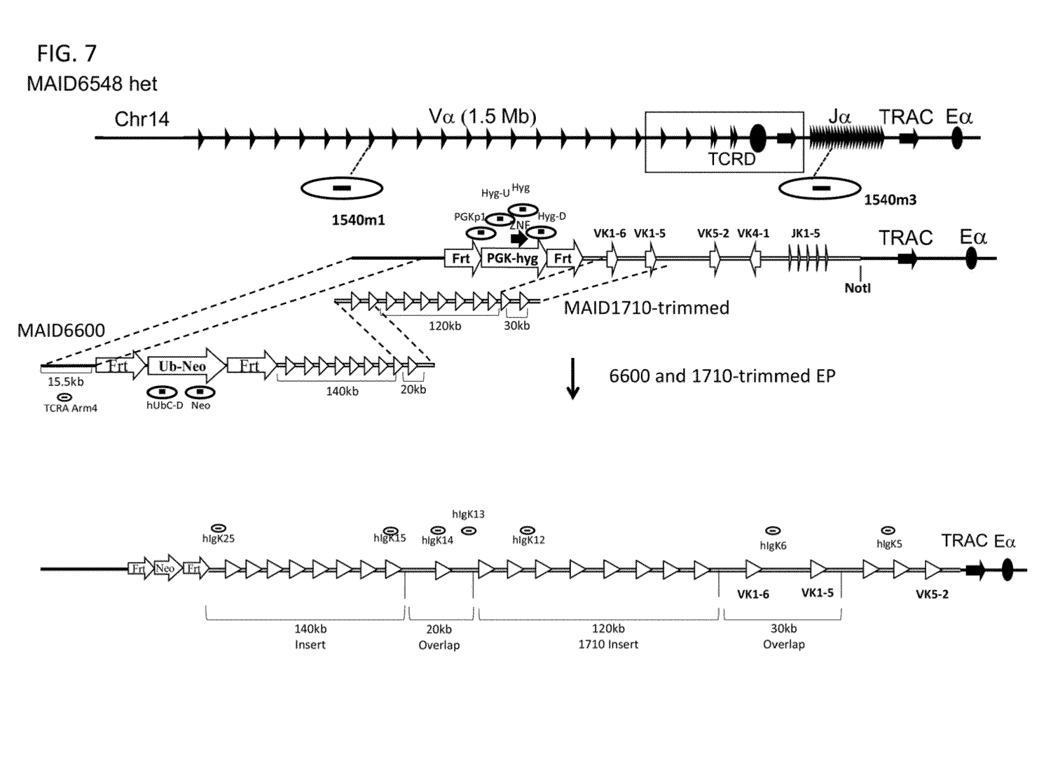

Different strategies can by utilized to generate such ES cells. One approach, summarized in FIG. 7, involves double targeting or co-electroporation of two different large targeting vectors into ES cells. In this approach, the first large targeting vector (labeled as MAID 1710, derived from a restriction digest of a vector constructed as described in U.S. Patent Application Publication No. 2012/0096512A1, incorporated herein by reference) comprises a 3′ 30 kb homology arm that includes the sequence of human Vκ1-5 and Vκ1-6 gene segments, a 120 kb sequence that comprises human Vκ3-7 to Vκ3-15 gene segments, and a 5′ 20 kb region (“overlap region”) that comprises human Vκ1-16 gene segment. The second large targeting vector (labeled as MAID 6600, also derived from a vector constructed as described in U.S. Patent Application Publication No. 2012/0096512A1) comprises a 3′ 20 kb overlap region (region comprising human Vκ1-16 gene segment, same as in the first vector), a 140 kb sequence comprising human Vκ1-17 to Vκ2-30 gene segments, a FRT-Ub-Neo-FRT selection cassette and a 15.5 kb 3′ mouse TCR A homology arm. The ES cells generated in FIG. 6 (MAID 6548, heterozygous for all human Jκ segments and four functional human Vκ gene segments) were electroporated with the two large targeting vectors described above along with a nucleic acid encoding a modified Zinc Finger Nuclease (ZFN) that targets the hygromycin gene at nucleotide sequence TGCGATCGCTGCGGCCGAtcttagCCAGACGAGCGGGTTCGG (with cleavage site in lower case letters; SEQ ID NO:24) and promotes double stranded breaks at the Hyg sequence. The two co-electroporated large targeting vectors were inserted by homologous recombination into the DNA sequence, replacing the region containing and surrounding the Hyg selection cassette. The resulting ES cells contained at the endogenous TCRα locus a human immunoglobulin variable region comprising human Jκ1 to Jκ5 and Vκ4-1 to Vκ2-30 gene segments. Successful incorporation of the two large targeting vectors was confirmed using the TAQMAN® assays described above (Lie and Petropoulos, supra), using probes and primers indicated in FIG. 7 and listed in Table 3 below (GOA=gain of allele; LOA=loss of allele; copy number=check for copy number of sequence to trace transgenic integration vs. targeted integration; hArm1=30 kb 3′ homology arm of the first large targeting vector (MAID 1710); hArm2=20 kb overlap of the first (MAID 1710) and the second (MAID 6600) large targeting vectors, mArm=15.5 kb 5′ homology arm of the second targeting vector (MAID 6600), WT mouse control-sequences present at the mouse TCRα locus).

TABLE 3
TAQMAN Primers and Probes

| Gene | Assay | Fwd Primer | Probe | Rev Primer |
|---|---|---|---|---|
| HYG | LOA | TGCGGCCGATC | ACGAGCGGGTTCG | TTGACCGATTC |
| | | TTAGCC (SEQ ID | GCCCATTC (SEQ ID | CTTGCGG (SEQ |
| | | NO: 25) | NO: 26) | ID NO: 27) |
| HYG-U | LOA | CGACGTCTGTC | AGTTCGACAGCGTG | CACGCCCTCCTA |
| | | GAGAAGTTTCTG | TCCGACCTGA (SEQ | CATCGAA (SEQ |
| | | (SEQ ID NO: 28) | ID NO: 29) | ID NO: 30) |
| Hyg-D | LOA | TGTCGGGCGTAC | CCGTCTGGACCGAT | GGGCGTCGGTTT |
| | | ACAAATCG (SEQ | GGCTGTGT (SEQ ID | CCACTATC (SEQ |
| | | ID NO: 31) | NO: 32) | ID NO: 33) |
| PGKp1 (Hyg | LOA | CAAATGGAAGT | CTCGTGCAGATGGA | CCGCTGCCCCAA |
| Promoter) | | AGCACGTCTCAC | CAGCACCGC (SEQ | AGG (SEQ ID |
| | | T (SEQ ID NO: 34) | ID NO: 35) | NO: 36) |
| hIgK6 | Copy number | GTCAAGCACTGC | AACCCTTGTGCTAT | TGTTGTAGACCC |
| | of hArm1 | TGGCACAC (SEQ | TGAATTGCTATGCT | TCCGCCAC (SEQ |
| | | ID NO: 37) | GTCAG (SEQ ID | ID NO: 39) |
| | | | NO: 38) | |
| hIgK12 (MAID | GOA | TTGCCTTTCTCA | CAGCCCATCCTGTC | TGGCCCAACAGT |
| 1710 insert) | | CACCTGCAG | ACTTCGCTGGA | ACAGCTCAG |
| | | (SEQ ID NO: 40) | (SEQ ID NO: 41) | (SEQ ID NO: 42) |
| hIgK13 | Copy number | TCAGTCAATCAC | TCCCCAGGTAGCCT | CACATTACTGAG |
| | of hArm2 | CTTTCCCAGC | CATGAACCAATGTT | TCCCCACAGGG |
| | | (SEQ ID NO: 43) | (SEQ ID NO: 44) | (SEQ ID NO: 45) |
| hIgK14 | Copy number | CATTGTCAAAGA | ACCATTGCAGTTTA | TCTTGCAATGGG |
| | of hArm2 | AGCACTGGAAATG | CCCACGGTTAGGAT | ATCATCAGATG |
| | | (SEQ ID NO: 95) | TTTT (SEQ ID NO: 46) | (SEQ ID NO: 47) |
| Neo | GOA | GGTGGAGAGGC | TGGGCACAACAGA | GAACACGGCGG |
| | | TATTCGGC (SEQ | CAATCGGCTG (SEQ | CATCAG (SEQ ID |
| | | ID NO: 48) | ID NO: 49) | NO: 50) |
| hIgK15 | GOA | CAGGTGCAAAG | TGGGTCCTGCCCAT | GGCAGCCTGAGT |
| | | GTGACCACAG | CCATGCA (SEQ ID | GTCAGAGC (SEQ |
| | | (SEQ ID NO: 101) | NO: 102) | ID NO: 103) |
| hIgK25 | GOA | GTTCAGGCCCCA | TCCTCTCTGGAGCA | CCTGAAGCCATG |
| | | CAGACTCTC | ACCATGAAGTTCCC | AGGGCAG (SEQ |
| | | (SEQ ID NO: 51) | T (SEQ ID NO: 52) | ID NO: 53) |
| hUbC-D (Neo | GOA | AGGGTAGGCTCT | ACAGGCGCCGGAC | CCAAAGAAACT |
| Promoter) | | CCTGAATCG | CTCTGGT (SEQ ID | GACGCCTCAC |
| | | (SEQ ID NO: 54) | NO: 55) | (SEQ ID NO: 56) |
| TCRA Arm4 | Copy number | GCGCCACATGAA | TGTACCCAATCTTC | GGCATCCTGTCC |
| | of mArm | TTTGACCAG | CAAAGAAAGAGCT | TCCCTTC (SEQ ID |
| | | (SEQ ID NO: 57) | G (SEQ ID NO: 58) | NO: 59) |
| Parental | WT mouse | CAGTAAGGGAA | TGCACACTGCTCAC | TGCTGGTGGCCC |
| 1540m1 | control | GAGACTACAAC | CACTGCAAGCTAT | CATCT (SEQ ID |
| | | AGCAT (SEQ ID | (SEQ ID NO: 61) | NO: 62) |
| | | NO: 60) | | |
| Parental | WT mouse | GAACTCAGCTAT | CAGCCCAGCAGCTG | GCTCAGGGAGA |
| 1540m3 | control | GATAGTGTCGAA | TGGGTTCTC (SEQ | ACACAGAACTTA |
| | | TGTA (SEQ ID | ID NO: 64) | GA (SEQ ID |
| | | NO: 63) | | NO: 65) |
| hIgK5 | MAID 6548 | CCCCGTCCTCCT | TCATGTCCATTAAC | TGCAAGTGCTGC |
| | sequence | CCTTTTTC (SEQ | CCATTTACCTTTTG | CAGCAAG (SEQ |
| | (see | ID NO: 66) | CCCA (SEQ ID | ID NO: 68) |
| | Table 2) | | NO: 67) | |

The resulting targeted locus in ES cells has the following junction sequences, where mouse sequences are in parentheses, human sequences are in normal font, multiple cloning sites are bolded, and Frt sequences are italicized (Table 4).

TABLE 4
Junction Sequences of Locus Resulting from Double
ES Cell Targeting (5′ to 3′)

| | Seq ID | |
|---|---|---|
| Junction | No | Sequence |
| mouse Tcra/ | 69 | (GTCTTTTTTGTTCTTCACAGTTGAGCTTCA |
| 5′ Frt | | TCAAAGTCACATGGGTTAAACTCTATGGAG |
| | | TAGTCAGAACACACTCTTCA)GAAGGGACTC |
| | | CTGATTTCAAAGGGTACCGAAGTTCCTATT |
| | | CCGAAGTTCCTATTCTCTAGAAAGTATAGGA |
| | | ACTTC |
| 3′ Frt/ | 70 | GAAGTTCCTATTCCGAAGTTCCTATTCTC |
| human IgK | | TAGAAAGTATAGGAACTTCCTAGGGTTT |
| | | CACCGGTGGCGCGCCTAACAGAGAGGAAAGT |
| | | CAAATTATAAAGAATATGAGATTCAGAATTC |
| | | TGATTAACTGTGG |
| Human IgK/ | | Same as in Table 1 |
| mouse Tcra | | |

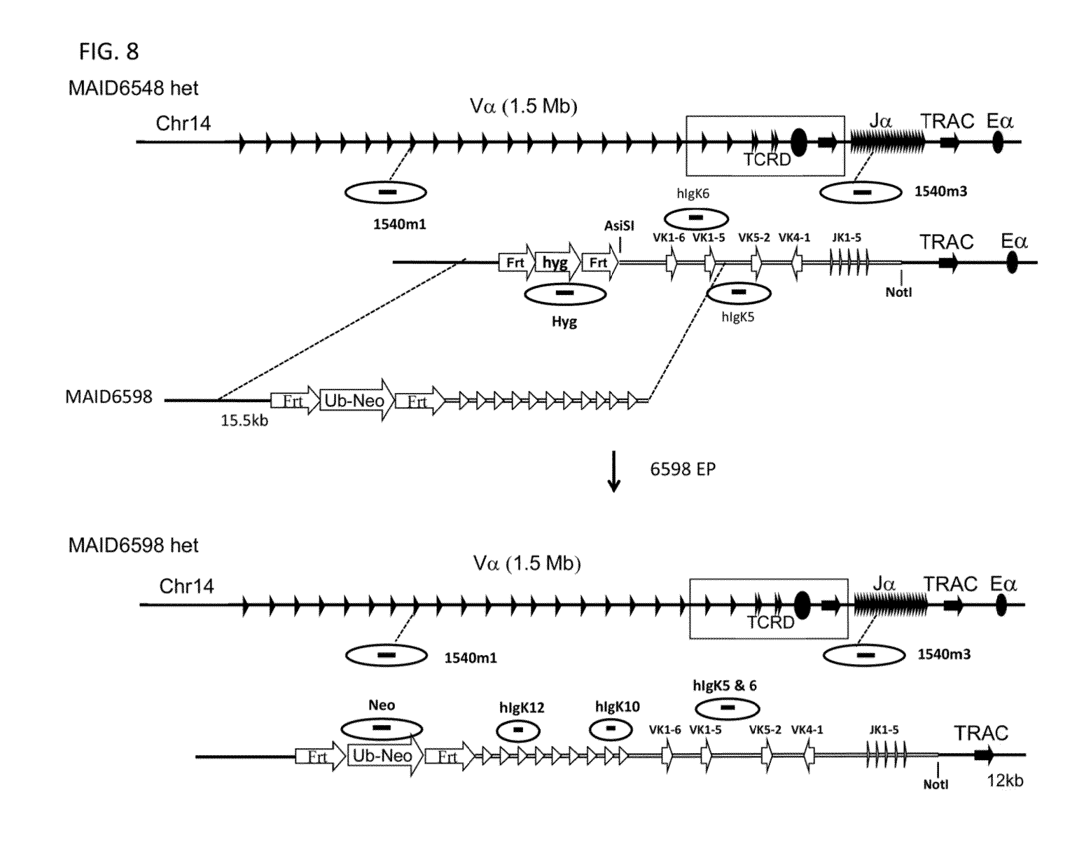

An alternative strategy for generating TCRα loci comprising additional immunoglobulin variable region gene segments involves serial targeting with large targeting vectors comprising additional variable gene segments (see, e.g., FIG. 8). ES cells heterozygous for all human Jκ gene segments and four functional human Vκ gene segments (MAID 6548; see FIG. 6) are electroporated with a large targeting vector comprising, from 5′ to 3′: a 15.5 kb 5′ mouse homology arm, an Frt-Ub-Neo-Frt selection cassette, an 120 kb fragment comprising Vκ3-7 to Vκ3-15 gene segments, and a 30 kb 3′ human homology arm comprising Vκ1-5 and Vκ1-6 gene segments (also present in MAID 6548 sequence). Successful incorporation is confirmed with Taqman assay described above, using primers and probes that are listed in Table 3 above and indicated in FIG. 8: Hyg, hIgKS, hIgK6, hIgK12, Neo, parental 1540m3, parental 1540m1. An additional set of primers and probe, hIgK10, is also used to confirm successful incorporation: Fwd Primer—CGATTATGACTGGTTAGGTAGAAAGGTG (SEQ ID NO:71); Probe—GCCACTGGTTTCTCCAAATGTTTTCAATCCAT (SEQ ID NO:72); Rev. Primer—GGGAGTACTTGGAGATCCCTAAGC (SEQ ID NO:73).

The resulting targeted locus in ES cells has the following junction sequences, where mouse sequences are in parentheses, human sequences are in normal font, multiple cloning sites are bolded, and Frt sequences are italicized (Table 5).

TABLE 5
Junction Sequence of Locus Resulting from Single
ES Cell Targeting (5′ to 3′)

| | Seq ID | |
|---|---|---|
| Junction | No | Sequence |
| mouse Tcra/ | 74 | (TTGAGCTTCATCAAAGTCACATGGGTTAAA |
| 5′ Frt | | CTCTATGGAGTAGTCAGAACACACTCTTCA) |
| | | GAAGGGACTCCTGATTTCAAAGGGTACCGA |
| | | AGTTCCTATTCCGAAGTTCCTATTCTCTAG |
| | | AAAGTATAGGAACTTC |
| 3′ Frt/ | 75 | GAAGTTCCTATTCCGAAGTTCCTATTCTCTA |
| human IgK | | GAAAGTATAGGAACTTCCTAGGGTTTCACCG |
| | | GTGGCGCGCCAGGACCCAGGCTCTGACACT |
| | | CAGGCTGCCAATACAATTGCCATGAAGACA |
| | | GATGTTGATG |
| Human IgK/ | | Same as in Table 1 |
| mouse Tcra | | |

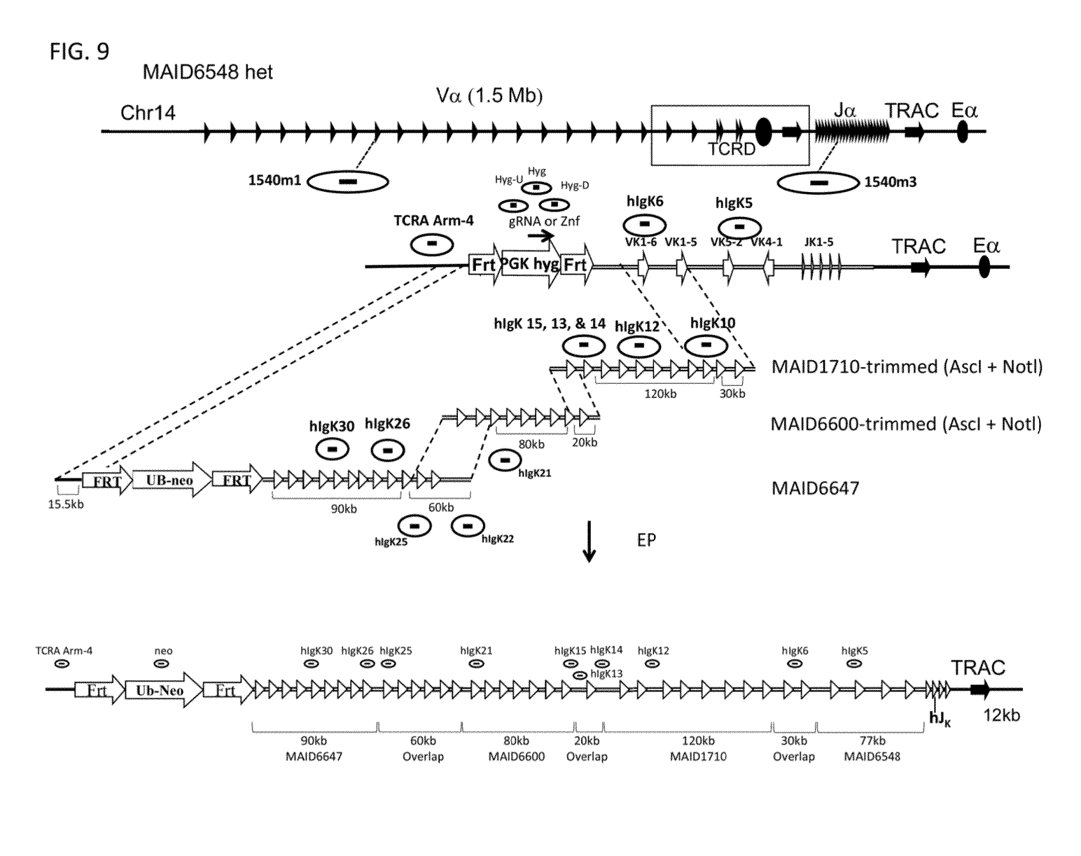

Upon completion of the single targeting depicted in FIG. 8 or the double targeting depicted in FIG. 7, the ES cells may be successively targeted with large targeting vectors comprising additional Vκ gene segments in order to incorporate a complete repertoire of functional human immunoglobulin Vκ gene segments, e.g., all functional human Vκ gene segments in the proximal V cluster. As an alternative to either the double or the successive single targeting methods depicted in FIGS. 8 and 7, a triple targeting method can be used to generate ES cells comprising up to the entire repertoire of functional human immunoglobulin Vκ gene segments, e.g., all functional human Vκ gene segments in the proximal V cluster. In this approach, which is depicted in FIG. 9, the first large targeting vector (MAID 1710, trimmed with AscI and NotI restriction enzymes, see above) comprises a 3′ 30 kb homology arm that includes human Vκ1-5 and Vκ1-6 gene segment sequences, a 120 kb sequence that comprises human Vκ3-7 to Vκ3-15 gene segment sequences, and a 5′ 20 kb region (“overlap region”) that comprises human Vκ1-16 gene segment. The second large targeting vector (MAID 6600, trimmed with AscI and NotI restriction enzymes, see above) comprises a 3′ 20 kb overlap region (region comprising human Vκ1-16 gene segment, same as in the first vector), an 80 kb sequence comprising human Vκ1-17 to Vκ2-24 gene segments, and a 5′ 60 kb region (“overlap region”) that comprises human Vκ3-25 to Vκ2-30 gene segment. Finally, the third large targeting vector (MAID6647, which is also derived from a vector constructed as described in U.S. Patent Application Publication No. 2012/0096512A1, incorporated by reference) comprises a 5′ 60 kb overlap region comprising human Vκ3-25 to Vκ2-30, a 90 kb sequence comprising Vκ3-31 to Vκ2-40 and an FRT-Ub-Neo-FRT selection cassette and a 15.5 kb 3′ mouse TCR A homology arm. The ES cells generated in FIG. 6 (MAID 6548, heterozygous for all human Jκ segments and four functional human Vκ gene segments) are electroporated with the three large targeting vectors described above, along with a nucleic acid encoding a modified Zinc Finger Nuclease (ZFN) that targets the hygromycin gene at nucleotide sequence TGCGATCGCTGCGGCCGAtcttagCCAGACGAGCGGGTTCGG (with cleavage site in lower case letters; SEQ ID NO:76) and promotes double stranded breaks at the Hyg sequence. The three co-electroporated large targeting vectors are inserted by homologous recombination into the DNA sequence replacing the region containing and surrounding the Hyg selection cassette. The resulting ES cells contain at the endogenous TCRα locus a human immunoglobulin variable domain comprising human Jκ1 to Jκ5 and Vκ4-1 to Vκ2-40 gene segments (i.e., all functional human Vκ gene segments of the proximal Vκ cluster). Successful incorporation of the three large targeting vectors is confirmed using the TAQMAN® assays described above (Lie and Petropoulos, supra), using probes and primers indicated in FIG. 9 and listed in Table 6 below (GOA=gain of allele; LOA=loss of allele; copy number=check for copy number of sequence to trace transgenic integration vs. targeted integration; hArm1=30 kb 3′ homology arm of the first large targeting vector (MAID 1710); hArm2=20 kb overlap of the first (MAID 1710) and the second (MAID 6600) large targeting vectors, mArm=15.5 kb 5′ homology arm of the second targeting vector (MAID 6600), hArm3=60 kb overlap of the second (MAID 6600) and the third (MAID6647) targeting vectors, WT mouse control-sequences present at the mouse TCRα locus).

TABLE 6
TAQMAN Primers and Probes

| Gene | Assay | Fwd Primer | Probe | Rev Primer |
|---|---|---|---|---|
| HYG | LOA | TGCGGCCGATC | ACGAGCGGGTTCG | TTGACCGATTC |
| | | TTAGCC (SEQ ID | GCCCATTC (SEQ ID | CTTGCGG (SEQ |
| | | NO: 77) | NO: 78) | ID NO: 79) |
| HYG-U | LOA | CGACGTCTGTC | AGTTCGACAGCGTG | CACGCCCTCCTA |
| | | GAGAAGTTTCTG | TCCGACCTGA (SEQ | CATCGAA (SEQ |
| | | (SEQ ID NO: 80) | ID NO: 81) | ID NO: 82) |
| Hyg-D | LOA | TGTCGGGCGTAC | CCGTCTGGACCGAT | GGGCGTCGGTTT |
| | | ACAAATCG (SEQ | GGCTGTGT (SEQ ID | CCACTATC (SEQ |
| | | ID NO: 83) | NO: 84) | ID NO: 85) |
| hIgK6 | Copy | GTCAAGCACTGC | AACCCTTGTGCTAT | TGTTGTAGACCC |
| | number of | TGGCACAC (SEQ | TGAATTGCTATGCT | TCCGCCAC (SEQ |
| | hArm1 | ID NO: 86) | GTCAG (SEQ ID | ID NO: 88) |
| | | | NO: 87) | |
| hIgK12 (MAID | GOA | TTGCCTTTCTCA | CAGCCCATCCTGTC | TGGCCCAACAGT |
| 1710 insert) | | CACCTGCAG | ACTTCGCTGGA | ACAGCTCAG |
| | | (SEQ ID NO: 89) | (SEQ ID NO: 90) | (SEQ ID NO: 91) |
| hIgK13 | Copy | TCAGTCAATCAC | TCCCCAGGTAGCCT | CACATTACTGAG |
| | number of | CTTTCCCAGC | CATGAACCAATGTT | TCCCCACAGGG |
| | hArm2 | (SEQ ID NO: 92) | (SEQ ID NO: 93) | (SEQ ID NO: 94) |
| hIgK14 | Copy | CATTGTCAAAGA | ACCATTGCAGTTTA | TCTTGCAATGGG |
| | number of | AGCACTGGAAAT | CCCACGGTTAGGAT | ATCATCAGATG |
| | hArm2 | G (SEQ ID NO: 95) | TTTT (SEQ ID NO: 96) | (SEQ ID NO: 97) |
| Neo | GOA | GGTGGAGAGGC | TGGGCACAACAGA | GAACACGGCGG |
| | | TATTCGGC (SEQ | CAATCGGCTG (SEQ | CATCAG (SEQ ID |
| | | ID NO: 98) | ID NO: 99) | NO: 100) |
| hIgK15 | GOA | CAGGTGCAAAG | TGGGTCCTGCCCAT | GGCAGCCTGAGT |
| | | GTGACCACAG | CCATGCA (SEQ ID | GTCAGAGC (SEQ |
| | | (SEQ ID NO: 101) | NO: 102) | ID NO: 103) |
| hIgK25 | GOA | GTTCAGGCCCCA | TCCTCTCTGGAGCA | CCTGAAGCCATG |
| | | CAGACTCTC | ACCATGAAGTTCCC | AGGGCAG (SEQ |
| | | (SEQ ID NO: 104) | T (SEQ ID NO: 105) | ID NO: 106) |
| TCRA Arm4 | Copy | GCGCCACATGAA | TGTACCCAATCTTC | GGCATCCTGTCC |
| | number of | TTTGACCAG | CAAAGAAAGAGCT | TCCCTTC (SEQ ID |
| | mArm | (SEQ ID NO: 107) | G (SEQ ID NO: 108) | NO: 109) |
| Parental | WT mouse | CAGTAAGGGAA | TGCACACTGCTCAC | TGCTGGTGGCCC |
| 1540m1 | control | GAGACTACAAC | CACTGCAAGCTAT | CATCT (SEQ ID |
| | | AGCAT (SEQ ID | (SEQ ID NO: 111) | NO: 112) |
| | | NO: 110) | | |
| Parental | WT mouse | GAACTCAGCTAT | CAGCCCAGCAGCTG | GCTCAGGGAGA |
| 1540m3 | control | GATAGTGTCGAA | TGGGTTCTC (SEQ | ACACAGAACTTA |
| | | TGTA (SEQ ID | ID NO: 114) | GA (SEQ ID |
| | | NO: 113) | | NO: 115) |
| hIgK5 | MAID 6548 | CCCCGTCCTCCT | TCATGTCCATTAAC | TGCAAGTGCTGC |
| | sequence | CCTTTTTC (SEQ | CCATTTACCTTTTG | CAGCAAG (SEQ |
| | (see Table | ID NO: 116) | CCCA (SEQ ID | ID NO: 118) |
| | 2) | | NO: 117) | |
| hIgK22 | Copy | TGGCTCCAAGAA | CCCTGACTTTGCTG | GGTCCAGTGGAA |
| | number of | CAGTTTGCC | CTCAACTCACAGCC | TCTGCCATG |
| | hArm3 | (SEQ ID NO: 119) | (SEQ ID NO: 120) | (SEQ ID NO: 121) |
| hIgK21 | GOA | CATTTGGCTACA | CCTGAGCCAGGGA | ACATGGCTGAGG |
| | | TATCAAAGCCG | ACAGCCCACTGATA | CAGACACC (SEQ |
| | | (SEQ ID NO: 122) | (SEQ ID NO: 123) | ID NO: 124) |
| hIgK26 | GOA | TGGGCCGTTATG | TGGCTTTACCCCTT | CACAGCTGAAGC |
| | | CTAGTACCA | TTGAAGGGCCC | AGGATGAGC |
| | | (SEQ ID NO: 125) | (SEQ ID NO: 126) | (SEQ ID NO: 127) |
| hIGK30 | GOA | TCTCTGAGCAGC | TTCTCCTTTGGTGT | ACCAGGCATGGC |
| | | CATCCCC (SEQ | AGAGGGCACCAGC | AGAAAGG (SEQ |
| | | ID NO: 128) | (SEQ ID NO: 129) | ID NO: 130) |

The resulting targeted locus in ES cells have the following junction sequences, where mouse sequences are in parentheses, human sequences are in normal font, multiple cloning sites are bolded, and Frt sequences are italicized (Table 7).

TABLE 7
Junction Sequences of Locus Resulting from Triple
ES Cell Targeting (5′ to 3′)

| | Seq ID | |
|---|---|---|
| Junction | No | Sequence |
| mouse Tcra/ | 131 | (GTCTTTTTTGTTCTTCACAGTTGAGCT |
| 5′ Frt | | TCATCAAAGTCACATGGGTTAAACTCTA |
| | | TGGAGTAGTCAGAACACACTCTTCA)GA |
| | | AGGGACTCCTGATTTCAAAGGGTACCGA |
| | | AGTTCCTATTCCGAAGTTCCTATTCTCT |
| | | AGAAAGTATAGGAACTTC |
| 3′ Frt/ | 132 | GAAGTTCCTATTCCGAAGTTCCTATTCTC |
| human IgK | | TAGAAAGTATAGGAACTTCCTAGGGTTTC |
| | | ACCGGTGGCGCGCCTGAGTAGTGCTTTAG |
| | | GTGTGTAATCACCAAAGATTTAGTGAAGT |
| | | CCCTGTGCAAGGAG |
| Human IgK/ | | Same as in Table 1 |
| mouse Tcra | | |

In yet other alternative strategy, the triple, double or single targeting of successive additional human Ig Vκ gene segments into the locus depicted in FIG. 6 may be accomplished using triple (three large targeting vectors), double (two large targeting vectors), or single (one large targeting vector) targeting schemes that involve zinc finger nuclease- or CRISPR-mediated destruction of a selection (e.g., hygromycin) cassette.

Targeted ES cells described above were used as donor ES cells and introduced into an 8-cell stage mouse embryo by the VELOCIMOUSE® method (see, e.g., U.S. Pat. No. 7,294,754 and Poueymirou et al. (2007) F0 generation mice that are essentially fully derived from the donor gene-targeted ES cells allowing immediate phenotypic analyses Nature Biotech. 25(1):91-99). VELOCIMICE® (F0 mice fully derived from the donor ES cell) independently bearing a chimeric human Igκ V-mouse Tcra C gene were identified by genotyping using a modification of allele assay that detects the presence of the unique gene sequences.

### Example 2.2

### Construction of Chimeric Human IgH Variable-Mouse TCRB Constant Locus

Chimeric human IgH variable-mouse TCRB constant loci are constructed by one of several different strategies.

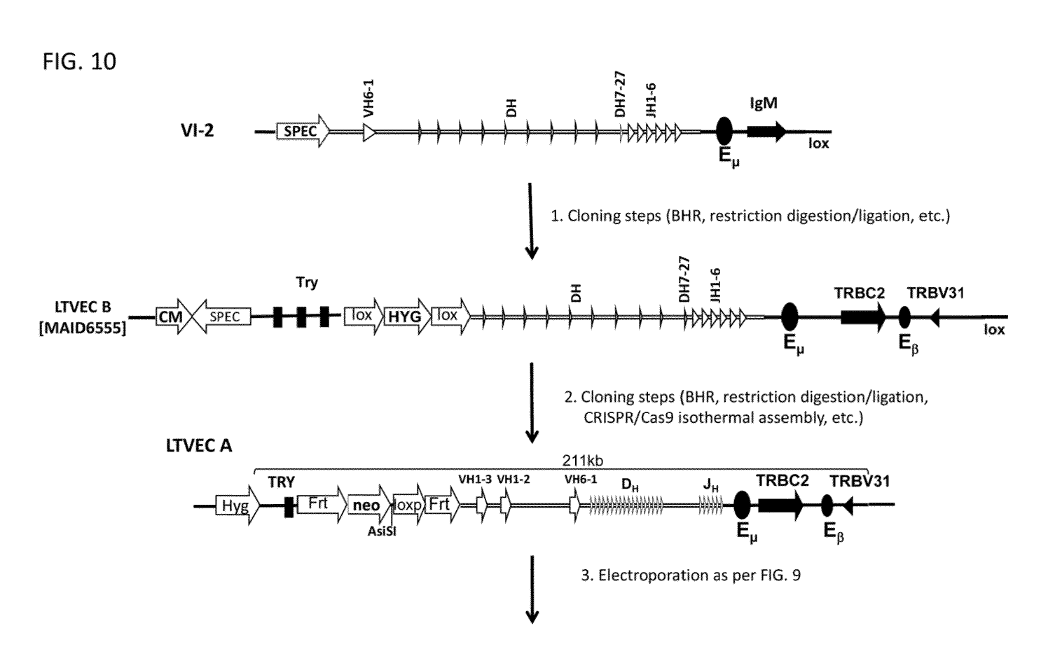

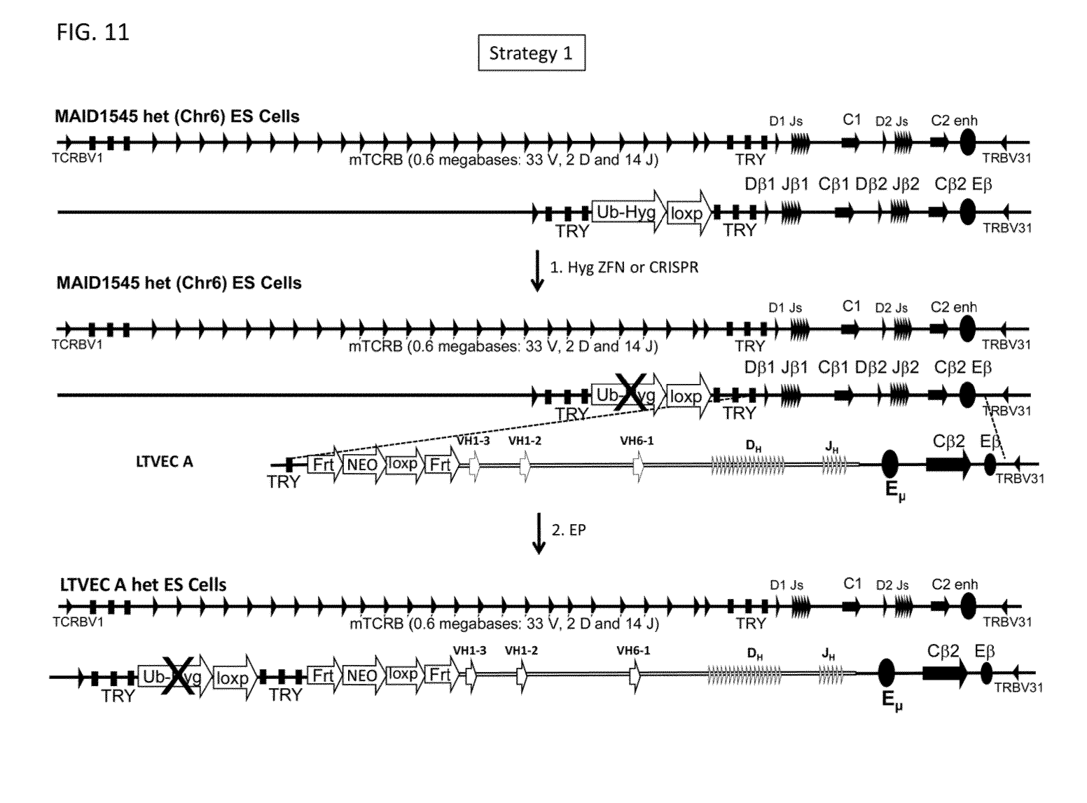

Strategy 1 is depicted in FIG. 11. In order to obtain large targeting vector (LTVEC A) for use in this strategy, a VELOCIMMUNE® immunoglobulin heavy chain LTVEC comprising all human immunoglobulin heavy chain J_{H} and D_{H} segments and one proximal V_{H} segment (“VI-2”) was modified in several BHR and restriction digestion/ligation steps as depicted in FIG. 10 to generate a construct comprising all human immunoglobulin J_{H} and D_{H} segments (LTVEC B [MAID 6555]). The 5′ mouse arm of the LTVEC B contained trypsinogen genes (Try15-Try20), while the 3′ arm contained the mouse TCRB C2 and Vβ31 genes. LTVEC B also contained the mouse IgM enhancer (Eμ) for enhancement of immunoglobulin heavy chain variable region recombination at the CAR locus.

In the next step, LTVEC B was further modified via several steps of BHR, restriction digestion/ligation, and CRISPR/Cas9-mediated isothermal BAC assembly (U.S. patent application Ser. No. 14/747,461, filed Jun. 23, 2015, incorporated herein by reference) to generate a large targeting vector (LTVEC A) comprising, from 5′ to 3′: (1) an Em7-Hyg cassette for selection in E. coli; (2) a 20 kb 5′ mouse arm for homologous recombination in ES cells containing a trypsinogen gene (Try20, genome location 6:41504907-41525442); (3) an Frt-Neo-loxP-Frt cassette for selection in E. coli or ES cells; (4) 145 kb of the human IgH locus containing the 3 most proximal VH segments and all of the DH and JH segments; (5) 1296 bp of the mouse IgH locus containing the IgM enhancer (Eμ) (genome location 12:113427167-113428462) (alternatively, this sequence is excluded); and (6) a 40 kb 3′ mouse arm for homologous recombination in ES cells containing the Trbc2 constant gene (genome location 6:41543957-41584559) (see FIG. 10).

LTVEC A has the following junction sequences, where mouse sequences are in parentheses, human sequences are in normal font, multiple cloning sites are bolded, and Frt sequences are italicized (Table 8):

TABLE 8
Junctional sequences of IgH V-TCRB C Large
Targeting Vector (LTVEC A) (5′ to 3′)

| Junction | Seq ID No | Sequence |
|---|---|---|
| mouse Tcrb (5′Try | 133 | (ATAATAATTAATAATAATAAATAGTAAATTT |
| repeat end)/Frt | | CTGTAGAATCATAATGAGG)TCTAGACCCCCG |
| | | GGCTCGATAACTATAACGGTCCTAAGGTAG |
| | | CGGTACCGAAGTTCCTATTCCGAAGTT |
| Frt/human IgH | 134 | TCTAGAAAGTATAGGAACTTCCTAGGGTTTCA |
| (V, D, J | | CCGGTGGCGCGCCGAGCTTTCTGGTTCAGCC |
| portion) | | AGGGACACAGAACCAGGAAGACATCGTGGCTT |
| | | TTCTA |
| human IgH (V, D, J | 135 | CTTTGGAAAATGGGACTCAGGTTGGGTGCGTC |
| portion)/mouse IgH | | TGATGGAGTAACTGAGCCTCTAGA(CTGAGCA |
| (Eμ portion) | | TTGCAGACTAATCTTGGATATTTGTCCCTGAG |
| | | GGAGCCGGCTG) |
| mouse IgH (Eμ | 136 | (AAACTTCTTAAAATTACTCTATTATTCTTCCC |
| portion)/ | | TCTGATTATTGGTCTCCA)CTCGAGTGCCATT |
| mouse TCR B | | TCATTACCTCTTTCTCCGCACCCGACATAGA |
| (TCR C | | TAAAGCTT(GGAGACAGCTCTCAACTTCACCC |
| containing end) | | TTTCTGGGGGAGCGGGATGAAAAGGGA) |

In Strategy 1, following the cloning steps to generate LTVEC A, the chimeric locus was introduced into ES cells in a single targeting step. As depicted in FIG. 11, human IgH V, D, and J segments were inserted downstream of the mouse 3′ trypsinogen (TRY) genes (mouse TRY genes are not depicted to scale; the TCR B locus contains a number of TRY genes) and upstream of mouse TCRB C2 in ES cells bearing a TCRB locus comprising deletion of all mouse TCRB V segments between the two trypsinogen repeats (MAID 1545, see FIG. 8A of U.S. Pat. No. 9,113,616, incorporated herein by reference). Thus, mouse TCRB D1-J1-C1 and D2-J2 were replaced with the human V, D, and J segments, while the majority of mouse V segments were deleted. The mouse IgM enhancer (Eμ) was also inserted 5′ of TCRB C2, but it may also be deleted, e.g., in a targeting vector used for electroporation, using methods known in the art. Optionally, the mouse TCR Vβ31 gene may also be deleted.

To make additional insertions of human IgH V_{H} segments, the Hyg gene in MAID 1545 is inactivated (see also FIG. 11). This can be done either before or after targeting by: (1) introducing a small indel mutation into the Hyg coding sequence using CRISPR/Cas9 or zinc finger nuclease (ZFH) so that a functional Hyg protein can no longer be made (See U.S. patent application Ser. No. 14/731,914, filed Jun. 5, 2015, incorporated herein by reference); or (2) replacing with Loxp-Neo-Loxp cassette by homologous recombination and subsequently removing the cassette with Cre.

The junction sequences of the resulting CAR locus are the same as listed in Table 8 above. Neo-resistant ES cells are screened by TAQMAN® assay to identify correctly targeted clones. Since the 1545 allele contains an upstream Hyg-Loxp cassette, Loxp site in LTVEC A allows determination of which TCRB allele is targeted in 1545het ES cells; therefore, Cre deletion of the region between the two Loxp sites is used to determine which clones are targeted to the 1545 allele as opposed to the wild-type Tcrb allele.

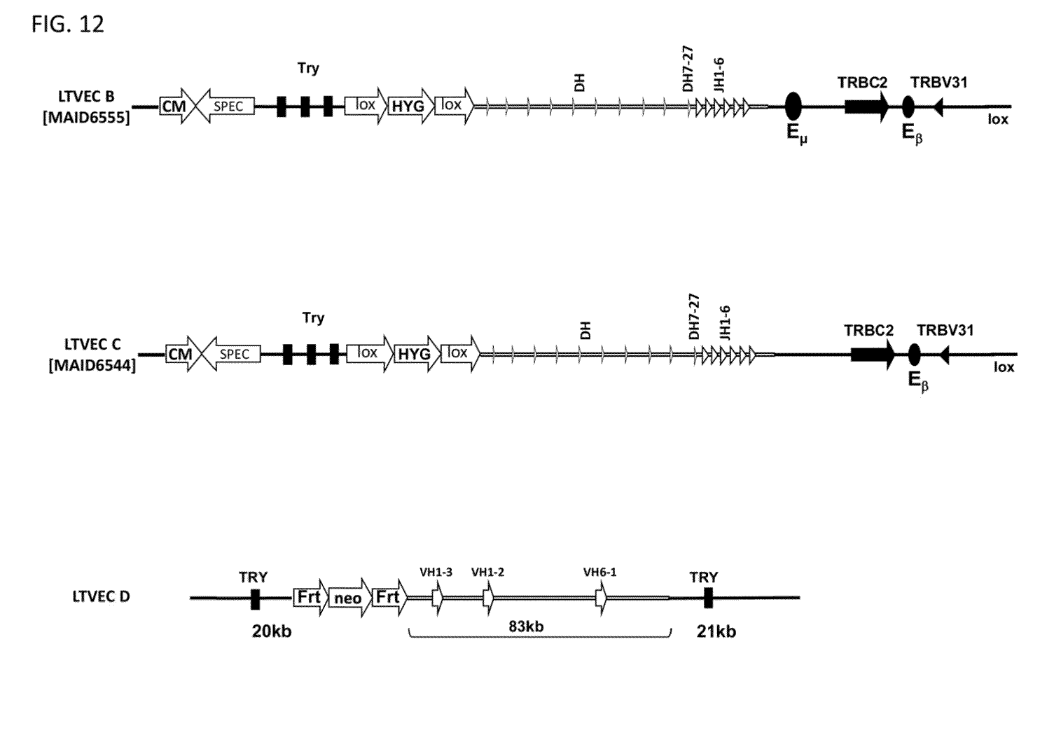

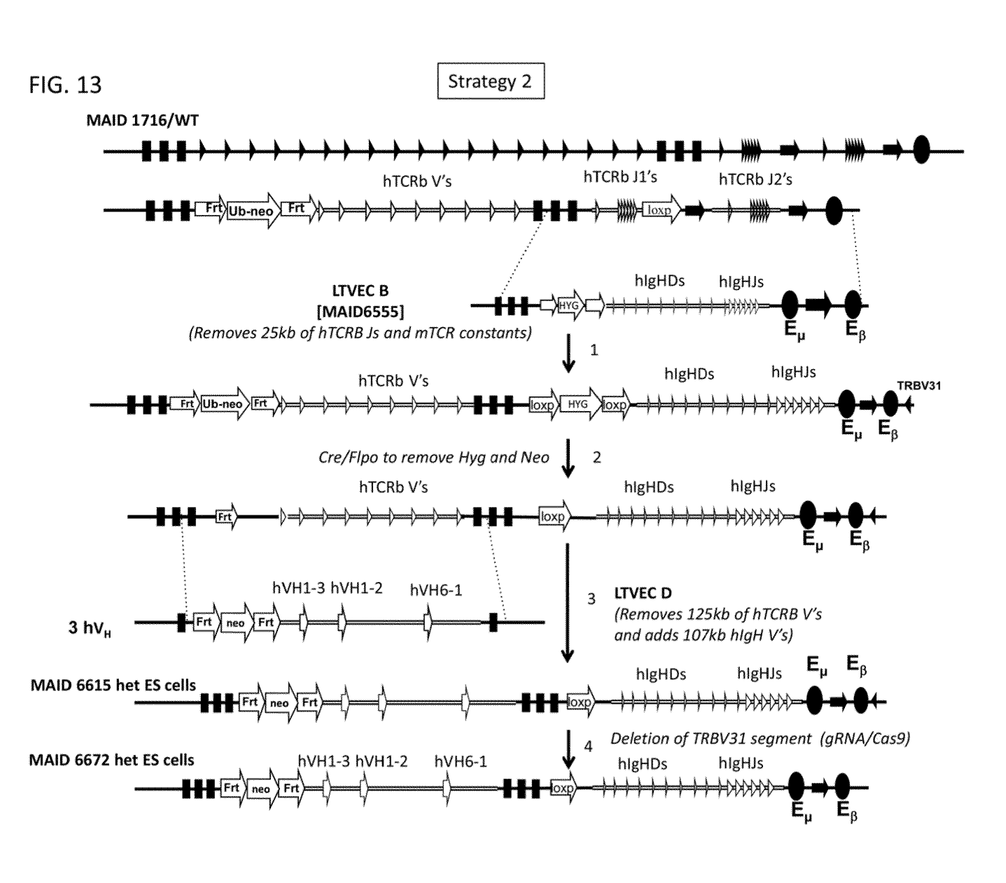

In Strategy 2, the basic organization of the Tcrb locus (V segments between the 5′ and 3′ Try gene clusters, and D and J segments between the 3′ Try gene cluster and the Tcrb2 constant) is preserved. Specifically, ES cells comprising 14 human TCRB V segments and all human TCRB D and J segments (see FIG. 7 of U.S. Pat. No. 9,113,616, incorporated herein by reference) were modified by first replacing the region comprising human TCRB D and J segments with immunoglobulin heavy chain D and J segments utilizing a large targeting vector (LTVEC B, see FIG. 12) comprising from 5′ to 3′: (1) a 5′ homology arm comprising mouse Try genes (mouse Try genes are depicted not to scale; the TCR B locus contains a number of Try genes), (2) a HYG selection cassette, and (3) all human immunoglobulin heavy chain D and J segments, (4) mouse Eμ gene sequence, and (5) 3′ homology arm comprising mouse TCR B constant region, mouse Eβ gene, and mouse TCR Vβ31 gene segment (step 1 of FIG. 13)

Following this modification, and the removal of the selection cassettes, ES cells were further modified by electroporation with a large targeting vector (LTVEC D, see FIG. 12) comprising from 5′ to 3′: (1) a 5′ homology arm comprising a mouse Try gene (Try7), (2) a NEO selection cassette, (3) three human immunoglobulin heavy chain variable gene segments, and (4) a 3′ homology arm comprising a mouse Try gene (Try4) (see step 3 of FIG. 13).

The resulting ES cells comprise human immunoglobulin heavy chain V gene segments V_{H}1-3, V_{H}1-2, and V_{H}6-1, all human immunoglobulin heavy chain D and J gene segments, as well as mouse immunoglobulin Eμ enhancer, mouse TCR B constant region, mouse TCR B enhancer, and a distal 3′ mouse TCR Vβ31 gene segment. At each step in the strategy, successful introduction of a particular LTVEC was confirmed using a TAQMAN® assay as described above.

The final TCR B locus in the ES cells contained the following junction sequences, where mouse sequences are in parentheses, human sequences are in normal font, multiple cloning sites are bolded, and Frt sequences are italicized:

TABLE 9
Junction sequences of the TCR B CAR locus
of Strategy 2 (5′ to 3′)

| | Seq ID | |
|---|---|---|
| Junction | No | Sequence |
| mouse TCR B | 137 | (GGGGGGGTGGGGTGGAGGAGGAGGGTACAGCATCTCCTCTCCTTC |
| (5′ Try | | CTCTC)TGGTACCGAAGTTCCTATTCCGAAGTTCCTATTCTCTAGAAAGT |
| repeat end)/ | | ATAGGA |
| Frt | | |
| Frt/human | 138 | TATTCTCTAGAAAGTATAGGAACTTCCTAGGGTTTCACCGGTGCGAT |
| IgH (V | | CGCATATCCATGTGTGTCCATTCTGGTTCAGCCAGGGACACAGAAC |
| segments) | | CAGGAAG |
| human IgH | 139 | CAGGCTTGCAGTCCTGGGCAGACTCCGTCACCTCTCTATGCCTCAGC |
| (V segments)/ | | CTTGGCGCGCC(TTTCAAATTGTTGTTGAGTTCAAAGTGGGCAACA |
| mouse TCR B | | GAAAAGGGGGTGTGAG) |
| (3′ Try | | |
| repeat end) | | |
| mouse TCR B | 140 | (AATAATTAATAATAATAAATAGTAAATTTCTGTAGAATCATAATGA |
| (3′ Try | | GG)TCTAGACCCCCGGGCTCGATAACTATAACGGTCCTAAGGTA |
| repeat | | GCGAACCGGTATAACTTCGTATAAGGTATCCTATACGAAGTTATCTCG |
| end)/lox2372/ | | AGGGGGGGCCCGGTACCGATTCAATGTCCACACCCGGGGCTGGA |
| human IgH | | GCGTAGCCATGAGCCACGC |
| (D and J | | |
| segments) | | |
| human IgH | 141 | CTTTGGAAAATGGGACTCAGGTTGGGTGCGTC |
| (D and J | | TGATGGAGTAACTGAGCCTCTAGA(CTGAGCA |
| segments)/ | | TTGCAGACTAATCTTGGATATTTGTCCCTGAG |
| mouse IgH | | GGAGCCGGCTG) |
| (Eμ portion) | | |
| mouse IgH | 142 | (AAACTTCTTAAAATTACTCTATTATTCTTCCC |
| (Eμ portion)/ | | TCTGATTATTGGTCTCCA)CTCGAGTGCCATT |
| mouse TCR B | | TCATTACCTCTTTCTCCGCACCCGACATAGA |
| (TCR C | | TAAAGCTT(GGAGACAGCTCTCAACTTCACCC |
| containing | | TTTCTGGGGGAGCGGGATGAAAAGGGA) |
| end) | | |

As an alternative of the Strategy 2 described above, instead of introduction of LTVEC B, LTVEC C (see FIG. 12), which does not comprise a mouse Eμ, was introduced. Additional strategies for generating chimeric TCR B CAR locus are described in the provisional applications, U.S. Patent Application Nos. 62/052,947, 62/076,836, 62/094,603, 62/167,650, incorporated herein by reference. Finally, as depicted in FIG. 13, a distal 3′ TCR Vβ31 can be deleted using various strategies, including using CRISPR/Cas9 technology.

Any selection cassettes remaining may be removed using either Cre or Flpo enzymes (see, e.g., FIG. 13). If desired for either Strategy 1 or Strategy 2, additional human V_{H} segments are added to the TCR variable region locus using LTVECs having a 5′ mouse Tcrb homology arm described above linked to human IgH sequences that overlap with the initial insertion.

Targeted ES cells described above were used as donor ES cells and introduced into an 8-cell stage mouse embryo by the VELOCIMOUSE® method. VELOCIMICE® (F0 mice fully derived from the donor ES cell) independently bearing a chimeric human IgH V-mouse Tcrb C gene were identified by genotyping using a modification of allele assay that detects the presence of the unique gene sequences.

### Example 2.3

### Construction of Chimeric Antigen Receptor Mice

Mice bearing chimeric human Igκ V-mouse Tcra C gene and chimeric human IgH V-mouse Tcrb C gene are bred together to generate mice comprising both chimeric loci. Mice comprising both such chimeric loci express on their T cell surface a chimeric antigen receptor (CAR) comprising a T cell receptor constant domain and an immunoglobulin variable domain (a mouse T cell receptor constant domain and human immunoglobulin variable domain). Progeny are bred to homozygosity with respect to each chimeric gene.

Alternatively, ES cells comprising either chimeric human Igκ-mouse Tcra C gene or chimeric human IgH V-mouse Tcrb C gene are used to introduce a targeting vector comprising the other chimeric gene (chimeric human IgH V-mouse Tcrb C gene or chimeric human Igκ-mouse Tcra C gene, respectively), and mice carrying both chimeric genes are generated from these ES cells via VELOCIMOUSE® method as described above.

Expression of chimeric human Igκ-mouse Tcra C/chimeric human IgH V-mouse Tcrb C antigen receptors is detected on the cell surface. One method for detection combines: (1) FACS analysis to detect TCR constant region expression (anti-TCR alpha antibody F1 (3A8) #TCR1145, Thermo-Pierce; anti-TCR beta antibody F1 (8A3) #TCR1151, Thermo-Pierce; anti-TCR alpha-beta heterodimer antibody clone T10B9.1A-31, BD-Pharmigen; anti-TCR alpha-beta heterodimer antibody clone IP26; eBioscience) using standard techniques, with (2) Western blotting to confirm the size of the chimeric proteins using the same antibodies, and with (3) RT-PCT using forward primer combinations that anneal to the immunoglobulin variable segment sequences and a primer that anneals to the TCR constant region sequence to confirm the expression of the chimeric transcripts. Additionally, a combination of anti-CD3 and anti-TCR alpha-beta antibodies can be used to confirm the formation of TCR/CD3 complex on the cell surface. Next-generation sequencing as described above is also used to confirm expression of chimeric transcripts.

### Example 3

### Human Igκ Variable Region Segment Usage in T Cells in Mice Harboring an Igκ/TCRα Chimeric Antigen Receptor Locus

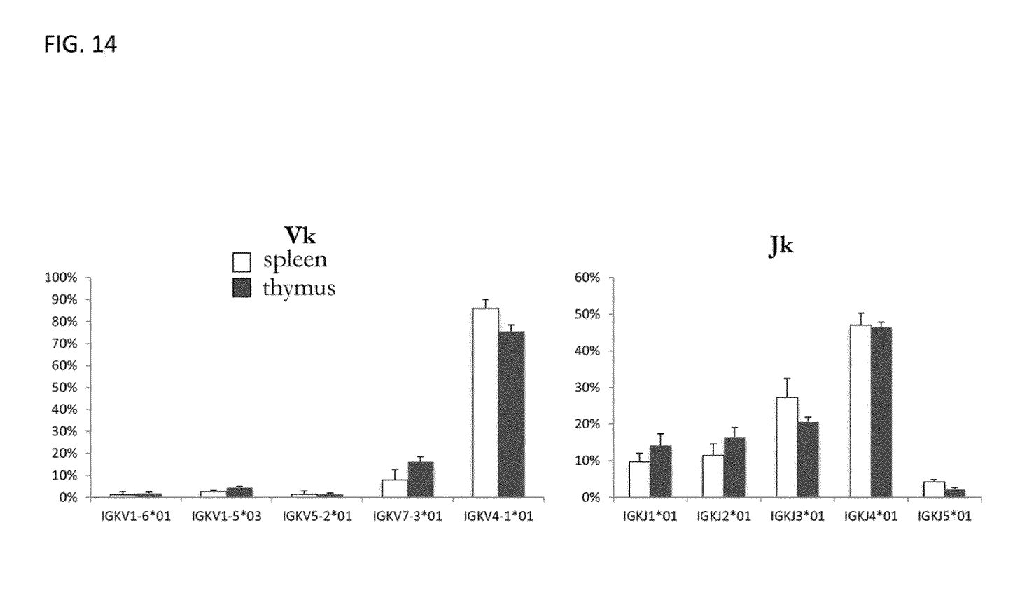

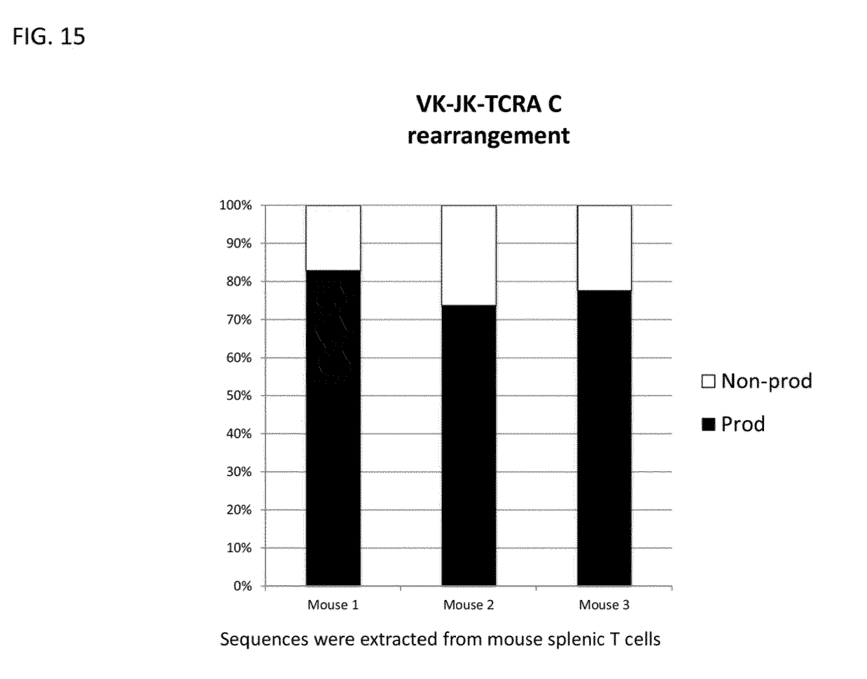

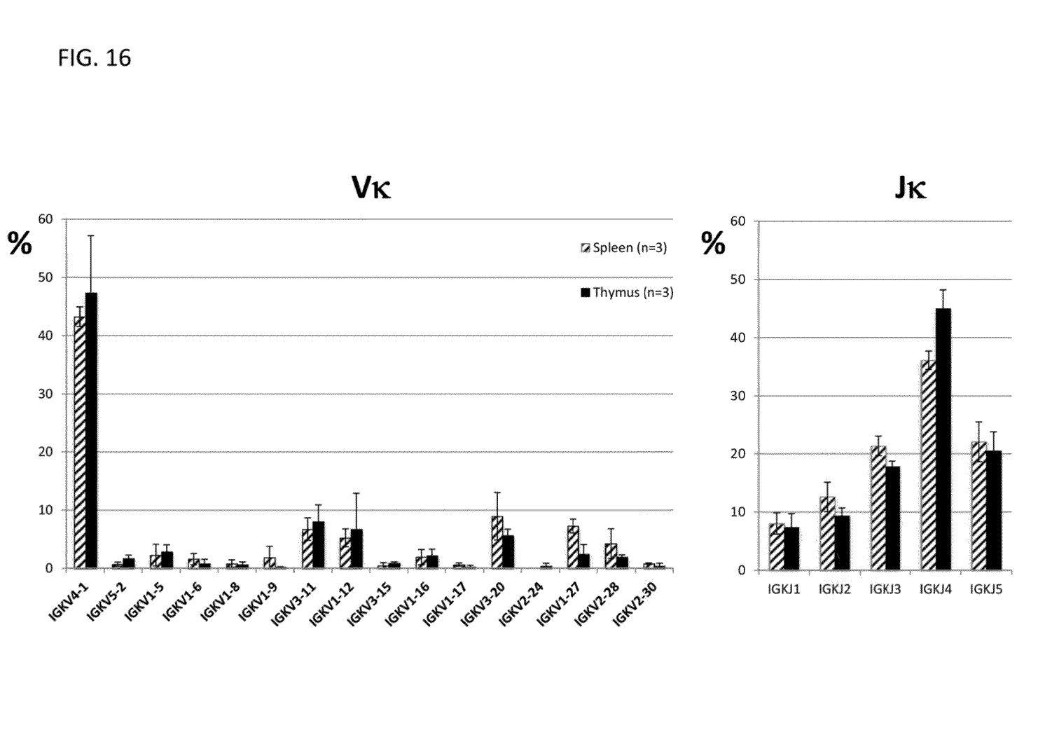

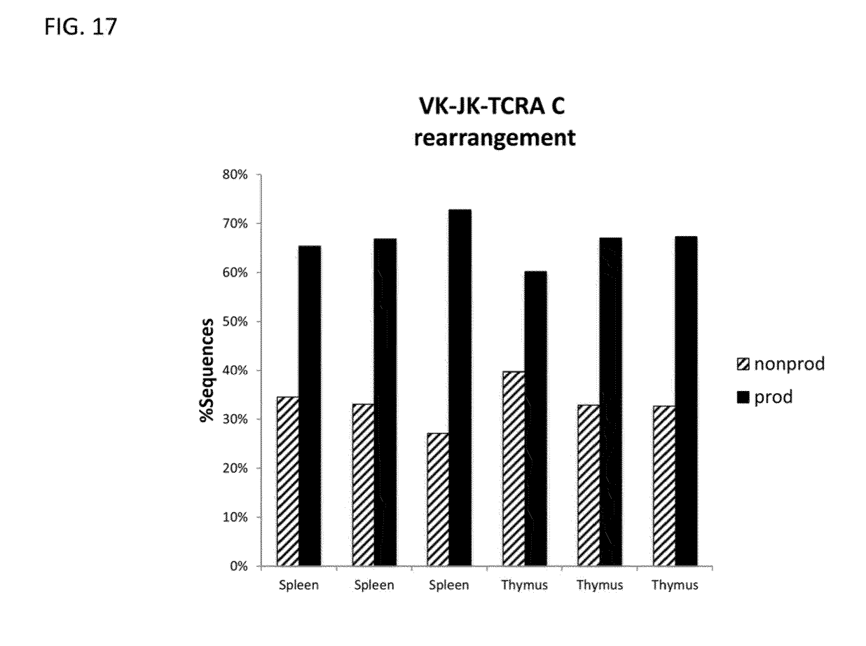

Thymocytes and splenocytes were harvested from three mice comprising in their genome a CAR locus in which a TCRα variable region was replaced by a partial human Igκ variable region (4 functional V_{κ} and 5 functional J_{κ} in FIGS. 14 and 15—see mice generated as depicted in FIG. 6; 16 functional V_{κ} and 5 functional J_{κ} in FIGS. 16 and 17—see mice generated as depicted in FIG. 7). T cells were positively enriched from total splenocytes by magnetic cell sorting using anti-CD90.2 magnetic beads and MACS® columns (Miltenyi Biotech). Total RNA was isolated from the purified splenic T cells and thymocytes using an RNeasy Plus RNA isolation kit (Qiagen) according to manufacturer's instructions.

Reverse transcription was performed to generate cDNA containing TCRα constant region sequence, using a SMARTer™ RACE cDNA Amplification Kit (Clontech) and a TCRα specific primer (5′-TCAAAGTCGGTGAACAGGCAGAG-3; SEQ ID NO: 143). During this process, a DNA sequence (PIIA: 5′-CCCATGTACTCTGCGTTGATACCACTGCTT-3; SEQ ID NO: 144) was attached to the 3′ end of the newly synthesized cDNAs. The cDNAs were purified by the NUCLEOSPIN® Gel and PCR Clean-Up Kit (Clontech).

Purified cDNAs were then amplified by PCR using a PIIA specific primer (5′-GTGACTGGAGTTCAGACGTGTGCTCTTCCGATCTAAGCAGTGGTATCAACGCAGA GT-3; SEQ ID NO: 145) and a TCRα specific primer (5′-ACACTCTTTCCCTACACGACGCTCTTCCGATCTACAGCAGGTTCTGGGTTCTGGAT G-3; SEQ ID NO: 146). PCR products were separated on 2% agarose gels and fragments between 400-700 bp in length were isolated and purified using a gel extraction kit (Qiagen). These fragments were further amplified by PCR using following primers: 5′-AATGATACGGCGACCACCGAGATCTACACXXXXXXACACTCTTTCCCTACACGA CGCTCTTCCGATC-3′ and 5′-CAAGCAGAAGACGGCATACGAGATXXXXXGTGACTGGAGTTCAGACGTGTGCTC TTCCGATCT-3′ (SEQ ID NO: 147 and SEQ ID NO: 148, respectively; “XXXXXX” represents a 6 bp index sequences to enable multiplexing samples for sequencing). PCR products between 400 bp-600 bp were isolated, purified, and quantified by qPCR using a KAPA Library Quantification Kit (KAPA Biosystems) before loading onto a Miseq sequencer (Illumina) for sequencing.

For bioinformatic analysis, the resulting Illumina sequences were sorted based on the sample index perfect match and trimmed for quality. Overlapping mate-pairs were then assembled and annotated based on alignment of rearranged Igκ sequences to human germline V and J segments database and rearranged TCRα sequences to mouse germline V and J segments database using local installation of igblast (NCBI, v2.2.25+). A sequence was marked as ambiguous and removed from analysis when multiple best hits with identical score were detected. A set of perl scripts was developed to analyze results and store data in mysq1 database.

For mice comprising 4 functional V_{κ} and 5 functional J_{κ}, as shown in FIG. 14, sequence analysis revealed that the Ig κ variable domain in the CAR locus underwent VJ recombination in T cells and thymocytes of the CAR transgenic mouse, with ˜80% of reads containing the most proximal Vκ gene segment (IGVK4-1), which rearranged with different Jκ gene segments in both the spleen and thymus. As shown in FIG. 15, the majority rearranged human Igκ VJ sequences amplified from splenic T cells were productive.

For mice comprising 16 functional V_{κ} and 5 functional J_{κ}, as shown in FIG. 16, sequence analysis revealed that the Ig κ variable domain in the CAR locus underwent VJ recombination in splenic T cells and thymocytes of the CAR transgenic mouse. These rearrangements involved all functional human Vκ and Jκ segments, with ˜40% of reads containing the most proximal Vκ gene segment (IGVK4-1). As shown in FIG. 17, ˜⅔ of rearranged human Ig κ VJ sequences amplified from splenic T cells and thymus were productive.

### Example 4

### Human IgH Variable Region Segment Usage in T Cells in Mice Harboring an IgH/TCRβ Chimeric Antigen Receptor Locus

Thymocytes from four mice comprising in their genome a CAR locus in which a TCRβ variable region was replaced by a partial human IgH variable region (3 functional human VH and all functional human D and JH gene segments, See FIG. 13), and splenocytes from three mice comprising in their genome the same CAR (IgH+TCRCβ) locus, were harvested. T cells were positively enriched from total splenocytes by magnetic cell sorting using anti-CD90.2 magnetic beads and MACS® columns (Miltenyi Biotech). Total RNA was isolated from the purified splenic T cells and thymocytes using an RNeasy Plus RNA isolation kit (Qiagen) according to manufacturer's instructions.

Reverse transcription was performed to generate cDNA containing TCRβ constant region sequence, using a SMARTer™ RACE cDNA Amplification Kit (Clontech) and a TCRβ specific primer (5′-CGAGGGTAGCCTTTTGTTTGTTTGC-3; SEQ ID NO: 149). During this process, a DNA sequence (5′-CCCATGTACTCTGCGTTGATACCACTGCTT-3; SEQ ID NO: 150) was attached to the 3′ end of the newly synthesized cDNAs. The cDNAs were purified by the NUCLEOSPIN® Gel and PCR Clean-Up Kit (Clontech).

Purified cDNAs were then amplified by PCR using primers 5′-ACACTCTTTCCCTACACGACGCTCTTCCGATCTGACCTTGGGTGGAGTCACATTTC TC-3′ and 5′-GTGACTGGAGTTCAGACGTGTGCTCTTCCGATCTAAGCAGTGGTATCAACGCAGA GT-3′ (SEQ ID NOs: 151 and 152, respectively). These fragments were further amplified by PCR using following primers: 5′-AATGATACGGCGACCACCGAGATCTACACXXXXXXACACTCTTTCCCTACACGA CGCTCTTCCGATCT-3′ and 5′-CAAGCAGAAGACGGCATACGAGATXXXXXXGTGACTGGAGTTCAGACGTGTGCT CTTCCGATCT-3′ (“XXXXXX” is a six-nucleotide barcode sequence; (SEQ ID NOs: 153 and 154, respectively)). PCR products between 490-710 base pairs were isolated, purified, and quantified by qPCR using a KAPA Library Quantification Kit (KAPA Biosystems) before loading onto a Miseq sequencer (Illumina) for sequencing.

For bioinformatic analysis, the resulting Illumina sequences were demultiplexed and trimmed for quality. Overlapping paired-end reads were then assembled and annotated based on alignment of rearranged IgH sequences to human germline V, D and J segments database using local installation of igblast (NCBI, v2.2.25+). A sequence was marked as ambiguous and removed from analysis when multiple best hits with identical score were detected. A set of PERL scripts was developed to analyze results and store data in mysq1 database.

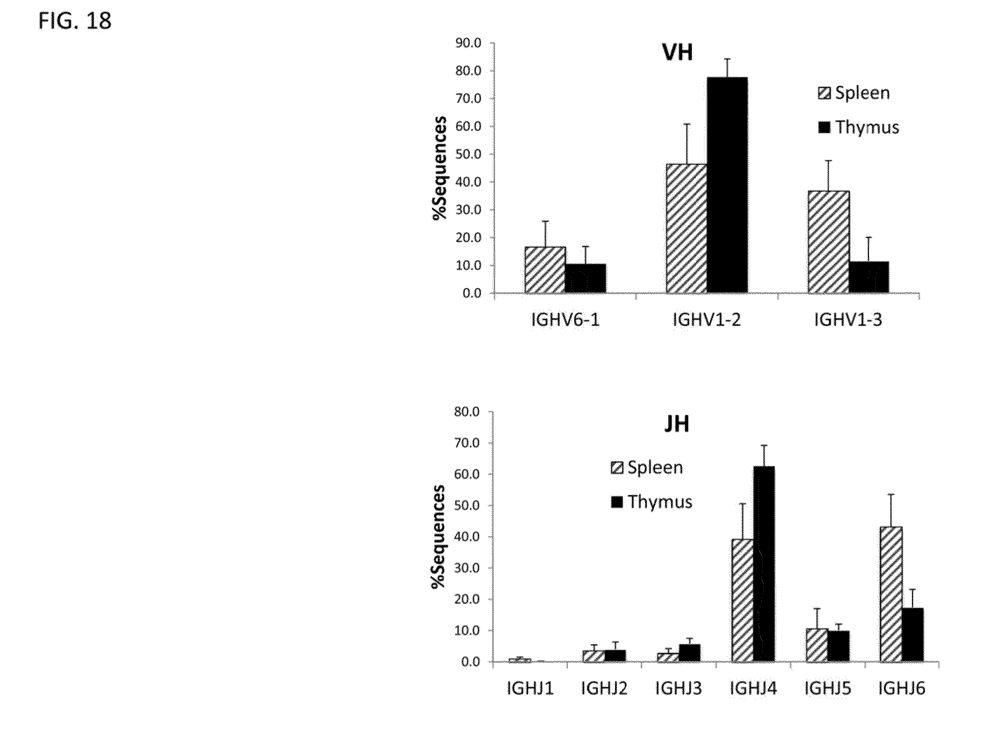

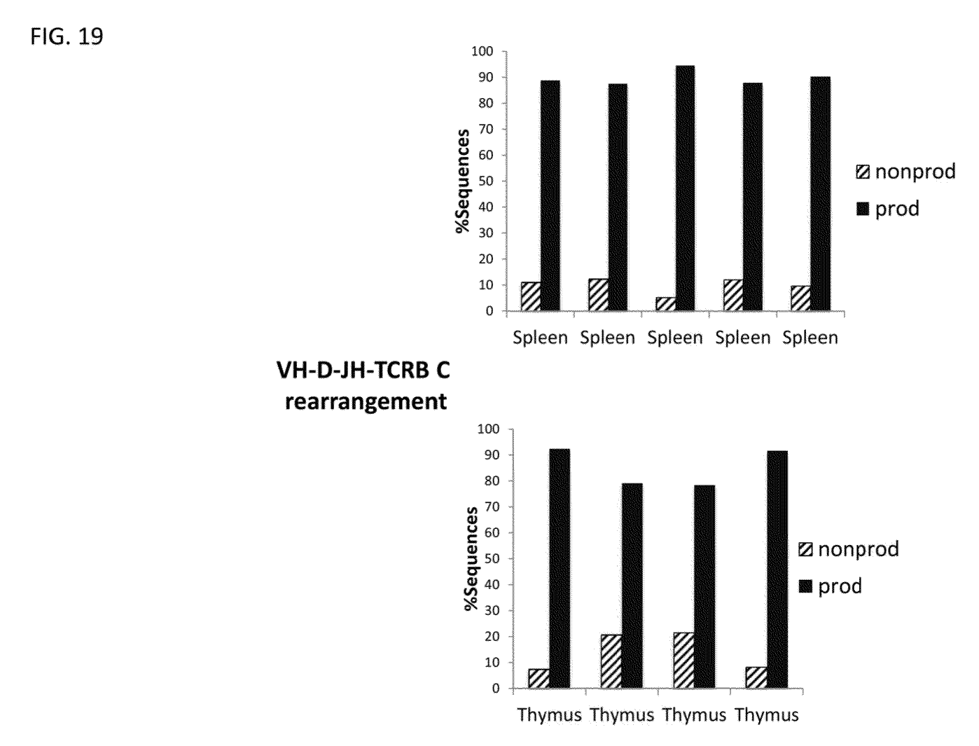

For mice comprising 3 functional human V_{H} and all functional human D and J_{H}, as shown in FIG. 18, sequence analysis revealed that the IgH variable region in the CAR locus underwent VDJ recombination in spleen and thymus of the CAR transgenic mouse. Analysis of V_{H} and J_{H} segments is shown. As shown in FIG. 19, the majority of rearranged human IgH VDJ sequences amplified from spleen or thymus were productive.

### Example 5

### Generation of Antigen Binding Proteins from Mice Harboring Chimeric Antigen Receptor Loci

After breeding mice that contain engineered chimeric antigen receptor loci human Igκ-mouse Tcra C and human IgH V-mouse Tcrb C as described above in Example 2, selected mice are immunized with an antigen of interest (e.g., an antigen that will be presented on MHC, such as a viral peptide-MHC antigen; tumor peptide-MHC antigen; self-autoimmune peptide-MHC antigen). Following antigen challenge, antigen-specific T cells are recovered from the animals by sorting with a labeled tertramerized version of immunogen. The sequences of Igκ and IgH variable regions of the sorted CART cells are determined and these variable region sequences are cloned in operable linkage upstream of the human TCRα and TCRβ constant regions, respectively. The chimeric nucleic acid sequences are introduced into reporter T cell lines. Reporter T cell lines are screened on target cells expressing the peptide-MHC complex used for immunization, and CARs having the desired property, e.g., affinity, selectivity, epitope, etc., for the antigen of interest are selected. The sequences of Igκ and IgH variable regions of the selected CAR are determined and these variable region sequences are cloned in operable linkage upstream of the human Igκ and IgH constant regions, respectively, for generation of human antibodies that are specific for the targeted peptide-MHC complex. These antibodies can be used to target infected or tumor cells that express the peptide-MHC of interest for destruction. Alternatively, if the peptide-MHC target is involved in inducing autoimmunity, these antibodies can be used to block the activation of autoimmune T-cells to alleviate the symptoms of disease. Additionally, the chimeric human CAR clone obtained from immunization of the CAR mouse described herein can be used, e.g., for introduction into a T cell obtained from a human patient for adaptive T cell transfer.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A genetically modified non-human animal comprising in its germline a chimeric antigen receptor (CAR) locus, the CAR locus comprising:
an unrearranged variable region locus comprising unrearranged immunoglobulin (Ig) variable region gene segments; and
a constant region locus comprising a T cell receptor (TCR) constant region;
wherein the human unrearranged Ig variable region gene segments are operably linked to the TCR constant region gene such that the genetically modified non-human animal expresses a CAR polypeptide comprising an Ig variable domain encoded by a rearranged Ig variable region gene derived from the unrearranged Ig variable region gene segments and a TCR constant domain encoded by the TCR constant region gene.

2. The genetically modified non-human animal of claim 1, wherein the unrearranged Ig variable region gene segments are human.

3. The genetically modified non-human animal of claim 1, wherein the TCR constant region gene is of endogenous species origin.

4. The genetically modified non-human animal of claim 1, wherein the TCR constant region gene is a mouse TCR constant region gene or a rat TCR constant region gene.

5. The genetically modified non-human animal of claim 1, wherein the unrearranged Ig variable region gene segments are human Ig heavy chain (IgH) variable region gene segments.

6. The genetically modified non-human animal of claim 1, wherein the unrearranged Ig variable region gene segments are human Ig light chain (IgL) variable region gene segments.

7. The genetically modified non-human animal of claim 6, wherein IgL variable region gene segments are human κ gene segments.

8. The genetically modified non-human animal of claim 6, wherein IgL variable region gene segments are λ gene segments.

9. The genetically modified non-human animal of claim 1, wherein the TCR constant region gene is a TCRα constant region gene.

10. The genetically modified non-human animal of claim 9, wherein the CAR locus is located at an endogenous TCRα locus.

11. The genetically modified non-human animal of claim 9, wherein the unrearranged human Ig variable region gene segments replace endogenous TCRα variable region gene segments.

12. The genetically modified non-human animal of claim 10, wherein the TCRα constant region gene is an endogenous TCRα constant region gene.

13. The genetically modified non-human animal of claim 1, wherein the TCR constant region gene is a TCRβ constant region gene.

14. The genetically modified non-human animal of claim 13, wherein the CAR locus is located at an endogenous TCRβ locus.

15. The genetically modified non-human animal of claim 14, wherein the unrearranged human Ig variable region gene segments replace endogenous TCRβ variable region gene segments.

16. The genetically modified non-human animal of claim 15, wherein the TCRβ constant region gene is an endogenous TCRβ constant region gene.

17. The genetically modified non-human animal of claim 1, wherein the non-human animal is a rodent.

18. The genetically modified non-human animal of claim 18, wherein the rodent is a mouse.

19. A genetically modified non-human animal comprising in its germline a first CAR locus and a second CAR locus,
the first CAR locus comprising a first unrearranged variable region locus comprising unrearranged immunoglobulin (Ig) V_{H}, D_{H} and J_{H} gene segments and a first constant region locus comprising a T cell receptor 13 (TCRβ) constant region gene, wherein the unrearranged Ig V_{H}, D_{H} and J_{H} gene segments are operably linked to the TCRβ constant region gene such that the genetically modified non-human animal expresses a first CAR polypeptide chain comprising an Ig heavy chain variable domain encoded by a rearranged heavy chain variable region gene derived from the unrearranged Ig V_{H}, D_{H} and J_{H} gene segments and a TCRβ constant domain encoded by the TCRβ constant region gene, and
the second CAR locus comprising a second unrearranged variable region locus comprising unrearranged Ig V_{κ} and J_{κ} gene segments and a second constant region locus comprising a T cell receptor α (TCRα) constant region gene, wherein the unrearranged Ig V_{κ} and J_{κ} gene segments are operably linked to the TCRα constant region gene such that the genetically modified non-human animal expresses a second CAR polypeptide chain comprising an Ig κ variable domain encoded by a rearranged Ig κ variable region gene derived from the unrearranged Ig V_{κ} and J_{κ} gene segments and a TCRα constant domain encoded by the TCRα constant region gene,
wherein the genetically modified non-human animal expresses a CAR comprising the first CAR polypeptide chain and the second CAR polypeptide chain.

20. The genetically modified non-human animal of claim 20, wherein the Ig V_{H}, D_{H} and J_{H} gene segments and the Ig V_{κ} and J_{κ} gene segments are human.

21. The genetically modified non-human animal of claim 19, wherein the TCRα and TCRβ constant region genes are of endogenous species origin.

22. The genetically modified non-human animal of claim 21, wherein the TCRα and TCRβ constant region genes are of endogenous species origin.

23. The genetically modified non-human animal of claim 22, wherein the genetically modified non-human animal is a rat or a mouse.

24. The genetically modified non-human animal of claim 19, wherein the genetically modified non-human animal does not express a functional TCR.

25. The genetically modified non-human animal of claim 19, wherein the first CAR locus is located at an endogenous TCRβ locus and the second CAR locus is located at an endogenous TCRα locus.

26. The genetically modified non-human animal of claim 19, wherein the genetically modified non-human animal expresses one or more chimeric MHC class I α chain polypeptides comprising a human extracellular domain and a cytoplasmic domain of endogenous species origin.

27. The genetically modified non-human animal of claim 19, wherein the genetically modified non-human animal expresses a human β-2-microglobulin polypeptide.

28. The genetically modified non-human animal of claim 19, wherein the genetically modified non-human animal expresses one or more chimeric MHC class II α chain polypeptides comprising a human extracellular domain and a cytoplasmic domain of endogenous species origin.

29. The genetically modified non-human animal of claim 19, wherein the genetically modified non-human animal expresses one or more chimeric MHC class II β chain polypeptides comprising a human extracellular domain and a cytoplasmic domain of endogenous species origin.

30. The genetically modified non-human animal of claim 19, wherein the genetically modified non-human animal expresses a chimeric CD8 α chain polypeptide comprising a human extracellular immunoglobulin domain and a cytoplasmic domain of endogenous species origin.

31. The genetically modified non-human animal of claim 19, wherein the genetically modified non-human animal expresses a chimeric CD8 β chain polypeptides comprising a human extracellular immunoglobulin domain and a cytoplasmic domain of endogenous species origin.

32. The genetically modified non-human animal of claim 19, wherein the genetically modified non-human animal expresses a chimeric CD4 polypeptide comprising a human D1 immunoglobulin domain, a human D2 immunoglobulin domain, a human D3 immunoglobulin domain, a D4 immunoglobulin domain, and a cytoplasmic domain of endogenous species origin.

33. The genetically modified non-human animal of claim 19, wherein the CAR has binding specificity for a peptide/MHC complex.

34. The genetically modified non-human animal of claim 19, wherein the CAR is expressed on T cells of the genetically modified non-human animal.

35. The genetically modified non-human animal of claim 19, wherein the genetically modified non-human animal is a rodent.

36. The genetically modified non-human animal of claim 35, wherein the rodent is a mouse.

37. A method of making T cell expressing a CAR specific to a peptide presented on a MHC comprising:
(a) exposing a genetically modified non-human animal of claim 20 to an antigen comprising a peptide or a nucleic acid encoding an antigen comprising a peptide such that the peptide is presented on a MHC in the non-human animal; and
(b) obtaining a T cell expressing a CAR specific for the peptide presented on the MHC from the genetically modified non-human animal of (a).

38. The method of claim 37, wherein the peptide is administered as part of a peptide/MHC complex.

39. A method of making T cell hybridoma expressing a CAR specific to a peptide presented on a MHC comprising:
(a) exposing a genetically modified non-human animal of claim 20 to an antigen comprising a peptide or a nucleic acid encoding an antigen comprising a peptide such that the peptide is presented on a MHC in the non-human animal;
(b) obtaining a T cell expressing a CAR specific for the peptide presented on the MHC from the genetically modified non-human animal of (a); and
(c) making a T cell hybridoma from the T cell of step (b).

40. The method of claim 39, wherein the peptide is administered as part of a peptide/MHC complex.

41. A method for making a nucleic acid encoding an Ig variable domain specific to a peptide presented on a MHC comprising:
(a) exposing a non-human animal of claim 20 to an antigen comprising a peptide or a nucleic acid encoding an antigen comprising a peptide such that the peptide is presented on a MHC in the non-human animal;
(b) obtaining a T cell expressing a CAR specific for the peptide presented on the MHC from the genetically modified non-human animal of (a); and
(c) isolating a nucleic acid encoding an Ig variable domain of the CAR from the T cell.

42. The method of claim 41, wherein the peptide is administered as part of a peptide/MHC complex.

43. A method for making an antibody specific to a peptide presented on a MHC comprising:
(a) exposing a non-human animal of claim 20 to an antigen comprising a peptide or a nucleic acid encoding an antigen comprising a peptide such that the peptide is presented on a MHC in the non-human animal;
(b) obtaining a T cell expressing a CAR specific for the peptide presented on the MHC from the genetically modified non-human animal of (a);
(c) isolating a nucleic acid encoding an Ig variable domain of the CAR from the T cell;
(d) operably linking the nucleic acid encoding the Ig variable domain with an Ig constant domain in a host cell; and
(e) culturing the host cell under conditions such that the host cell expresses an antibody comprising the Ig variable domain and the Ig constant domain.

44. The method of claim 43, wherein the peptide is administered as part of a peptide/MHC complex.
